(19) United States
(12) Patent Application Publication
(10) Pub. No.: US 2003/0212024 A1
(43) Pub. Date: Nov. 13, 2003

(54) Compositions and methods for cell dedifferentiation and tissue regeneration

(76) Inventors: Mark T Keating, Newton, MA (US); Shannon J Odelberg, Salt Lake City, UT (US); Kenneth D Poss, Brookline, MA (US)

Correspondence Address: Matthew P Vincent, Ropes & Gray, One International Place, Boston, MA, 02110-2624, US

(21) Appl. No.: 10/275,828

(22) PCT Filed: May 14, 2001
(86) PCT No.: PCT/US01/15582

(51) Int. Cl. A61K 48/00; C12N 5/08; C12N 15/85

(52) U.S. Cl. 514/44; 435/455; 435/366

(57) ABSTRACT

The present invention is directed to methods and compositions to induce cellular dedifferentiation and tissue regeneration in vitro and in vivo.

### RELATED APPLICATIONS

This application claims priority to U.S. provisional application Serial No. 60/204,080 filed May 12, 2000, U.S. provisional application Serial No. 60/204,081 filed May 12, 2000, and U.S. provisional application Serial No. 60/204,082 filed May 12, 2000, which are incorporated herein by reference in their entirety.

### BACKGROUND OF THE INVENTION

The present invention is directed to compositions that promote cellular dedifferentiation and tissue regeneration. It also is directed to methods of inducing cellular dedifferentiation, proliferation, and regeneration.

Morgan (Morgan, 1901) coined the term epimorphosis to refer to the regenerative process in which cellular proliferation precedes the development of a new anatomical structure. An adult urodele, e.g., a newt or axolot1, are known to be capable of regenerating its limbs, tail, upper and lower jaws, retinas, eye lenses, dorsal crest, spinal cord, and heart ventricle (Becker et al., 1974; Brockes, 1997; Davis et al., 1990), while teleost fish, such as Danio rerio, (zebrafish), are known to regenerate their fins and spinal cord (Johnson and Weston, 1995; Zottoli et al., 1994). Echinoderms and crustaceans are likewise capable of regeneration. With the exception of liver, mammals, such as humans, lack this remarkable regenerative capability.

Mammals typically heal an injury, whether induced from trauma or degenerative disease, by replacing the missing tissue with scar tissue. Wound healing, which is distinct from tissue regeneration, results in scar tissue that has none of the specific functions of the cell types that it replaced, except the qualities of tissue integrity and strength. For example, cardiac injuries, such as from a heart attack, result in cardiac muscle that dies. Instead of new cardiac muscle replacing the dead cells, scar tissue forms. The burden of contraction, once shouldered by the now missing cells, is passed on to surrounding areas, thus increasing the workload of existing cells. For optimal cardiac performance, the dead tissue would need to be replaced with cardiac cells (regeneration).

The molecular and cellular mechanisms that govern epimorphic regeneration are incompletely defined. The first step in this process is the formation of a wound epithelium, which occurs within the first 24 hours following amputation. The second step involves the dedifferentiation of cells proximal to the amputation plane. These cells proliferate to form a mass of pluripotent cells, known as the regeneration blastema, which will eventually redifferentiate to form the lost structure. Although cellular dedifferentiation has been demonstrated in newts, terminally-differentiated mammalian cells are thought to be incapable of reversing the differentiation process (Andres and Walsh, 1996; Walsh and Perlman, 1997). Several mechanisms could explain the lack of cellular plasticity in mammalian cells: (1) the extracellular factors that initiate dedifferentiation are not adequately expressed following amputation; (2) the intrinsic cellular signaling pathways for dedifferentiation are absent; (3) differentiation factors are irreversibly expressed in mammalian cells; and (4) structural characteristics of mammalian cells make dedifferentiation impossible.

Though differentiated, newt myotubes are not locked into a G₀/G₁ state (Hay and Fischman, 1961; Tanaka et al., 1997) and thus are capable of dedifferentiation. In contrast, mammalian skeletal muscle cells are thought to be terminally-differentiated (Andres and Walsh, 1996; Walsh and Perlman, 1997). Normal (non-transformed, non-oncogenic) mammalian myotubes have not been observed to reenter the cell cycle or dedifferentiate in vitro or in vivo. In contrast, oncogenic mammalian cells have been observed to re-enter the cell cycle and proliferate (Endo and Nadal-Ginard, 1989; Endo and Nadal-Ginard, 1998; Iujvidin et al., 1990; Novitch et al., 1996; Schneider et al., 1994; Tiainen et al., 1996). However, these cells are abnormal and cannot participate in regeneration. The ability to dedifferentitate non-oncogenic mammalian cells is a long-sought goal, which the current invention achieves.

While artificial organs, organ transplants, prostheses and other means to substitute for missing tissues, organs, and appendages have improved the quality of life of many who suffer from these problems, all of these methods are fraught with complications and high costs. For example, those lucky enough to receive tissue and organ transplants must be administered expensive anti-rejection drugs for the life of the transplant. In addition to their expense, prostheses suffer from an inability to replace the fill function of the missing appendage.

In addition, current bio-mediated tissue and organ replacement techniques also suffer from significant disadvantages. Tissue engineering, the approach of replacing tissue by culturing in vitro cells onto a biomaterial substrate and then transplanting to anindividual (a mammalian, preferably a human, subject), is hampered by cost, time, and the result is a structure that does not have all of the intrinsic functions and morphology of the tissue it replaces. Likewise, an approach that exploits stem cells ex vivo is similarly hampered by time, where stem cells must be purified from bone marrow or aborted fetuses (also representing limited sources and regulatory resistance), manipulated in vitro, and then the cells introduced into an individual at the site of injury.

The current invention circumvents ex vivo and in vitro approaches, as well as allowing for regeneration of tissue that resembles that of thehost. Regeneration occurs at the site of injury by dedifferentiating the cells in vivo, creating stem cells, and then allowing the stem cells to redifferentiate or newly differentiate into the cells and structures of the host tissue or organ. Such an approach has a broad range of application.

### BRIEF SUMMARY OF THE INVENTION

The invention provides compositions and methods for dedifferentiating cells in vivo and in vitro. The invention also provides compositions and methods for the regeneration of cells, tissue and organs in vivo and in vitro. The present inventors have now discovered that an extract from newt, as well as purified components therefrom, can be used to achieve this and other objectives as discussed herein.

### DETAILED DESCRIPTION OF THE INVENTION

The present invention provides methods and compositions for dedifferentiating cells. Although previously thought to be committed to their differentiated fate, differentiated cells can be dedifferentiated. In certain embodiments, the compositions of the invention include, but are not limited to, polypeptides, nucleic acids, or combinations of these. Dedifferentiation can be accomplished in vitro, in vivo, and ex vivo.

Regeneration extracts (RE; referring to an extract from any animal that regenerates, preferably newt, most preferably, RNLE, hRNLE, and RNLE-purified components), growth factors (GFs), and msx1 are collectively referred to as Regeneration/Dedifferentiation Factors, or RDF.

I. Embodiments

The following embodiments are given as examples of various ways to practice the invention. Many different versions will be immediately apparent to one of skill in the various arts to which this invention pertains.

A. In Vivo

The compositions of the invention can be used in vivo to dedifferentiate cells. Dedifferentiation of cells at the site of an injury, whether trauma or disease-induced, is an early step in the regeneration of cells, tissue and organs. Cells that have been dedifferentiated by the methods and compositions of the invention have regressed in a developmental pathway, such that they resemble stem cells and have become pluripotent, or even totipotent.

Regenerating newt limb extract (RNLE), its humanized form (hRNLE), or purified factors, is applied at the site of injury at the time of, or soon after injury. In some cases, these compositions may be applied to an injury after healing with scar tissue; in such cases, it may be desirable to re-injure the tissue to re-initiate cellular dedifferentiation. In some cases, various components of the compositions may be applied in sequence to enhance dedifferentiation. RE may be delivered to the site of injury in any manner known in the pharmaceutical arts; application may be continuous, instant, or re-applied over a time course during dedifferentiation. RE may be used to regenerate damaged cells, tissue or organs.

Growth factors (GFs) may also be applied to a site of injury to induce cellular dedifferentiation. Sometimes, only one growth factor may be applied; or it might be more advantageous to apply several at once. GFs may be applied at the site of injury at the time of injury; subsequent to the injury, but before scar tissue formation commences; or, after the injury has healed, in which case the damaged tissue or scar may be removed, incurring an injury de novo, and then applying growth factors. GFs may be delivered in any manner known in the pharmaceutical arts; application may be continuous, instant, or re-applied over a time course during dedifferentiation. Injury may be caused by disease or trauma. GFs from the family of fibroblast growth factors (Fgfs) are preferred in some cases. GFs may be used to regenerate damaged cells, tissue or organs.

Intracellular components may also be applied in vivo at the site of injury to dedifferentiate cells, such as the gene msx1, its polypeptide product, or msx1 polypeptide fused such that cellular uptake is induced. Msx1 may be applied at the time of injury, subsequent to the injury, but before scar tissue formation, or at the site of a healed injury, in which case the tissue may be re-injured before msx1 application. Msx1 may be applied in any manner known in the pharmaceutical arts; application may be continuous, instant, or re-applied over a time course during dedifferentiation. The injury may be due to disease or to trauma. Msx1 may be used to regenerate damaged cells, tissue or organs.

In some instances, a combination of RE, GFs, and msx1 may be preferred In other cases, a sequence of the various components may be advantageous; for example, the application of RE may be first desired, followed by GF application and/or msx1.

B. Ex Vivo/In Vitro

To repair an injury induced by disease or trauma, the compositions and methods of the invention may be applied to a procedure wherein differentiated cells are removed from the injured subject, dedifferentiated in culture, and then either re-introduced into the affected individual at the site of injury or, while still in culture, the dedifferentiated cells are manipulated to follow specific differentiation pathways before reintroduction into theindividual. Differentiation pathways include, but are not limited to, adipocytes, chondrocytes, osteogenic cells, and muscle cells.

Cells may be removed from a subject by any method known in the medical arts that is appropriate to the location of the desired cells. Cells are then cultured in vitro, where they may be dedifferentiated using any of the methods and compositions of the inventions, including applying components of RDF. Any cell culture methods known in the arts may be used, or if unknown, one of skill in the art may easily determine the appropriate culture conditions. If desired, the cells may be expanded before reintroducing back to a site of injury in the affected individual. The injury may be recent, in the process of forming scar tissue, or healed. In the latter two cases, the site of injury may be re-injured to create a favorable environment for regeneration. The cells may be delivered to the site of injury by any method known in the medical arts and that is appropriate to the location of the injury and to the cells being delivered.

C. Specific Embodiments

1. Dedifferentiation of Cells using Regenerating Newt Limb Extract

During the dedifferentiation stage of newt limb regeneration, cleaved muscle cell products near the amputation plane contribute significantly to the formation of the blastema. The dedifferentiated muscle cells reenter the cell cycle and actively synthesize protein all within the first week after amputation. Myoblasts are mononucleated skeletal myocytes that proliferate when cultured in the presence of growth factors. These cells are committed to the myogenic lineage through expression of the muscle regulatory factors myoD and/or myf-5. When grown to confluency and deprived of growth factors, these myocytes enter the terminal differentiation pathway and begin to express, in succession, a number of muscle differentiation factors. These include myogenin, the cdk inhibitor p21/WAF1, activated retinoblastoma protein, and the muscle contractile proteins (e.g., myosin heavy chain and troponin T). The differentiating cells align along their axes and fuse to form terminally-differentiated myotubes capable of muscle contraction.

A protein extract, RNLE, from early regenerating limb tissue (days 0-5) in newts induced the dedifferentiation of both newt and murine myotubes in culture. Thus, mammalian (murine) myotubes are capable of dedifferentiating in response to dedifferentiation signals received from regenerating newt limbs. Thus, the present invention provides a composition for dedifferentiating mammalian tissue comprising one or more proteins extracted from newt tissue. RNLE extract can therefore be used to dedifferentiate tissue from, for example, humans. RNLE extract may be applied in vivo or to cells in vitro.

2. Use of msx1 to Dedifferentiate Cells

Msx1 is a homeobox-containing transcriptional repressor. Msx1 is expressed in the early regeneration blastema (Simon et al., 1995), and its expression in the developing mouse limb demarcates the boundary between the undifferentiated (msx1 expressing) and differentiating (no msx1 expression) cells (Hill et al., 1989; Robert et al., 1989; Simon et al., 1995). Furthermore, ectopic expression of either murine or human msx1 will inhibit in vitro myogenesis in cultured mouse cells (Song et al., 1992; Woloshin et al., 1995).

A method to dedifferentiate cells by expression of msx1 is presented. The nucleotide sequence of mouse msx1 is presented in Table 1 (SEQ ID NO: 1); the polypeptide encoded by SEQ ID NO: 1 is presented in Table 2 (SEQ ID NO: 2). The present invention demonstrates that the combined effects of growth medium and ectopic msx1 expression can cause mouse C2C12 myotubes to dedifferentiate to a pool of proliferating, pluripotent stem cells that are capable of redifferentiating into several cell types, including chondrocytes, adipocytes, osteogenic cells, and myotubes. Thus, terminally-differentiated mammalian cells, like their urodele counterparts, are capable of dedifferentiating to pluripotent stem cells when challenged with the appropriate signals, as provided herein. Msx1 and msx1 analogs can be applied, for example, to human cells, in vivo and in vitro to induce cellular dedifferentiation.

TABLE 1
Mus musculus homeo box, msh-like 1 (Msx1), mRNA (SEQ ID NO:1);
Accession NM_010835

| | |
|---|---|
| ggaacccagg agctcgcaga agccggtcag gagctcgcag aagccggtcg cgctcccagc | 60 |
| ctgcccgaaa cccatgatcc agggctgtct cgagctgcgg ctggaggggg ggtccggctc | 120 |
| tgcatggccc cggctgctgc tatgacttct ttgccactcg gtgtcaaagt ggaggactcc | 180 |
| gccttcgcca agcctgctgg gggaggcgtt ggccaagccc ccggggctgc tgcggccacc | 240 |
| gcaaccgcca tgggcacaga tgaggagggg gccaagccca aagtgcccgc ttcactcctg | 300 |
| cccttcagcg tggaggccct catggccgat cacaggaagc ccggggccaa ggagagcgtc | 360 |
| ctggtggcct ccgaaggggc tcaggcagcg ggtggctcgg tgcagcactt gggcacccgg | 420 |
| cccgggtctc tgggcgcccc ggatgcgccc tcctcgccgc ggcctctcgg ccatttctca | 480 |
| gtcggaggac tcctcaagct gccagaagat gctctggtga aggccgaaag ccccgagaaa | 540 |
| ctagatcgga ccccgtggat gcagagtccc cgcttctccc cgcccccagc cagacggctg | 600 |
| accacagctc agctgctggc tctggagcgc aagttccgcc agaagcagta cctgtctatt | 720 |
| gccgagcgcg cggaattctc cagctcgctc agcctcaccg agacccaggt gaagatctgg | 780 |
| ttccagaacc gtcgcgctaa ggccaagaga ctgcaggagg cggagctgga gaagctgaag | 840 |
| atggccgcga aacccatgtt gccgcctgct gccttcggcc tctcttttcc tcttggcggt | 900 |
| cctgcagctg cgggcgcctc actctacagt gcctctggcc ctttccagcg cgccgcgctg | 960 |
| cctgtagcgc ccgtgggact ctacaccgcc catgtaggct acagcatgta ccacctgact | 1020 |
| taggtgggtc cagagtcacc tccctgtggt gccatcccct ccccagccac ctctttgagc | 1080 |
| agagcagcgg gagtccttcc taggaagctc tgctgcccta taccacctgg tcccttctct | 1140 |
| taaacccctt gctacacact tcctcctggt tgtcgcttcc taaaccttcc tcatctgacc | 1200 |
| ccttctggga agaaaaagaa ttggtcggaa gatgttcagg tttttcgagt tttttctaga | 1260 |
| tttacatgcg caagttataa aatgtggaaa ctaaggatgc agaggccaag agatttatcc | 1320 |
| gtggtcccca gcagaattag aggctgaagg agaccagagg ccaaaaggac tagaggccat | 1380 |
| gagactccat cagctgcttc cggtcctgaa accaggcagg acttgcacag agaaattgct | 1440 |
| aagctaatcg gtgctccaag agatgagccc agccctatag aaagcaagag cccagctcct | 1500 |
| tccactgtca aactctaagc gctttggcag caaagcattg ctctgagggg gcagggcgca | 1560 |
| tgctgctgct tcaccaaggt aggttaaaga gactttccca ggaccagaaa aaaagaagta | 1620 |
| aaaaaaaaaa aaaaaaaaaa aaaaaaaaaa caaatctgtt ctattaacag tacattttcg | 1680 |
| tggctctcaa gcatcccttt tgaagggact ggtgtgtact atgtaatata ctgtatattt | 1740 |
| gaaattttat tatcatttat attatagcta tatttgttaa ataaattaat tttaagctac | 1800 |
| an 1802 | |

TABLE 2
Mus musculus homeo box, msh-like 1 (Msx1), polypeptide
(SEQ ID NO:2); Accession NM_010835

```
Met Ala Pro Ala Ala Ala Met ThrSer Leu Pro Leu Gly Val Lys Val
1 5 10 15
Glu Asp Ser Ala Phe Ala Lys Pro Ala Gly Gly Gly Val Gly Gln Ala
                        20 25 30
Pro Gly Ala Ala Ala Ala Thr Ala Thr Ala Met Gly Thr Asp Glu Glu
                35 40 45
Gly Ala Lys Pro Lys Val Pro Ala Ser Leu Leu Pro Phe Ser Val Glu
        50 55 60
Ala Leu Met Ala Asp His Arg Lys Pro Gly Ala Lys Glu Ser Val Leu
65 70 75 80
Val Ala Ser Glu Gly Ala Gln Ala Ala Gly Gly Ser Val Gln His Leu
                                85 90 95
Gly Thr Arg Pro Gly Ser Leu Gly Ala Pro Asp Ala Pro Ser Ser Pro
                        100 105 110
Arg Pro Leu Gly His Phe Ser Val Gly Gly Leu Leu Lys Leu Pro Glu
                115 120 125
Asp Ala Leu Val Lys Ala Glu Ser Pro Glu Lys Leu Asp Arg Thr Pro
        130 135 140
Trp Met Gln Ser Pro Arg Phe Ser Pro Pro Pro Ala Arg ARg Leu Ser
145 150 155 160
Pro Pro Ala Cys Thr Leu Arg Lys His Lys Thr Asn Arg Lys Pro Arg
                                165 170 175
Thr Pro Phe Thr Thr Ala Gln Leu Leu Ala Leu Glu Arg Lys Phe Arg
                        180 185 190
Gln Lys Gln Thr Leu Ser Ile Ala Glu Arg Ala Glu Phe Ser Ser Ser
                195 200 205
Leu Ser Leu Thr Glu Thr Gln Val Lys Ile Trp Phe Gln Asn Arg Arg
        210 215 220
Ala Lys Ala Lys Arg Leu Gln Glu Ala Glu Leu Glu Lys Leu Lys Met
225 230 235 240
Ala Ala Lys Pro Met Leu Pro Pro Ala Ala Phe Gly Leu Ser Phe Pro
                                245 250 255
Leu Gly Gly Pro Ala Ala Ala Gly Ala Ser Leu Tyr Ser Ala Ser Gly
                        260 265 270
Pro Phe Gln Arg Ala Ala Leu Pro Val Ala Pro Val Gly Leu Tyr Thr
                275 280 285
Ala His Val Gly Tyr Ser Met Tyr His Leu Thr
        290 295
```

3. Use of Fibroblast Growth Factors to Promote Tissue Regeneration

The inventors demonstrate herein that Fgf signaling can mediate regeneration. Fgf, which binds Fgf receptor (Fgfr), is involved in mammalian wound healing and tumor angiogenesis and has numerous roles in embryonic development, including induction and/or patterning during organogenesis of the limb, tooth, brain, and heart.

Members of the Fgf signaling pathway are expressed in the epidermis as well as mesenchymal tissue during blastema formation and outgrowth stages. The inventors tested the function of Fgf signaling during Zebrafish fin regeneration, using a specific pharmacologic inhibitor of Fgfr1. Use of this agent revealed distinct requirements for Fgf signaling in induction and maintenance of blastemal cells, and suggested an additional role in patterning the regenerate. Thus, Fgf and like factors, may be used to dedifferentiate cells and to regenerate tissue in mammal, including humans.

4. Stem Cell Production In Vitro

In one embodiment, the invention provides methods to establish stem cells in vitro. Such stem cells are dedifferentiated from cells provided, for example, from an individual or a tissue culture cell line. Dedifferentiation may be achieved by applying components of RDF. These stem cells can then be directed down different differentiation pathways by in vitro manipulation, or by transplanting back into the individual.

In another embodiment, the invention provides methods to establish pluripotent cells in vitro. Such pluripotent cells are derived from cells provided, for example, from a subject or a tissue culture cell line. Pluripotency may be achieved by applying RDF components to cause cells to dedifferentiate and take on pluripotent characteristics. Such cells can then be directed down different differentiation pathways by in vitro manipulation and then implanted into a subject, or by directly implanting into a subject.

In another embodiment, the invention provides methods to dedifferentiate muscle-derived cells, such that these cells resemble stem or pluripotent cells. In another embodiment, these cells can be driven down other differentiation pathways, such as adipocytes, chondrocytes, myotubes or osteoblasts.

5. Using RDF

Using RE will regenerate injured cells, tissue or organs. At the site of injury, RE may be applied, recapitulating the steps in regeneration seen in newts. Similarly, msx1 and/or Fgf can be used to dedifferentiate cells at the site of injury to promote cell, tissue or organ regeneration. For example, the injured tissue may be in a mammal; the mammal may be a human, and the injured site may be the consequence is of trauma or disease.

Degenerative diseases and other medical conditions that might benefit from regeneration therapies include, but are not limited to: atherosclerosis, coronary artery disease, obstuctive vascular disease, myocardial infarction, dilated cardiomyopathy, heart failure, myocardial necrosis, valvular heart disease, mitral valve prolapse, mitral valve regurgitation, mitral valve stenosis, aortic valve stenosis, and aortic valve regurgitation, carotid artery stenosis, femoral artery stenosis, stroke, claudication, and aneurysm; cancer-related conditions, such as structural defects resulting from cancer or cancer treatments; the cancers such as, but not limited to, breast, ovarian, lung, colon, prostate, skin, brain, and genitourinary cancers; skin disorders such as psoriasis; joint diseases such as degenerative joint disease, rheumatoid arthritis, arthritis, osteoarthritis, osteoporosis and ankylosing spondylitis; eye-related degeneration, such as cataracts, retinal and macular degenerations such as maturity onset, macular degeneration, retinitis pigmentosa, and Stargardt's disease; aural-related degeneration, such as hearing loss; lung-related disorders, such as chronic obstructive pulmonary disease, cystic fibrosis, interstitial lung disease, emphysema; metabolic disorders, such as diabetes; genitourinary problems, such as renal failure and glomerulonephropathy; neurologic disorders, such as dementia, Alzheimer's disease, vascular dementia and stroke; and endocrine disorders, such as hypothyroidism. Finally, regeneration therapies from the methods and compositions of the invention may be very useful and beneficial for traumas to skin, bone, joints, eyes, neck, spinal column, and brain, for example, that result in injuries that would normally result in scar formation.

In addition to limb regeneration seen in the newt, like the newt, it is contemplated that other structures in mammals may be regenerated, such as skin, bone, joints, eyes (epithelium, retina, lens), lungs, heart, blood vessels and other vasculature, kidneys, pancreas, reproductive organs and nervous tissue (stroke, spinal cord injuries).

II. Definitions

Unless defined otherwise, all technical and scientific terms have the same meaning as is commonly understood by one of skill in the art to which this invention belongs.

The recommendations of (Demerec et al., 1966) where these are relevant to nomenclature are adapted herein. To distinguish between genes (and related nucleic acids) and the proteins that they encode, the abbreviations for genes are indicated by italicized (or underlined) text while abbreviations for the proteins are not italicized. Thus, msx1 or msx1 refers to the homeobox msh1-like (msx1) nucleotide sequence that encodes homeobox msh1-like (msx1) polypeptide.

“Isolated,” with respect to a molecule, means a molecule that has been identified and separated and/or recovered from a component of its natural environment. Contaminant components of its natural environment are materials that interfere with diagnostic or therapeutic use.

“Epimorphosis” refers to the process in which cellular proliferation precedes the development of a new anatomical structure; reproduction or reconstitution of a lost or injured part (neogenesis). While regeneration may recapitulate embryonic development, it may also involve metaplasia, the transformation of one differentiated cell type into another.

A cell that is “totipotent” is one that may differentiate into any type of cell and thus form a new organism or regenerate any part of an organism.

A “pluripotent” cell is one that has an unfixed developmental path, and consequently may differentiate into various specialized types of tissue elements, for example, such as adipocytes, chondrocytes, muscle cells, or osteoclasts. Pluripotent cells resemble totipotent cells in that they are able to develop into other cell types, however, various pluripotent cells may be limited in the number of developmental pathways they may travel.

A “marker” is used to determine the differentiated state of a cell. Markers are characteristics, whether morphological or biochemical (enzymatic), particular to a cell type, or molecules expressed by the cell type. Preferably, such markers are proteins, and more preferably, possesses an epitope for antibodies or other binding molecules available in the art. However, a marker may consist of any molecule found in a cell, including, but not limited to, proteins (peptides and polypeptides), lipids, polysaccharides, nucleic acids and steroids.

Markers may be detected by any method available to one of skill in the art. In addition to antibodies (and all antibody derivatives) that recognize and bind at least one epitope on a marker molecule, markers may be detected using analytical techniques, such as by protein dot blots, sodium dodecyl sulfate polyacrylamide gel electrophoresis (SDS-PAGE), or any other gel system that separates proteins, with subsequent visualization of the marker (such as Western blots), gel filtration, affinity column purification; morphologically, such as fluorescent-activated cell sorting (FACS), staining with dyes that have a specific reaction with a marker molecule (such as ruthenium red and extracellular matrix molecules), specific morphological characteristics (such as the presence of microvilli in epithelia, or the pseudopodia/filopodia in migrating cells, such as fibroblasts and mesenchyme); and biochemically, such as assaying for a enzymatic product or intermediate, or the overall composition of a cell, such as the ratio of protein to lipid, or lipid to sugar, or even the ratio of two specific lipids to each other, or polysaccharides. In the case of nucleic acid markers, any known method may be used. If such a marker is a nucleic, PCR, RT-PCR, in situ hybridization, dot-blot hybridization, Northern blots, Southern blots and the like may be used, coupled with suitable detection methods.

In any case, a marker, or more usually, the combination of markers, will show specificity to a cell type. Myofibrils, for example, are characteristic of solely muscle cells; axons are relegated to nervous tissue, cadherins are typical of epithelia, β2-integrins to white blood cells of the immune system, and a high lipid content characteristic of oligodendrocytes while lipid droplets are unique to adipocytes. The preceding list is meant to serve as a nonlimiting example.

“Differentiation” describes the acquisition or possession of one or more characteristics or functions different from that of the original cell type. A differentiated cell is one that has a different character or function from the surrounding structures or from the precursor of that cell (even the same cell). Differentiation gives rise from a limited set of cells (for example, in vertebrates, the three germ layers of the embryo: ectoderm, mesoderm and endoderm) to cellular diversity, creating all of the many specialized cell types that comprise an individual.

Differentiation is a developmental process whereby cells assume a specialized phenotype, i.e., acquire one or more characteristics or functions distinct from other cell types. In most uses, the differentiated phenotype refers to a cell phenotype that is at the mature endpoint in some developmental pathway. In many but not all tissues, the process of differentiation is coupled with exit from the cell cycle—in these cases, the cells lose or greatly restrict their capacity to proliferate.

“Dedifferentiation” describes the process of a cell “going back” in developmental time to resemble that of its progenitor cell. An example of dedifferentiation is the temporal loss of epithelial cell characteristics during wounding and healing. Dedifferentiation may occur in degrees: in the afore-mentioned example of wound healing, dedifferentiation progresses only slightly before the cells re-differentiate to recognizable epithelia. A cell that has greatly dedifferentiated, for example, is one that resembles a stem cell that can give rise to a differentiated cell.

“Muscle cells” are characterized by their principal role: contraction. Muscle cells are usually elongate and arranged in vivo in parallel arrays. The principal components of muscle cells, related to contraction, are the myofilaments. Two types of myofilaments can be distinguished: (1) those composed primarily of actin, and (2) those composed primarily of myosin. While actin and myosin can be found in many other cell types, enabling such cells, or portions, to be mobile, muscle cells have an enormous number of co-aligned contractile filaments that are used to perform mechanical work.

Muscle tissue can be classified into two major classes based on the appearance and location of the contractile cells: (1) striated muscle, containing cross striations, and (2) smooth muscle, which does not contain any cross striations. Striated muscle can be farther subdivided into skeletal muscle and cardiac muscle.

“Skeletal muscle” tissue, in vivo, consists of parallel striated muscle cells, enveloped by connective tissue. Striated muscles cells are also called fibers. Skeletal muscle cells are usually long, multinucleated, and display cross striations. Occasionally satellite cells, much smaller than the skeletal muscle cells, are associated with the fibers.

“Cardiac muscle” consists of long fibers that, like skeletal muscle, are cross-striated. In addition to the striations, cardiac muscle also contains special cross bands, the intercalated discs, which are absent in skeletal muscle. Also unlike skeletal muscle in which the muscle fiber is a single multinucleated protoplasmic unit, in cardiac muscle the fiber consists of mononucleated (sometimes binucleated) cells aligned end-to-end. Cardiac cells often anastomose and conatin many large mitochondria. Usually, injured cardiac muscle is replaced with fibrous connective tissue, not cardiac muscle.

“Smooth muscle” consists of fusiform cells, 20 to 200 μM long, and in vivo, are thickest at the midregion, and taper at each end. While smooth muscle cells have microfilaments, they are not arranged in the ordered, paracrystalline manner of striated muscle. These cells contain numerous pinocytotic vesicles, and with the sacroplasmic reticulum, sequester calcium. Smooth muscle cells will contact each other via gap junctions (or nexus). While some smooth muscle cells can divide, such as those found in the uterus, regenerative capacity is limited, and damaged areas are usually repaired by scar formation.

Other “contractile cells” include myofibroblasts, myoepithelial cells, testicle myoid cells, perineurial cells; although these are not usually anatomically classified as muscle cells.

A “stem cell” describes any precursor cell, whose daughter cells may differentiate into other cell types. In general, a stem cell is a cell capable of extensive proliferation, generating more stem cells (self-renewal) as well as more differentiated progeny. Thus, a single stem cell can generate a clone containing millions of differentiated cells as well as a few stem cells. Stem cells thereby enable the continued proliferation of tissue precursors over a long period of time. Mammalian hematopoictic stem cells migrate to the bone marrow, where they will remain for the duration of the animal's life. Similarly, there are stem cells for such continually renewed tissues as epidermis and sperm. Some stem cells, such as that for skeletal muscle, probably exist during fetal development (Gilbert, 1991).

Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise only to differentiated progeny. Formally, it is possible that cells that begin as stem cells might proceed toward a differentiated phenotype, but then “reverse” and re-express the stem cell phenotype.

Teratocarcinomas also contain stem cells, called embryonal carcinoma cells. Capable of division, they can differentiate into a wide variety of tissues, including gut and respiratory epithelia, muscle, nerve, cartilage, and bone (Gilbert, 1991).

Like stem cells, cells that begin as “progenitor cells” may proceed toward a differentiated phenotype, but then “reverse” and re-express the progenitor cell phenotype. Progenitor cells have a cellular phenotype that is more primitive than a differentiated cell; these cells are at an earlier step along a developmental pathway or progression than fully differentiated cells. Often, progenitor cells also have significant or very high proliferative potential. Progenitor cells may give rise to multiple distinct differentiated cell types or to a single differentiated cell type, depending on the developmental pathway and on the environment in which the cells develop and differentiate.

“Proliferation” refers to an increase in the number of cells in a population (growth) by means of cell division. Cell proliferation results from the coordinated activation of multiple signal transduction pathways, often in response to growth factors and other mitogens. Cell proliferation may also be promoted when cells are released from the actions of intra- or extracellular signals and mechanisms that block or down-regulate cell proliferation.

“Control sequences” are DNA sequences that enable the expression of an operably-linked coding sequence in a particular host organism. Prokaryotic control sequences include promoters, operator sequences, and ribosome binding sites. Eukaryotic cells utilize promoters, polyadenylation signals, and enhancers.

Nucleic acid is “operably-linked” when it is placed into a functional relationship with another nucleic acid sequence. For example, a promoter or enhancer is operably-linked to a coding sequence if it affects the transcription of the sequence, or a ribosome-binding site is operably-linked to a coding sequence if positioned to facilitate translation. Generally, “operably-linked” means that the DNA sequences being linked are contiguous, and, in the case of a secretory leader, contiguous and in reading phase. However, enhancers do not have to be contiguous. Linking is accomplished by conventional recombinant DNA methods.

An “isolated nucleic acid” molecule is purified from the setting in which it is found in nature and is separated from at least one contaminant nucleic acid molecule. Isolated msx1 molecules are distinguished from the specific msx1 molecule, as it exists in cells. However, an isolated msx1 molecule includes msx1 molecules contained in cells that ordinarily express msx1 where, for example, the nucleic acid molecule is in a chromosomal location different from that of natural cells.

When the molecule is a “purified polypeptide,” the polypeptide will be purified (1) to obtain at least 15 residues of N-terminal or internal amino acid sequence using a sequenator, or (2) to homogeneity by SDS-PAGE under non-reducing or reducing conditions using Coomassie blue or silver stain. Isolated polypeptides include those expressed heterologously in genetically-engineered cells or expressed in vitro, since at least one component of msx1 natural environment will not be present. Ordinarily, isolated polypeptides are prepared by at least one purification step.

A polypeptide or polypeptide fragment retains a biological and/or an immunological activity of the native or naturally-occurring polypeptide. Immunological activity refers to the ability to induce the production of an antibody against an antigenic epitope possessed by a native polypeptide; biological activity refers to a function, either inhibitory or stimulatory, caused by a native msx1 that excludes immunological activity. A biological activity of msx1 includes, for example, modulating cellular dedifferentiation.

“Derivatives” of nucleic acid sequences or amino acid sequences are formed from the native compounds either directly or by modification or partial substitution. “Analogs” are nucleic acid sequences or amino acid sequences that have a structure similar to, but not identical to, the native compound but differ from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions (Ausubel et al., 1987).

A “homologous nucleic acid sequence” or “homologous amino acid sequence,” or variations thereof, refer to sequences characterized by homology at the nucleotide level or amino acid level as discussed above. Homologous nucleotide sequences encode those sequences coding for isoforms of msx1. Isoforms can be expressed in different tissues of the same organism as a result of, for example, alternative splicing of RNA Alternatively, different genes can encode isoforms. Homologous nucleotide sequences include nucleotide sequences encoding for msx1 of other species, including, but not limited to: vertebrates, and thus can include, e.g., human, frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding hummsx1. Homologous nucleic acid sequences include those nucleic acid sequences that encode conservative amino acid substitutions (see below) in SEQ ID NO: 2, as well as a polypeptide possessing msx1 biological activity. Various biological activities of the msx1 are described below.

An “open reading frame” (ORF) is a nucleotide sequence that has a start codon (ATG) and terminates with one of the three “stop” codons (TAA, TAG, or TGA). In this invention, however, an ORF may be any part of a coding sequence that may or may not comprise a start codon and a stop codon. For example, the ORF of msx1 gene encodes msx1; preferable msx1 ORFs encode at least 50 amino acids of msx1.

In general, a “growth factor” is a substance that promotes cell growth and development by directing cell maturation and differentiation. Growth factors also mediate tissue maintenance and repair. Growth factors are ligated by specific receptors and act at very low concentrations.

“Fibroblast growth factors” (Fgfs) belong to a class of growth factors consisting of a large family of short polypeptides that are released extracellularly and bind with heparin to dimerize and activate specific receptor tyrosine kinases (Fgfrs). Fgf signaling is involved in mammalian wound healing and tumor angiogenesis (Ortega et al., 1998; Zetter, 1998) and has numerous roles in embryonic development, including induction and/or patterning during organogenesis of the limb, tooth, brain, and heart (Crossley et al., 1996; Martin, 1998; Ohuchi et al., 1997; Peters and Balling, 1999; Reifers et al., 1998; Vogel et al., 1996; Zhu et al., 1996).

Fgfs can easily be detected using either functional assays (Baird and Klagsbrun, 1991; Moody, 1993) or antibodies (Research Diagnostics; Flanders, N.J. or Promega, Wis.).

A “mature” form of a polypeptide or protein is the product of a naturally occurring polypeptide or precursor form or proprotein. For example, msx1 can encode a mature msx1. The naturally occurring polypeptide, precursor or proprotein includes, for example, the full-length gene product, encoded by the corresponding gene. Alternatively, it may be defined as the polypeptide, precursor or proprotein encoded by an open reading frame described herein. The product “mature” form arises as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an (open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

An “active” polypeptide or polypeptide fragment retains a biological and/or an immunological activity similar, but not necessarily identical, to an activity of a naturally-occuring (wild-type) polypeptide of the invention, including mature forms. Biological assays, with or without dose dependency, can be used to determine activity. A nucleic acid fragment encoding a biologically-active portion of a polypeptide can be prepared by isolating a portion of a nucleic acid sequence that encodes a polypeptide having biological activity, expressing the encoded portion of the polypeptide and assessing the activity of the encoded portion of msx1. immunological activity refers to the ability to induce the production of an antibody against an antigenic epitope possessed by a native msx1; biological activity refers to a function, either inhibitory or stimulatory, caused by a native msx1 that excludes immunological activity.

Regarding msx1, the invention further encompasses the use of nucleic acid molecules that differ from the nucleotide sequence shown in SEQ ID NO: 1 due to degeneracy of the genetic code and thus encode the same msx1 as that encoded by the nucleotide sequences shown in SEQ ID NO NO: 1. An isolated nucleic acid molecule useful for the invention has a nucleotide sequence encoding a protein having an amino acid sequence shown in SEQ ID NO: 2.

In addition to the msx1 sequence shown in SEQ ID NO: 1, DNA sequence polymorphisms that change the amino acid sequences of msx1 may exist within a population For example, allelic variation among individuals will exhibit genetic polymorphism in msx1. The terms “gene” and “recombinant gene” refer to nucleic acid molecules comprising an open reading frame (ORF) encoding msx1, preferably a vertebrate msx1. Such natural allelic variations can typically result in 1-5% variance in msx1. Any and all such nucleotide variations and resulting amino acid polymorphisms in msx1, which are the result of natural allelic variation and that do not alter the functional activity of msx1 are useful for the methods of the invention Moreover, msx1 from other species that have a nucleotide sequence different than the sequence of SEQ ID NO: 1, are also useful. Nucleic acid molecules corresponding to natural allelic variants and homologues of msx1 cDNAs of the invention can be isolated based on their homology to msx1 of SEQ ID NO: 1 using cDNA-derived probes to hybridize to homologous msx1 sequences under stringent conditions.

“msx1 variant polynucleotide” or “msx1 variant nucleic acid sequence” means a nucleic acid molecule which encodes an active msx1 that (1) has at least about 80% nucleic acid sequence identity with a nucleotide acid sequence encoding a full-length native msx1, (2) a full-length native msx1 lacking the signal peptide, (3) an extracellular domain of msx1, with or without the signal peptide, or (4) any other fragment of a full-length msx1. Ordinarily, msx1 variant polynucleotide will have at least about 80% nucleic acid sequence identity, more preferably at least about 81%, 82%, 83%, 84%, 85%, 86%, 87%, 88%, 89%, 90%, 91%, 92%, 93%, 94%, 95%, 96%, 97%, or 98% nucleic acid sequence identity and yet more preferably at least about 99% nucleic acid sequence identity with the nucleic acid sequence encoding a full-length native msx1. Msx1 variant polynucleotide may encode a full-length native msx1 lacking the signal peptide, an extracellular domain of msx1, with or without the signal sequence, or any other fragment of a full-length msx1. Variants do not encompass the native nucleotide sequence.

Ordinarily, msx1 variant polynucleotides are at least about 30 nucleotides in length, often at least about 60, 90, 120, 150, 180, 210, 240, 270, 300, 450, or 600 nucleotides in length, more often at least about 900 nucleotides in length, or more.

“Percent (%) nucleic acid sequence identity” with respect to msx1-encoding nucleic acid sequences identified herein is defined as the percentage of nucleotides in a candidate sequence that are identical with the nucleotides in the msx1 sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:

% nucleic acid sequence identity=W/Z−100

where W is the number of nucleotides scored as identical matches by the sequence alignment program's or algorithm's alignment of C and D and Z is the total number of nucleotides in D.

When the length of nucleic acid sequence C is not equal to the length of nucleic acid sequence D, the % nucleic acid sequence identity of C to D will not equal the % nucleic acid sequence identity of D to C.

Homologs (i.e., nucleic acids encoding msx1 derived from other species) or other related sequences (e.g., paralogs) can be obtained by low, moderate or high stringency hybridization with all or a portion of the particular sequence of SEQ ID NO: 1 as a probe using methods well known in the art for nucleic acid hybridization and cloning.

The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach is to vary the temperature: higher relative temperatures result in more stringent reaction conditions. (Ausubel et al., 1987) provide an excellent explanation of stringency of hybridization reactions.

To hybridize under “stringent conditions” describes hybridization protocols in which nucleotide sequences at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

(a) High Stringency

“Stringent hybridization conditions” conditions enable a probe, primer or oligonucleotide to hybridize only to its target sequence. Stringent conditions are sequence-dependent and will differ. Stringent conditions comprise: (1) low ionic strength and high temperature washes (e.g., 15 mM sodium chloride, 1.5 mM sodium citrate, 0.1% sodium dodecyl sulfate at 50° C.); (2) a denaturing agent during hybridization (e.g., 50% (v/v) formamide, 0.1% bovine serum albumin, 0.1% Ficoll, 0.1% polyvinylpyrrolidone, 50 mM sodium phosphate buffer (pH 6.5; 750 mM sodium chloride, 75 mM sodium citrate at 42° C.); or (3) 50% formanzide. Washes typically also comprise 5× SSC (0.75 M NaCl, 75 mM sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C. with washes at 42° C. in 0.2× SSC (sodium chloride/sodium citrate) and 50% fonnamide at 55° C., followed by a high-stringency wash consisting of 0.1× SSC containing EDTA at 55° C. Preferably, the conditions are such that sequences at least about 65%, 70%, 75%, 85%, 90%, 95%, 98%, or 99% homologous to each other typically remain hybridized to each other. These conditions are presented as examples and are not meant to be limiting.

(b) Moderate Stringency

“Moderately stringent conditions” use washing solutions and hybridization conditions that are less stringent (Sambrook, 1989), such that a polynucleotide will hybridize to the entire, fragments, derivatives or analogs of SEQ ID NO: 1. One example comprises hybridization in 6× SSC, 5× Denhardt's solution, 0.5% SDS and 100 mg/ml denatured salmon sperm DNA at 55° C., followed by one or more washes in 1× SSC, 0.1% SDS at 37° C. The temperature, ionic strength, etc., can be adjusted to accommodate experimental factors such as probe length. Other moderate stringency conditions are described in (Ausubel et al., 1987; Kriegler, 1990).

(c) Low Stringency

“Low stringent conditions” use washing solutions and hybridization conditions that are less stringent than those for moderate stringency (Sambrook, 1989), such that a polynucleotide will hybridize to the entire, fragments, derivatives or analogs of SEQ ID NO: 1. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency, such as those for cross-species hybridizations are described in (Ausubel et al., 1987; Kriegler, 1990; Shilo and Weinberg, 1981).

In addition to naturally occurring allelic variants of msx1, changes can be introduced by mutation into SEQ ID NO: 1 sequence that incur alterations in the amino acid sequences of the encoded msx1 that do not alter msx1 function. For example, nucleotide substitutions leading to amino acid substitutions at “non-essential” amino acid residues can be made in the sequence of SEQ ID NO: 2. A “non-essential” amino acid residue is a residue that can be altered from the wild-type sequences of the msx1 without altering their biological activity, whereas an “essential” amino acid residue is required for such biological activity. For example, amino acid residues that are conserved among the msx1 are predicted to be particularly non-amenable to alteration. conservative substitutions are well-known in the art.

Useful conservative substitutions are shown in Table A, “Preferred substitutions.” Conservative substitutions whereby an amino acid of one class is replaced with another amino acid of the same type fall within the scope of the subject invention so long as the substitution does not materially alter the biological activity of the compound If such substitutions result in a change in biological activity, then more substantial changes, indicated in Table B as exemplary are introduced and the products screened for msx1 polypeptide's biological activity.

TABLE A
Preferred substitutions

| Original | | Preferred |
|---|---|---|
| residue | Exemplary substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, Ala, | Ile |
| | Phe | |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | Norleucine | |

Non-conservative substitutions that affect (1) the structure of the polypeptide backbone, such as a β-sheet or α-helical conformation, (2) the charge, (3) hydrophobicity, or (4) the bulk of the side chain of the target site can modify msx1 polypeptide's function or immunological identity. Residues are divided into groups based on common side-chain properties as denoted in Table B. Non-conservative substitutions entail exchanging a member of one of these classes for another class. Substitutions may be introduced into conservative substitution sites or more preferably into non-conserved sites.

TABLE B
Amino acid classes

| Class | Amino acids |
|---|---|
| hydrophobic | Norleucine, Met, Ala, Val, Leu, Ile |
| neutral hydrophilic | Cys, Ser, Thr |
| Acidic | Asp, Glu |
| Basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (Carter, 1986; Zoller and Smith, 1987), cassette mutagenesis, restriction selection mutagenesis (Wells et al., 1985) or other known techniques can be performed on the cloned DNA to produce msx1 variant DNA (Ausubel et al., 1987; Sambrook, 1989).

In one embodiment, the isolated nucleic acid molecule comprises a nucleotide sequence encoding a protein, wherein the protein comprises an amino acid sequence at least about 45%, preferably 60%, more preferably 70%, 80%, or 90%, and most preferably about 95% homologous to SEQ ID NO: 1.

One aspect of the invention pertains to the use of, for example, isolated msx1, and biologically active portions, derivatives, fragments, analogs or homologs thereof However, the proceeding section is applicable to all components of RDF; msx1 will be used as an example for illustration purposes. Also provided are polypeptide fragments suitable for use as immunogens to raise anti-msx1 Abs. In one embodiment, a native msx1 can be isolated from cells or tissue sources by an appropriate purification scheme using standard protein purification techniques. In another embodiment, msx1 are produced by recombinant DNA techniques. Alternative to recombinant expression, msx1 can be synthesized chemically using standard peptide synthesis techniques.

(a) msx1 Polypeptides

Msx1 polypeptide includes the amino acid sequence of msx1 whose sequence is provided in SEQ ID NO: 2. The invention also includes a mutant or variant protein any of whose residues may be changed from the corresponding residues shown in SEQ ID NO: 2, while still encoding a protein that maintains msx1 activities and physiological functions, or a functional fragment thereof.

(b) Variant msx1 Polypeptides

In general, msx1 variants that preserve msx1-like function includes any variant in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

“msx1 polypeptide variant” means an active msx1 polypeptide having at least: (1) about 80% amino acid sequence identity with a full-length native sequence msx1 polypeptide sequence, (2) msx1 polypeptide sequence lacking the signal peptide, (3) an extracellular domain of msx1 polypeptide, with or without the signal peptide, or (4) any other fragment of a full-length msx1 polypeptide sequence. For example, msx1 polypeptide variants include msx1 polypeptides wherein one or more amino acid residues are added or deleted at the N- or C-terminus of the full-length native amino acid sequence. Msx1 polypeptide variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, or 98% amino acid sequence identity and most preferably at least about 99% amino acid sequence identity with a full-length native sequence msx1 polypeptide sequence. Msx1 polypeptide variant may have a sequence lacking the signal peptide, an extracellular domain of msx1 polypeptide, with or without the signal peptide, or any other fragment of a full-length msx1 polypeptide sequence. Ordinarily, msx1 variant polypeptides are at least about 10 amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in a disclosed msx1 polypeptide sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

% amino acid sequence identity=X/Y·100

where X is the number of amino acid residues scored as identical matches by the sequence alignment program's or algorithm's alignment of A and B and Y is the total number of amino acid residues in B.

If the length of amino acid sequence A is not equal to the length of amino acid sequence B, the % amino acid sequence identity of A to B will not equal the % amino acid sequence identity of B to A.

(c) Isolated/Purified Polypeptides

An “isolated” or “purified” polypeptide, protein or biologically active fragment is separated and/or recovered from a component of its natural environment. Contaminant components include materials that would typically interfere with diagnostic or therapeutic uses for the polypeptide, and may include enzymes, hormones, and other proteinaceous or non-proteinaceous materials. Preferably, the polypeptide is purified to a sufficient degree to obtain at least 15 residues of N-terminal or internal amino acid sequence. To be substantially isolated, preparations having less than 30% by dry weight of non-msx1 contaminating material (contaminants), more preferably less than 20%, 10% and most preferably less than 5% contaminants. An isolated, recombinantly-produced msx1 or biologically active portion is preferably substantially free of culture medium, i.e., culture medium represents less than 20%, more preferably less than about 10%, and most preferably less than about 5% of the volume of the msx1 preparation Examples of contaminants include cell debris, culture media, and substances used and produced during in vitro synthesis of msx1.

(d) Biologically Active

Biologically active portions of msx1 (or any RE component that is proteinaceous) include peptides comprising amino acid sequences sufficiently homologous to or derived from the amino acid sequences of msx1 (SEQ ID NO: 2) that include fewer amino acids than a full-length msx1, and exhibit at least one activity of msx1. Biologically active portions comprise a domain or motif with at least one activity of a native msx1. A biologically active portion of msx1 can be a polypeptide that is, for example, 10, 25, 50, 100 or more amino acid residues in length Other biologically active portions, in which other regions of the protein are deleted, can be prepared by recombinant techniques and evaluated for one or more of the functional activities of a native msx1.

Biologically active portions of msx1 may have an amino acid sequence shown in SEQ ID NO: 2, or substantially homologous to SEQ ID NO: 2, and retain the functional activity of the protein of SEQ ID NO: 2, yet differ in amino acid sequence due to natural allelic variation or mutagenesis. Other biologically active msx1 may comprise an amino acid sequence at least 45% homologous to the amino acid sequence of SEQ ID NO: 2, and retain the functional activity of native msx1.

(e) Chimeric and Fusion Proteins

Fusion polypeptides are useful in expression studies, cell-localization, bioassays, msx1 purification, and for the purposes of the methods of the invention, for intracellular introduction of msx1 by extracellular application. Msx1 “chimeric protein” or “fusion protein” comprises msx1 fused to a non-msx1 polypeptide. A non-msx1 polypeptide is not substantially homologous to msx1 (SEQ ID NO: 2). Msx1 fusion protein may include any portion of an entire msx1, including any number of the biologically active portions. Msx1 may be fused to the C-terminus of the GST (glutathione S-transferase) sequences. Such fusion proteins facilitate the purification of a recombinant msx1. In certain host cells, (e.g., mammalian), heterologous signal sequence fusions may ameliorate msx1 expression and/or intracellular uptake. For example, residues of the HIV tat protein can be used to encourage intracellular uptake and nuclear delivery (Frankel et al., U.S. Pat. No. 5,804,604, 1998). Additional exemplary fusions are presented in Table C.

Fusion proteins can be easily created using recombinant methods. A nucleic acid encoding msx1 can be fused in-frame with a non-msx1 encoding nucleic acid, to msx1 NH₂— or COO— -terminus, or internally. Fusion genes may also be synthesized by conventional techniques, including automated DNA synthesizers. PCR amplification, using anchor primers that give rise to complementary overhangs between two consecutive gene fragments that can subsequently be annealed and reamplified to generate a chimeric gene sequence (Ausubel et al., 1987), is also useful. Many vectors are commercially available that facilitate sub-cloning msx1 in-frame to a fusion moiety.

TABLE C
Useful fusion polypeptides

| Reporter | in vitro | in vivo | Notes | Reference |
|---|---|---|---|---|
| Human growth | Radioimmunoassay | None | Expensive, | (Selden et al., |
| hormone (hGH) | | | insensitive, | 1986) |
| | | | narrow linear range. | |
| β-glucuronidase | Colorimetric, | colorimetric | sensitive, | (Gallagher, |
| (GUS) | fluorescent, or | (histo-chemical | broad linear | 1992) |
| | chemi- | staining with X- | range, non- | |
| | luminescent | gluc) | iostopic. | |
| Green | Fluorescent | fluorescent | can be used in | (Chalfie et al., |
| fluorescent | | | live cells; | 1994) |
| protein (GFP) | | | resists photo- | |
| and related | | | bleaching | |
| molecules (REP, | | | | |
| BFP, msx1, | | | | |
| etc.) | | | | |
| Luciferase | bioluminsecent | Bio- | protein is | (de Wet et al., |
| (firefly) | | luminescent | unstable, | 1987) |
| | | | difficult to | |
| | | | reproduce, | |
| | | | signal is brief | |
| Chloramphenicoal | Chromatography, | None | Expensive | (Gorman et al., |
| acetyltransferase | differential | | radioactive | 1982) |
| (CAT) | extraction, | | substrates, | |
| | fluorescent, or | | time- | |
| | immunoassay | | consuming, | |
| | | | insensitive, | |
| | | | narrow linear | |
| | | | range | |
| β-galacto-sidase | colorimetric, | colorimetric | sensitive, | (Alam and |
| | fluorescence, | (histochemical | broad linear | Cook, 1990) |
| | chemi- | staining with X- | range; some | |
| | luminscence | gal), bio- | cells have high | |
| | | luminescent in | endogenous | |
| | | live cells | activity | |
| Secrete alkaline | colorimetric, | None | Chemiluminscence | (Berger et al., |
| phosphatase | bioluminescent, | | assay is | 1988) |
| (SEAP) | chemiluminescent | | sensitive and | |
| | | | broad linear | |
| | | | range, some | |
| | | | cells have | |
| | | | endogenouse | |
| | | | alkaline | |
| | | | phosphatase | |
| | | | activity | |
| Tat from HIV | Mediates | Mediates | Exploits amino | (Frankel et al., |
| | delivery into | delivery into | acid residues | U.S. Pat. No. |
| | cytoplasm and | cytoplasm and | of HIV tat | 5,804,604, |
| | nuclei | nuclei | protein. | 1998) |

G. Biochemical

An extract is most simply a preparation that is in a different form than its source. A cell extract may be as simple as mechanically-lysed cells. Such preparations may be clarified by centrifugation or filtration to remove insoluble debris.

Extracts also comprise those preparations that involve the use of a solvent. A solvent may be water, a detergent, or an organic compound, as non-limiting examples. Extracts may be concentrated, removing most of the solvent and/or water; and may also be fractionated, using any method common to those of skill in the art (such as a second extraction, size fractionation by gel filtration or gradient centrifugation, etc.). In addition, extracts may also contain substances added to the mixture to preserve some components, such as the case with protease inhibitors to prolong protein life, or sodium azide to prevent microbial contamination.

Often, cell or tissue extracts are made to isolate a component from the intact source; for example, growth factors, surface proteins, nucleic acids, lipids, polysaccharides, etc., or even different cellular compartments, including Golgi vesicles, lysosomes, nuclei, mitochondria and chloroplasts may be extracted from cells.

III. Practicing the Invention

A. RNLE Extract

The following describes the preparation of a regenerating newt limb extract developed for the instant invention. Also see Examples. It will be apparent to one of skill in the art that many variations of the following procedure may yield extracts with similar activities. In general, any extract produced from newts that has at least one of the activities of the extract (see examples) is contemplated by the inventors.

However, any extract comprising regeneration activities can be similarly prepared from any animal that regenerates, for example, urodeles (newt or axolot1) and teleost fish, such as Danio rerio, (zebrafish), or from regenerating mammalian liver. Such extracts will have at least one activity of RE.

For example, adult newts, Notophthalmus viridescen.s are maintained in a humidified room. Operations are performed on anesthetized animals. Regenerating limb tissue is collected as follows. Forelimbs are amputated by cutting just proximal to the elbow and soft tissue is pushed up the humorus to expose the bone. The bone and soft tissue are trimmed to produce a flat amputation surface. The newts are placed in a sulfamerazine solution overnight and then back into a normal water environment. Early regenerating tissue (days 1, 3, and 5 postamputation) is collected by reamputating the limb 0.5-1.0 mm proximal to the wound epithelum and removing any residual bone. Nonregenerating limb tissue is collected from limbs that had not been previously amputated. Tissue is extracted 2-3 mm proximal to the forelimb elbow and all bones are removed. Immediately after collection, all tissues are flash frozen in liquid nitrogen and stored at −80° C.

Tissues are thawed and all subsequent manipulations are performed at 4° C. or on ice. Six grams of early regenerating tissue from days 1, 3, and 5 (2 grams each) or six grams of nonregenerating tissue are placed separately into appropriate cell culture medium containing three protease inhibitors (for example, leupeptin, A-protinin, and phenylmethysulfonyl fluoride). Tissues are ground with a tissue homogenizer, hand homogenized, and then briefly sonicated. Cell debris is removed in two centrifugation steps. The nonsoluble lipid layer is aspirated and the remaining supernatant filter sterilized. The protein content is then assayed and the extract stored at −80° C.

B. hRNLE; Identifying Active Components of RNLE

1. Introduction

The invention also comprises a composition that mimics at least one activity of RNLE that comprises human forms of the active molecules. For example, if Fgf is a component of RNLE (a likely possibility; see Examples), a human form of Fgf would be substituted in hRNLE compositions. A “humanized” formulation of RNLE would be advantageous to circumvent provoking an immune response in a human subject in need of a RNLE or RNLE-like composition.

2. Biochemical Approach

To one of skill in the art, it will be apparent how to determine the composition of hRNLE, using RNLE as a starting point and a functional assay based on, for example, regenerating newt limbs, or inducing dedifferentiation of mammalian myotubes. For example, using classic biochemical separation techniques, the components of RNLE can be fractionated and tested in a functional assay. When an activity is found, even if only a partial or subtle effect, then the isolated component is a candidate molecule that comprises an active RNLE. While each component may have a small effect, the sum of all RNLE purified active components will mimic that of RNLE.

3. Genetic Approach

To identify the active components in RNLE, and even the pathway and succession of events in regeneration, a genetic system can be employed. The invention demonstrates that fin regeneration in the genetically-amenable organism of Zebrafish requires Fgf signaling. Using a genetic approach, the individual genes that encode the factors responsible for RNLE-like activity can be identified by mapping and cloning. Once cloned, the Zebrafish gene sequences can be used to identify human homologues, using, for example cDNA or genomic DNA screening of human libraries. Similarly, BLAST searches and other in silico methods may obviate the need for such experimentation for some of the identified genes. In such a way, hRNLE (or that of the organism of choice) may be formulated.

The following outlines one genetic approach. However, one of skill in the art may vary or take a different genetic approach to achieve the same goal. For example, in cases where homozygosity at a mutated gene results in lethality, one of skill in the art may look for mutants with conditional alleles, such as temperature sensitive alleles. In general, a genetic approach requires a suitable organism, such as Zebrafish, and a screen or selection (a screen allows for the identification of a desired mutant among many other undesired mutants; a selection results in only the desired mutants). Fin regeneration in Zebrafish (see Examples) can be used as an easily-scored visual screen. Desirable mutants would be those individuals that either fail to completely regenerate a wild-type (wt) fin, those that regenerate a larger, but otherwise normal, fin, those that regenerate multiple fins, or those that grow back a different body part.

One of skill in the art would start such a screen by first mutagenizing a genetically-defined (pure) population of fish using methods well-known in the art. Mutagen cause various mutations in DNA sequences. Chemical mutagens, such as EMS and ENU, most often cause simple base-pair changes. More drastic mutagens include V, fast-neutrons, and X-rays, which can also cause base-pair changes, but also small and large deletions and chromosomal rearrangements. One of skill in the art will select a mutagen or mutagen(s) based on factors that include the organism of choice, the gene mapping technologies available, the desired types of mutations, and safety.

Once a population of mutagenized individuals is obtained, an initial screen for fin regeneration can be done in the Ml generation (the first generation after mutagenesis) to look for dominant mutations (those mutated genes that require only one copy to exert its phenotype). Fins would be amputated, and then screened for regenerative capacity, first visually, and if necessary, microscopically (but with live organisms). Dominant mutations, for the purposes of gene mapping and cloning, can be examined by using the wt phenotype as a recessive marker.

However, many mutations will be homozygous recessive. The M1 population is self-crossed (mated) so that homozygous loci are achieved in the M2 population. The screen for fin regeneration is repeated.

As mutant individuals are isolated, it is often desirable to “clean up” their genetic background, especially if many mutations were induced during mutagenesis (one of skill in the art will determine the rate of mutagenesis by, for example, examining a mutagenized population for a mutation). This step eliminates potential multi-gene defects, which are more difficult and potentially confusing to work with. To rid a mutant of “background” mutations, it is crossed with a wt individual (“back-crossed”). The progeny are then self-crossed (“selfed”), and the F2 generation is analyzed for the return of the mutant phenotype. Those lines wherein the mutant phenotype reappears are excellent candidates for further analysis. Preferably, these mutants are backcrossed a second time or more.

To identify the number of genes under examination, the mutants are crossed to each other to identify complementation groups. Complementation occurs when a wild-type phenotype is found in all of the F2 progeny. The simplest interpretation, with the caveat that complementation can occur (or not occur) in a minority of cases for multitudes of reasons, is that the mutated genes are not the same gene in the parents. If complementation does not occur, then this result usually indicates that the two parents have mutations in the same gene. Each complementation group indicates a single gene. All lines are maintained in each complementation group.

The mutated gene may then be mapped, using techniques well-known to those of skill in the art. The specifics of mapping, especially the use of linking-markers (whether, for example, morphological or DNA polymorphisms), are unique to the organism being studied. In one approach, mutant individuals are crossed to “mapping populations”—which have genetic markers that are well defined, either genetically or cloned—and mutant individuals are examined for the linkage of the mutant phenotype to the marker. Another very useful mapping population is a distantly related strain of the organism under study; wherein, for example, 1 in 10 bps, 1 in 100 bps, 1 in 1000, or 1 in 10,000 bps in the coding DNA sequences between the two strains differ. Such populations allow for the easy use of PCR-based markers which are exceptionally easy and quick to score.

When mapping becomes more and more fine, other techniques may be exploited to facilitate cloning the mutated gene. For example, if the region wherein the mutation falls has a known sequence, candidate genes can be identified. Such genes can then be sequenced in the mutant individuals to identify deleterious mutations (including changes in amino acid sequence or premature stop codons). If the region has an unknown sequence, cloning by phenotypic rescue can be exploited. The region in which the mutation falls can be isolated from wt individuals, broken into smaller pieces (enzymatically or by physical force), subcloned into appropriate expression vectors, and then transformed into mutant individuals. If the mutant phenotype is rescued—that is, the transformed individual regenerates a fin in the screening assay—then this is proof that the segment of DNA that was transformed carries the gene of interest. The introduced DNA can then be sequenced using well-known methods. In the case of dominant mutations, the mutant individual supplies the DNA, and the DNA pieces introduced into wt individuals and the mutant phenotype scored. Rescue is ideally confirmed in at least 2 different lines from each complementation group. In addition, sequencing all members at the candidate gene position is done to confirm that deleterious mutations occur in each line, indicating various alleles of the mutated gene. Noteworthy, however, are mutations that occur in operably-linked regions, such as promoters and enhancers, and those at splice-site junctions, which may be more difficult to identify by simple sequencing. One of skill in the art will know how to approach these issues.

Once the gene is in hand, the sequence can be used to design probes or primers to identify human (or any other creature) homologues. Human cDNA or genomic libraries may be exceptionally useful. PCR-based approaches may require only a human genome template. Alternatively, in silico experiments can be done to search for human homologues, such as BLAST searching. To confirm that human homologues have similar activities as the gene with which they were probed, the human sequence can be transformed into mutant individuals from the original screen and tested for mutant phenotype rescue. However, if that should fail, the human sequence can be subcloned into an expression vector, transformed into a suitable host (such as E. Coli, COS cells, or Drosophila S2 cells), expressed in vitro and harvested, and then applied to, for example, a cell dedifferentiation assay or myotube cleavage/proliferation assays, such as those described below (4 (e, f)).

4. Differential Gene Expression Approach to Identify hRNLE

In a first part, candidate genes that regulate cellular plasticity can be identified by employing both differential display analysis and by preparing a suppression subtractive cDNA library between early newt limb regenerates and nonregenerating limbs. Differential expression of the cloned cDNA fragments can be confirmed by dot blot hybridization or northern blot analysis. Full-length cDNA clones for selected candidate genes can be generated by screening a newt limb regeneration cDNA library. Such cDNA clones are then sequenced and full-length open reading frames identified.

In a second part, the sequences of candidate cellular plasticity genes are analyzed by computerized BLAST and motif searches to determine whether candidate cDNAs are homologues of known genes or if they possess interesting functional domains. The degree of upregulation following limb amputation can be assessed by Phosphorimage analysis of northern blots. Cellular expression patterns of the candidate genes can be determined by whole mount or tissue section in situ hybridization of the regenerating newt limb. Genes that show marked upregulation and contain domains usually found in growth factors, cytokines, or other ligands are likely candidates. Other genes of interest include metalloproteinases (enzymes that break down the extracellular matrix and could aid in cellular dedifferentiation), receptors (which could bind the ligands that initiate the dedifferentiation process), transcription factors (potential regulators of dedifferentiation genes or downstream response genes), and intracellular signaling molecules (could be involved in dedifferentiation or other regenerative processes).

In a third part, candidate genes are assayed for a role in initiating cellular dedifferentiation. In one approach, candidate genes are cloned into a mammalian expression vector and transfected into COS-7 cells. Conditioned media is collected from the transfected COS-7 cells and used to treat C2C12 myotubes. The myotubes are monitored over several days for signs of cellular dedifferentiation, such as reentry into the cell cycle, reduction in the levels of muscle differentiation proteins, and cell cleavage and proliferation. More than one protein may be required for the initiation of cellular dedifferentiation. Therefore, combinations of candidate genes can be assayed by cotransfecting more than one candidate gene into COS-7 cells, or by combining conditioned medium generated from transfections with different candidate genes. If the sequence and expression patterns of a particular candidate gene suggest that the protein it encodes may function intracellularly downstream of the initiating signals, the gene can be ectopically expressed in C2C12 myotubes to determine its ability to induce cellular dedifferentiation.

(a) Differential Expression Anaylsis Experimental Details

Total RNA is extracted from 30 regenerating newt limbs at 1, 3, and 5 days postamputation. Nonregenerating limb tissue is then collected from the same newts at the time of the initial amputation. Comparing regenerating and nonregenerating tissues from the same newts should eliminate any false positives in differentially-displayed cDNAs that are due to polymorphisms found in the wild newt population. The total volume of tissue is estimated and total RNA is isolated. Residual contaminating DNA is destroyed by treating the RNA with RNase-free DNasel, extracting the samples with phenol:chloroform:isoamyl alcohol and then precipitating with ethanol. RNA concentration and purity is determined by absorbance spectrophotometry at 260 nm and 280 mu. RNA integrity is assessed by running the samples on a 1% agarose gel in the presence of 0.5 M formaldehyde. Only nondegraded RNA is used for differential display analysis.

Differential display analysis is based on the differential reverse transcribed polymerase chain reaction (RT-PCR) amplification of RNA transcripts originating from genes that are expressed at different levels in the two tissues being compared. In one approach, reverse transcription is performed with anchor primers that bind to the poly(A) tract and are anchored by a single nucleotide (A, C, or G) on the 3′-end. Subsequent PCR amplifications are performed using the 3′-anchor primer and 1 of 80 different random primers designed to anneal to different sequences. Therefore, 240 different sets of primers are used to amplify the first-strand cDNA products. This approach provides nearly complete coverage of all transcripts expressed in the regenerating and nonregenerating newt limb. Differential display analysis is performed using regenerating and nonregenerating tissues collected at days 1, 3, and 5 postamputation. The amplified products are heat-denatured and separated on 0.4 mm 5% polyacrylamide/8M urea gels at 70 W for approximately 3 hours. The gels are dried, and Kodak X-ray BMR film is exposed for 12-16 hours. Reactions that produce differentially-displayed cDNA fragments is repeated using total RNA extracted from an independent set of tissues to confirm the differential display pattern.

The differentially-displayed cDNA fragments are excised from the dried gel and eluted by placing the gel in 100 all of TE (10 mM Tris-HCl, pH 7.5, 0.1 mM EDTA) and heating to 37° C. with constant shaking overnight. The Whatmann paper and gel debris are removed by centrifugation, and the cDNA-containing supernatant is saved for PCR amplification. Two amplification reactions are then performed. In the first reaction, 4 μl of undiluted cDNA eluate is used as template, and in the second reaction, the eluted cDNA is diluted 1/10 in TE and then used as template. The excised cDNAs are amplified by PCR, and the amplification products are separated on 1.8% low melting point agarose gels. The appropriate fragments are excised and gel purified. Purified fragments are ligated into a T/A cloning vector (such as pBluescript II SK), and transformed bacterial colonies are grown to isolate the plasmid DNAs. Recombinant plasmids are then used for making probes for northern blots and for sequence analysis.

Northern blot analyses are performed to confirm that differentially-displayed cDNA fragments represent genes that are truly differentially expressed between regenerating and nonregenerating tissue. Some differentially-expressed genes may be expressed at low levels and are not be detected using northerns prepared from total RNA. Therefore, differentially-displayed cDNAs using northerns prepared from single-selected poly(A) RNA from newt limbs are used. Northern blots are prepared by running 2 μg of nonregenerating limb and early limb regenerate poly(A) RNA (1, 3, and 5 days postamputation) in adjacent lanes. Ten sets of early limb regenerate/nonregenerating limb lanes are run. RNA is separated by electrophoresis at 80 V through 1% agarose gels containing 0.5 M formaldehyde, 20 mM MOPS, pH 7.0, 5 mM sodium acetate, and 1 mM EDTA. The RNA is blotted onto nylon membranes, UV-crosslinked to the membrane, and stained with 0.04% methylene blue in 0.5 M sodium acetate. The RNA is hybridized with cDNA probes prepared by random hexamer priming and ³²P-dCMP incorporation using inserts purified from recombinant plasmids. Differential expression is determined by comparing the intensity of the autoradiographic signals between lanes. Phosphorimage analysis is performed to quantitate the level of up- or down-regulation. Those exhibiting a 3-fold or greater transcriptional induction encode candidate active RNLE components.

(b) Suppression Subtractive cDNA Library Experimental Details

Candidate regeneration and dedifferentiation genes can also be identified by generating a suppression subtractive hybridization cDNA library using RNA isolated from early newt limb regenerates to prepare tester cDNA and RNA isolated from nonregenerating newt limbs to prepare the driver cDNA. Suppression subtractive hybridization is based on two important phenomena: (1) the ability of excess driver cDNA to effectively hybridize nearly all complementary cDNAs found in the tester cDNA population, leaving the unique tester transcripts as unhybridized single strands and (2) the ability of long inverted repeats located at opposite ends of the same cDNA molecule to anneal to each other and prevent primers from binding to the annealed ends.

Single-selected poly(A) RNA is isolated from total RNA that has been extracted from 200 regenerating newt limbs at 1, 3, and 5 days postamputation, and from 600 nonregenerating limbs as described above. A second round of poly(A) selection by binding the once-selected poly(A) RNA to the oligo(dT) cellulose matrix a second time, washing the cellulose, and eluting and concentrating the RNA as described above is performed.

First-strand cDNAs are prepared from both the experimental tester (early limb regenerates) and driver (nonregenerating limb) poly(A) RNAs. Two micrograms of poly(A) RNA are reverse transcribed at 42° C. for 1.5 hours using AMV reverse transcriptase. Second-strand cDNA synthesis is performed for 2 hours at 16° C. in the presence of DNA polymerase I, RNaseH, and E. coli DNA ligase. T4 DNA polymerase is added, and the samples incubated an additional 30 minutes at 16° C. Second-strand cDNA synthesis is terminated by adding an EDTA/glycogen mix, and the samples are extracted with phenol:chloroform:isoamyl alcohol and chloroform and precipitated with ethanol. The cDNAs are resuspended in ddH₂O, digested with RsaI, and purified by phenol:chloroform extraction and ethanol precipitation.

The purified RsaI-digested cDNAs from the regenerating limb are divided into two aliquots. Adaptor 1 is ligated to the cDNA ends of one of these aliquots and Adaptor 2R is ligated to the cDNA ends of the second aliquot. Adaptor-ligated cDNAs from the regenerating limb (adaptor 1-ligated and adaptor 2R-ligated) are mixed separately in two different vials with a 30-fold excess of cDNA (lacking adaptors) from the nonregenerating limb. These samples are denatured at 98° C. for 1.5 minutes and then allowed to anneal at 68° C. for 6-12 hours. The two cDNA samples from the regenerating limb that contain different adaptors are then be mixed together with freshly denatured cDNA from the nonregenerating limb (no adaptors) and annealed overnight at 68° C. Following this second round of hybridization, the single-stranded 5′-ends are filled-in using a thermostable DNA polymerase and dNTPs, and then the hybridized products are subjected to 27 cycles of suppression PCR using a primer specific for both adaptors. The PCR products are then diluted and subjected to nested PCR using a primer that is specific for adaptor 1 and a second primer specific for adaptor 2R. During these steps, templates that have the same adaptor on both ends are not be efficiently amplified, because the two ends of each template contain long stretches of complementary base pairs that anneal to each other and form hairpin loops that prevent primers from reaching their target sequences. The amplified cDNA products are then ligated into T/A cloning vectors (such as pBluescript II SK) to construct a library consisting primarily of cDNAs that are preferentially expressed in the early regenerating limb. The same procedure can be followed to produce a library of cDNAs that are preferentially expressed in the nonregenerating limb.

Although this procedure enriches for differentially expressed genes, it can produce false positives. To confirm differential expression, dot blot analysis by probing filters containing subtracted cDNA clones from the regenerating limb with either labeled cDNAs from the subtracted regenerating limb or from the subtracted nonregenerating limb are performed. Clones that show differential hybridization patterns when probed with these two cDNA populations are selected for confirmation of differential expression by northern blot and Phosphorimage analysis. The inserts of confirmed clones are then sequenced using established protocols well known in the art.

(c) Generation and Sequencing of Full-length Differentially Expressed cDNAs Experimental Details

The following protocol can be used to identify full-length human cDNAs, using human cDNA libraries. Stringency conditions may need to be adjusted (Ausubel et al., 1987).

Full-length cDNA clones are generated for selected cDNAs by screening the newt early limb regenerate cDNA library using a probe made from either the original differentially-displayed cDNA fragment or the subtracted cDNA. Probes are labeled by random hexamer priming and incorporation of ³²P-CMP. One million cDNAs cloned into a phage vector are plated at high density, and duplicate lifts onto nylon membranes prepared. The membranes are hybridized with the ³²P-labeled cDNA probes. Secondary screens are performed by selecting the positive plaques and then replating them at a density of 300-500 plaques per 150 mm plate. Plaques are lifted onto nylon membranes and hybridized with the specific cDNA probes. Isolated positive plaques from the secondary screen are selected and grown. The cDNA inserts are excised in vivo as pBK-CMV plasmid constructs with RE704 helper phage, and the clones selected on agar with 50 μg/ml kanamycin. Colonies are selected, grown in LB-kanamycin culture, and plasmids isolated. The clones are then digested with EcoRI and XhoI to excise the cDNA inserts, and the digests separated on 1% agarose gels to determine insert sizes. The insert size for each clone is compared to its corresponding transcript size as determined by northern blot analysis to assess whether the clone might contain full-length cDNA. The ends of the clones are sequenced. If a cDNA clone is not full-length, probes are designed from either the 5′- or 3′-end or both (depending on which end of the cDNA is missing) and the library screened again. This process is reiterated until the full-length open reading frame is obtained. In cases where screening the library fails to identify a full-length open reading frame, 5′ or 3′ RACE (Rapid Amplification of cDNA Ends) can be used to clone the missing portion of the cDNA.

(d) Selection of Candidate Cellular Plasticity Genes Based Upon Sequence Analysis, Level of Upregulation, and Cellular Expression Patterns.

Sequence Analysis of Differentially Expressed cDNAs cDNA sequences of differentially expressed genes are analyzed by nucleotide and protein BLAST searches (Altschul and Gish, 1996; Altschul et al., 1997). Not every candidate cellular plasticity gene will be recognized as belonging to a particular gene family. These novel genes could play important roles in cellular plasticity, and those that exhibit a significant transcriptional induction following amputation are tested for function (see below).

Riboprobe Synthesis Riboprobes are used in whole-mount and tissue section in situ hybridization procedures. These probes are labeled with digoxigenin (DIG), which can later be detected with an anti-DIG antibody conjugated to alkaline phosphatase. Vector constructs containing the cDNA inserts are linearized by digestion with either BamHI for use as templates for T7 RNA polymerase or XhoI for use as templates for T3 RNA polymerase. Riboprobe synthesis is carried out as follows: Briefly, 1 μg of linearized cDNA-containing vector is used as template in a reaction containing DIG labeling mix, T3/T7 RNA polymerase transcription buffer, RNase inhibitor, and T3 or T7 RNA. Transcription is carried out at 37° C. for 2 hours. DNA is destroyed by the addition of DnaseI, and the riboprobes are purified by two successive ethanol precipitation steps. Following the final precipitation, the riboprobes are resuspended in ddH₂O treated with diethyl pyrocarbonate (DEPC) and the concentration and purity is determined by spectrophotometry at 260 and 280 nm. A 1% agarose gel is run in 1× TAE to confirm the presence and concentration of the riboprobes.

Preparation of Newt Limb Powder Newt limb powder is required to block alkaline phosphatase-conjugated anti-DIG antibody during the whole-mount in situ hybridization procedure. Use of newt powder to block the antibody reduces background staining due to nonspecific binding of the antibody to newt tissues. Amputated newt limbs are flash frozen in liquid nitrogen and stored at −80° C. until used to prepare newt limb powder. The frozen limbs are crushed into powder over liquid nitrogen using a mortar and pestle. The limb powder is treated with 4 volumes of ice cold acetone,-mixed, and placed on ice for 30 minutes. Following centrifugation, the acetone is removed, the sample rinsed with acetone, and transferred to a piece of Whatmann paper, where it is ground into a fine powder. After complete air drying, the limb powder is stored in an airtight container at 4° C.

Whole-Mount in situ Hybridization Whole-mount in situ hybridization on early limb regenerates (days 1-5) is performed to determine the expression patterns of the candidate cellular plasticity genes. Photographs of the stained whole-mount regenerates are taken and the tissues can then be sectioned. Analysis of the whole-mounts before sectioning allows for the assessment of the overall expression patterns of the genes, while analysis of the tissue sections reveals specific cellular expression patterns.

Newt limb amputations are performed as described above. The limbs are reamputated within 5 days of the initial amputation, and the tissue is fixed immediately in 3.7% buffered paraformaldehyde. The tissues are thoroughly washed with phosphate buffered saline containing 0.1% Tween 20 (PBST), dehydrated in a series of methanol/PBST and solutions, and then stored −20° C. in 100% methanol. Tissues are rehydrated in methanol/PBST solutions and then washed three times in PBST. The samples are treated with 20 μg/ml proteinase K at 37° C. for 10, 20, or 30 minutes. The tissues is then washed thoroughly with PBST at 4° C. to eliminate proteinase K activity and will be acetylated with 0.5% acetic anhydride in 0.1 M triethanolamine (pH 7.9) for 10 minutes. The tissues are washed with PBST and refixed for 20 minutes with 4% paraformaldehyde. The samples are washed thoroughly with PBST, washed in hybridization solution (50% formamirde, 5× SSC, 1 mg/ml yeast tRNA, 100 μg/ml sodium heparin, 1× Denhardt's solution, 0.1% Tween-20, 0.1% CHAPS, and 5 mM EDTA) and then prehybridized in a rotating hybridization oven overnight at 60-65° C. in hybridization solution. The riboprobes prepared above are heated to 95° C. for 30 minutes and added to the limb tissues at a concentration of 1 μg/ml. Hybridization is carried out for 48-72 hours at 60-65° C. To remove unbound riboprobe, the tissues are washed in hybridization solution for 20 minutes at 65° C., followed by three washes in 2× SSC at 65° C. for 20 minutes each and two washes in 0.2× SSC at 65° C. for 30 minutes each.

Hybridized probes are detected by washing the samples in MAB (100 mM maleic acid, 150 mM NaCl, pH 7.5) and then in MAB-B (MAB containing 2 mg/ml BSA). The tissues are treated with antibody blocking solution (20% heat-inactivated sheep serum in MAB-B) overnight at 4° C. At the same time, the alkaline phosphatase conjugated anti-digoxigenin antibody (Roche, Boehringer-Mannheim) is diluted 1:400 in blocking solution and preabsorbed overnight at 4° C. with 10 mg/ml newt limb powder. After preabsorption, the newt powder is removed by centrifugation, and the antibody is diluted to 1:1000 (an additional 2.5-fold dilution) in blocking solution and added to the tissue samples. Antibody incubation proceeds overnight at 4° C. Tissues are washed 10 times with MAB at room temperature (30 minutes each wash) and then washed twice in AP buffer (100 mM Tris-HCl, pH 9.5, 100 mM NaCl, 50 mM MgCl₂). The tissues are incubated in the alkaline phosphatase substrate NBT/BCIP in AP buffer containing 1 mM levamisole) for 1-6 hours in the dark. The tissues are washed several times in PBST and then postfixed overnight in buffered 4% paraformaldehyde. Samples are washed once in 70% ethanol and then stored in methanol at −20° C. Tissues are cleared in a 1:2 benzyl alcohol:benzyl benzoate solution (BABB). The whole-mount tissues are photographed to determine overall expression of the gene.

Following whole-mount in situ hybridization and photography, the cellular expression patterns are assessed by embedding the tissues in paraffin and sectioning the blocks at 12-20 μm. Tissue sections are examined and photographed.

In situ Hybridization of Tissue Sections If the whole-mount procedure produces a chromogenic signal that is too weak to decipher, in situ hybridization on tissue sections can be performed. Following amputation, tissues are frozen directly in OCT. The tissues are sectioned with a cryostat at 10 μm and fixed for 1 hour in 4% paraformaldehyde DEPC-PBS. The slides are washed in 2× SSC (DEPC-treated) and then treated with 0.2 M HCl for 8 minutes. The tissues are rinsed with 0.1 M triethanolamine (pH 7.9) and acetylated with 0.25% acetic anhydride in 0.1 M triethanolamine for 15 minutes. The slides are washed with 2× SSC and heat-denature riboprobe (80° C., 3 minutes) in hybridization solution (50% formamide, 4× SSC, 1× Denhardt's solution, 500 μg/ml heat denatured herring sperm DNA, 250 μg/ml yeast tRNA, and 10% dextran sulfate) are added to the tissue sections. Cover slips are sealed over the tissues and hybridization are carried out overnight at 55° C. in a humidified chamber. The tissues are washed in 2× SSC, then in STE (500 mM NaCl, 20 mM Tris-HCl, pH 7.5, and 1 mM EDTA), and treated with RNase A (40 μg/ml in STE) for 30 minutes at 37° C. Sections are washed with 2× SSC, 50% formamide at 55° C., then with 1× SSC at room temperature, and finally with 0.5× SSC at room temperature.

Bound riboprobes are detected by washing the slides for 1 minute in Buffer 1 (100 mM Tris-HCl, pH 7.5, 150 mM NaCl), then blocking the tissues with 2% sheep serum in Buffer 1. Sheep anti-digoxigenin antibody conjugated to alkaline phosphatase (Roche) is diluted 1:500 in Buffer 1 containing 1% sheep serum, added to the tissues, and incubated in a humidified chamber at room temperature for 1 hour. Slides are then washed in Buffer 1, followed by a wash in Buffer 2 (100 mM Tris-HCl, pH 9.5, 100 mM NaCl, 50 mM MgCl₂). Substrate solution (NBT/BCIP in Buffer 2 with 1 mM levamisole) is added to the sections and the slides incubated in the dark at 4° C. overnight. The reaction is terminated by placing the slides in Buffer 3 (10 mM Tris-HCl. pH 8.0, 1 mM EDTA). The tissues are mounted and observed for chromogenic staining by light microscopy.

Prioritizing Candidate Cellular Plasticity Genes Candidate cellular plasticity genes can be prioritized according to their gene families, degree of transcriptional induction, and cellular expression patterns. Genes that are significantly upregulated and encode potential extracellular signaling molecules, such as growth factors, cytokines, or other ligands, are immediate candidates. Such genes may encode factors that initiate the cellular dedifferentiation of the internal stump cells. Other genes of primary interest include receptors, which could bind the initiating ligands, kinases, which could play a role in the intracellular transduction of the dedifferentiating signals, and transcription factors, which could be response genes that either induce or repress downstream genes involved in dedifferentiation or maintenance of the differentiated state. Metalloproteinases could be involved in cellular dedifferentiation by interrupting the extracellular matrix. Finally, novel genes that are markedly upregulated following amputation but do not belong to any known gene family are of interest, because they could function in regulating cellular plasticity.

Between 30-100 differentially-expressed genes can be expected from this approach, of which up to 50% of the genes are likely to be mitochondrial genes, general cell cycle genes, or other housekeeping genes and therefore unlikely RNLE components. The remaining candidate genes are then tested for function in initiating or inducing cellular dedifferentiation as described below.

(e) Assay to Determine if Candidate Genes Play Roles in Cellular Plasticity

The differentially-expressed genes that are candidates for regulating cellular plasticity are then tested to determine whether they function to induce cellular dedifferentiation in cultured mouse C2C12 myotubes, or in another embodiment, dedifferentiation of in vitro cultured human cells. Mouse myotubes can be induced to dedifferentiate either when treated with protein extracts from early limb regenerates (days 1-5 postamputation) or when induced to ectopically express msx1 in the presence of growth factors. Using a similar approach can determine whether a candidate gene induces cellular dedifferentiation. If the candidate gene appears to encode a secreted protein (possibly a growth factor, cytokine, or other ligand), it is cloned into an expression vector and determined whether treating mouse myotubes with the expressed protein can induce cellular dedifferentiation. If the gene appears to encode a cellular factor and is expressed in the underlying stump tissue, it is cloned into a mammalian expression vector and its expression induced in mouse myotubes and then determined whether the ectopic expression of the gene can induce mouse myotubes to dedifferentiate. If a single gene is unable to induce dedifferentiation, combinations of the various candidate genes are tested for their ability to induce cellular plasticity. If combinations of genes are unable to induce cellular plasticity, nonregenerating limb extracts are prepared, and then determine whether these extracts (which do not induce dedifferentiation on their own), in combination with the candidate genes, can induce dedifferentiation.

Testing Candidate Newt Genes for Their Ability to Initiate Dedifferentiation of Mouse Myotubes Genes whose sequences suggest they may be secreted soluble factors will be tested for their ability to initiate cellular dedifferentiation of mouse myotubes. A relatively easy approach to determine whether a secreted gene can initiate cellular dedifferentiation is to transfect cultured COS-7 cells with a plasmid construct containing the candidate gene driven by a mammalian promoter, such as a CMV promoter. A few days following transfection, the cell culture medium is collected. Secreted soluble proteins expressed in the COS-7 cells are present in this conditioned medium. The conditioned medium can then be used to treat terminally-differentiated mouse myotubes or cultured human cells to determine whether the expressed protein can initiate the dedifferentiation process. Controls consist of conditioned medium from mock-transfected COS-7 cells.

A single candidate gene may not be able to initiate cellular dedifferentiation, while combinations of candidate genes may induce such a response. Therefore, if no single gene can initiate dedifferentiation on its own, cotransfection of combinations of candidate dedifferentiation genes into COS-7 cells are performed and then determine whether the resulting conditioned medium can induce cellular dedifferentiation. Alternatively, conditioned medium from singly-transfected COS-7 cells can be combined and the dedifferentiation assays performed using the combined medium.

Transfection of COS-7 cells and Confirmation of the Presence of Candidate Proteins in Conditioned Medium COS-7L cells are grown and passaged in DMEM containing 0.1 mM nonessential amino acids (NEAA) and 10% FBS at 37° C. in 5% CO₂. The day before transfection, 2×10⁶ cells are plated in 12 ml of growth medium on 100 mm poly-D-lysine-coated tissue culture plates. A hemagglutinin tag is added to the 3′-end of the full-length cDNAs so that the presence of protein in the conditioned medium can be ascertained. The entire construct is cloned into the pBK-CMV expression vector and transfected into cultured COS-7L cells using liposome-mediated transfection. Conditioned medium is collected to use in dedifferentiation assays 48 hours after the initiation of transfection.

The conditioned medium is tested for the presence of the candidate dedifferentiation protein using Western blot analysis. Proteins are separated on 4-20% linear gradient gels and then transferred to nylon membranes by electrophoresis. The membranes are air dried, blocked with 5% nonfat dry milk, and then incubated overnight at 4° C. in a solution containing anti-hemagglutinin antibody (mono HA. 11, BabCo) diluted 1:1000 in blocking solution. The blots are thoroughly washed and incubated for 1 hour with a peroxidase-conjugated anti-mouse IgG secondary antibody diluted 1:1000 with blocking solution. The blots is thoroughly washed and enhanced chemiluminescence is performed to determine whether the candidate dedifferentiation protein is present in the conditioned medium.

Testing Candidate Proteins for Their Ability to Induce Cell Cycle Reentry

To determine whether a candidate protein can induce mouse myotubes to reenter the cell cycle, BrdU-incorporation experiments are performed. Briefly, C2C12 myoblasts (or cultured human cells) are grown to confluency in 24-well plates in growth medium (GM—20% FBS and 4 mM glutamine in DMEM) and then induced to differentiate by replacing GM with differentiation medium (DM—2% horse serum and 4 mM glutamine in DMEM). The myocytes are allowed to differentiate for 4 days. C2C12 myotubes in different wells are then be treated with different dilutions of the conditioned medium (undiluted, ½, ¼, ⅛, 1/16, and a control well with no conditioned medium) for up to 4 days. BrdU is added to the cultures at a concentration of 10 mmol/ml 12 hours before testing for cell cycle reentry. BrdU incorporation is assayed using the 5-bromo-2′-deoxy-uridine labeling. Briefly, the cells are thoroughly washed with PBS, fixed for 20 minutes at −20° C. with 70% ethanol/15 mM glycine buffer (pH 2.0), and washed again. Cells are then incubated in a 1:10 dilution of anti-BrdU antibody for 30 minutes at 37° C. The cells are washed and then incubated in fluorescein-conjugated anti-mouse IgG for 30 minutes at 37° C. After washing, the cells are observed microscopically and photographed using a FITC filter. Cells containing nuclei that fluoresce green have incorporated BrdU during DNA synthesis and are regarded as having reentered the cell cycle. Given that cell cycle reentry plays an important role in cellular dedifferentiation, any candidate newt gene that induces reentry into the cell cycle is considered to be an important gene for the initiation of cellular dedifferentiation and plasticity.

Testing Candidate Proteins for Their Ability to Reduce Levels of Muscle Differentiation Proteins To determine whether a candidate gene can reduce the levels of muscle differentiation proteins, mouse myotubes (or cultured human muscle cells) as described above are treated with the conditioned medium from COS-7L cells expressing the candidate gene. After 3 days of treatment, immunofluorescent assays are performed to determine whether there has been a reduction in the levels of MyoD, myogenin, MRF4, troponin T, and p21. MyoD, myogenin, and MRF4 are important regulators of myogenesis, while p21 signals the onset of the postmitotic state and troponin T is a component of the contractile apparatus. All of these factors are normally expressed in C2C12 myotubes, and a reduction in their levels signify a reversal in cell differentiation. The cells are washed with PBS, fixed in Zamboni's fixative for 10 minutes, washed again with PBS, and permeabilized with 0.2% Triton-X-100 in DPBS for 20 minutes. The cells are blocked with 5% skim milk in DPBS for 1 hour at room temperature and then exposed to the primary antibodies overnight at 4° C., using primary antibodies that recognize MyoD, myogenin, MRF4, troponin T. and p21. The cells are washed and then treated for 45 minutes at 37° C. with either goat anti-rabbit IgG conjugated to Alexa 488, goat anti-mouse IgG conjugated to biotin, or both secondary antibodies, depending upon the primary antibody(ies) used. The cells are washed and then either observed fluorescently or treated with streptavidin-Alexa 594 for 45 minutes at 37° C. The latter cells are washed and then observed with fluorescent microscopy using FITC and Texas Red filters. Cell nuclei are visually observed to determine whether the levels of the myogenic regulatory factors MyoD, myogenin, and MRF4, and p21 have been reduced. Cytoplasm is observed to determine whether troponin T levels are reduced. Reduced levels of these muscle differentiation proteins are another indicator of myotube dedifferentiation. For controls, cells not treated with conditioned media are used. Therefore, any candidate gene that can induce these cellular changes are considered an important gene for the initiation of cellular dedifferentiation and plasticity.

Testing Candidate Proteins for Their Ability to Induce Myotube Cleavage and Cell Proliferation Any candidate gene that initiates reentry into the cell cycle and/or reduction in muscle differentiation protein levels is tested for its ability to induce cell cleavage and proliferation. Myotubes (or human muscle cells) are generated as described above, except large numbers are plated on 100 mm tissue culture plates. These cells are purified and replated at low density. Residual mononucleated cells are eliminated by needle ablation and lethal water injections. The cells are photographed, conditioned medium is added, and the cells monitored by visual inspection and photography for up to 7 days. Cell culture medium containing conditioned medium is changed daily. Cleavage of myotubes to form smaller myotubes or proliferating, mononucleated cells are considered an indication of cellular dedifferentiation. Any candidate gene that can initiate myotube cleavage is considered an important gene for cellular dedifferentiation and plasticity.

(f) Testing Candidate Genes that Encode Cellular Proteins for a Possible Role in Dedifferentiation

Candidate genes that are expressed in the underlying stump and appear to encode cellular proteins, e.g. receptors, transcription factors, or signal transduction proteins are tested for a possible role in cellular dedifferentiation by ectopically expressing them in mouse (or human) myotubes. A retroviral construct (LINX) containing a doxycycline-suppressible candidate gene is transfected into Phoenix-Amphotropic cells using the CaPO₄ method, and the resulting recombinant retroviruses are harvested by saving the conditioned medium. Myoblasts are infected with the recombinant retrovirus by adding the conditioned medium to the myoblasts in the presence of 4 μg/ml Polybrene and allowing the infection to occur for 12-18 hours. The infection medium is replaced with myoblast growth medium containing 2 μg/ml doxycycline to prevent the expression of the candidate gene. The cells are allowed to grow for 48 hours, sub-cultured, and grown in the presence of 2 μg/ml doxycycline and 750 μg/ml G418 to select for transduced myoblasts. Selection continues for 14 days, and clonal populations are derived. Candidate genes are induced following myotube formation in the expanded clones by replacing DM-dox with medium lacking dox. The cells are then tested for reentry into the cell cycle, reduction in muscle differentiation proteins, and cell cleavage and proliferation as described above. A candidate gene that induces any of these indicators of cellular dedifferentiation is considered an important response gene in the cellular dedifferentiation pathway.

Alternatively, another approach may include the purification of candidate proteins expressed in either bacterial or eukaryotic cells. These purified proteins could then be used at specified concentrations in the cellular dedifferentiation assays described in this proposal.

5. Making and Using Antibodies to Identify Active RNLE Components

Because RNLE active components are likely proteins, polypeptides or peptides (see Examples), an antibody approach can be taken, especially if genetic or differential display approaches become difficult or nonproductive.

In this approach, antibodies are raised against antigens in whole RNLE, or in fractions of RNLE, in a host of choice. Preferably, the host is one from which monoclonal antibodies mAbs can be eventually derived. Once antibodies are produced, they are tested, first in vitro, then in vivo, for their ability to block a RNLE-dependent process, such as myotube dedifferentiation or newt limb regernation. Such antibodies can then be used to isolate human (or any other organism) homologues using a variety of approaches, such as screening human expression libraries, isolating the antigen-containing polypeptides by antibody affinity chromatography and performing terminal peptide sequencing and using such a sequence to perform in silico experiments or to design nucleic acid probes and primers to isolate nucleic acids encoding the corresponding polypeptides.

“Antibody” (Ab) comprises single Abs directed against an RNLE (anti-RNLE Ab; including agonist, antagonist, and neutralizing Abs), anti-RNLE Ab compositions with poly-epitope specificity, single chain anti-RNLE Abs, and fragments of anti-RNLE Abs. A “monoclonal antibody” is obtained from a population of substantially homogeneous Abs, i.e., the individual Abs comprising the population are identical except for possible naturally-occurring mutations that may be present in minor amounts. Abs include polyclonal (pAb), monoclonal (mAb), humanized, bi-specific (bsAb), and heteroconjugate Abs.

The following outlines one variation of this approach. One of skill in the art may choose other variations, or deviate from the following but will still achieve the same endpoint.

Newt limb extract is prepared as above (III. A.), in large quantity. Preferably, the extract is concentrated to minimize the aqueous component, such as by dialysis. Alternatively, the proteins may be isolated by any method known in the art, such as, for example, ammonium sulfate or trichloroacetic acid precipitation. This preparation is used as the antigen.

(a) Polyclonal Abs (pAbs)

Polyclonal Abs can be raised in a mammalian host, for example, by one or more injections of an immunogens (RNLE) and, if desired, an adjuvant. Typically, the immunogen and/or adjuvant are injected in the mammal by multiple subcutaneous or intraperitoneal injections. Examples of adjuvants include Freund's complete and monophdsphoryl Lipid A synthetic-trehalose dicorynomycolate (MPL-TDM). To improve the immune response, an immunogen may be conjugated to a protein that is immunogenic in the host, such as keyhole limpet hemocyanin (KLH), serum albumin, bovine thyroglobulin, and soybean trypsin inhibitor. Protocols for antibody production are well-described (Ausubel et al., 1987; Harlow and Lane, 1988). Alternatively, pAbs may be made in chickens, producing IgY molecules (Schade et al., 1996).

(b) Monoclonal Abs (mAbs)

Anti-RNLE mAbs may be prepared using hybridoma methods (Milstein and Cuello, 1983). Hybridoma methods comprise at least four steps: (1) immunizing a host, or lymphocytes from a host; (2) harvesting the mAb secreting (or potentially secreting) lymphocytes, (3) fusing the lymphocytes to immortalized cells, and (4) selecting those cells that secrete the desired (anti-RNLE) mAb.

A mouse, rat, guinea pig, hamster, or other appropriate host is immunized to elicit lymphocytes that produce or are capable of producing Abs that will specifically bind to the immunogen. Alternatively, the lymphocytes may be immunized in vitro. If human cells are desired, peripheral blood lymphocytes (PBLs) are generally used; however, spleen cells or lymphocytes from other mammalian sources are preferred The immunogen typically includes an RNLE or a fusion protein.

The lymphocytes are then fused with an immortalized cell line to form hybridoma cells, facilitated by a fusing agent such as polyethylene glycol (Goding, 1996). Rodent, bovine, or human myeloma cells immortalized by transformation may be used, or rat or mouse myeloma cell lines. Because pure populations of hybridoma cells and not unfused immortalized cells are preferred, the cells after fusion are grown in a suitable medium that contains one or more substances that inhibit the growth or survival of unfused, immortalized cells. A common technique uses parental cells that lack the enzyme hypoxanthine guanine phosphoribosyl transferase (HGPRT or HPRT). In this case, hypoxanthine, aminopterin and thymidine are added to the medium (HAT medium) to prevent the growth of HGPRT-deficient cells while permitting hybridomas to grow.

Preferred immortalized cells fuse efficiently, can be isolated from mixed populations by selecting in a medium such as HAT, and support stable and high-level expression of antibody after fusion. Preferred immortalized cell lines are murine myeloma lines, available from the American Type Culture Collection (Manassas, Va.). Human myeloma and mouse-human heteromyeloma cell lines also have been described for the production of human mAbs (Kozbor et al., 1984; Schook, 1987).

Because hybridoma cells secrete antibody extracellularly, the culture media can be assayed for the presence of mAbs directed against an RNLE (anti-RNLE mAbs). Immunoprecipitation or in vitro binding assays, such as radio immunoassay (RIA) or enzyme-linked immunoabsorbent assay (ELISA), measure the binding specificity of mAbs (Harlow and Lane, 1988; Harlow and Lane, 1999), including Scatchard analysis (Munson and Rodbard, 1980).

Anti-RNLE mAb secreting hybridoma cells may be isolated as single clones by limiting dilution procedures and sub-cultured (Goding, 1996). Suitable culture media include Dulbecco's Modified Eagle's Medium, RPMI-1640, or if desired, a protein-free or -reduced or serum-free medium (e.g., Ultra DOMA PF or HL-1; Biowhittaker; Walkersville, Md.). The hybridoma cells may also be grown in vivo as ascites.

The mAbs may be isolated or purified from the culture medium or ascites fluid by conventional Ig purification procedures such as protein A-Sepharose, hydroxylapatite chromatography, gel electrophoresis, dialysis, ammonium sulfate precipitation or affinity chromatography (Harlow and Lane, 1988; Harlow and Lane, 1999).

The mAbs may also be made by recombinant methods (U.S. Pat. No. 4,166,452, 1979). DNA encoding anti-RNLE mAbs can be readily isolated and sequenced using conventional procedures, e.g., using oligonucleotide probes that specifically bind to murine heavy and light antibody chain genes, to probe preferably DNA isolated from anti-RNLE-secreting mAb hybridoma cell lines. Once isolated, the isolated DNA fragments are sub-cloned into expression vectors that are then transfected into host cells such as simian COS-7 cells, Chinese hamster ovary (CHO) cells, or myeloma cells that do not otherwise produce Ig protein, to express mAbs The isolated DNA fragments can be modified, for example, by substituting the coding sequence for human heavy and light chain constant domains in place of the homologous murine sequences (U.S. Pat. No. 4,816,567, 1989; Morrison et al., 1987), or by fusing the Ig coding sequence to all or part of the coding sequence for a non-Ig polypeptide. Such a non-Ig polypeptide can be substituted for the constant domains of an antibody, or can be substituted for the variable domains of one antigen-combining site to create a chimeric bivalent antibody.

i. Screening for Function-Blocking Antibodies

If function-blocking antibodies are desired, screening hybridoma supernatants in pools represents an attractive option. Before limiting dilution to single cells, hybridomas after fusion are instead split into pools contains 2 to thousands of cells, representing 2 or more different antibodies. These supernatants, or prepations thereof, can be used to screen for their ability to inhibit RNLE-like activity in any of the assays outlined above (4 (e, f)), such as myotube dedifferentiation; or preferably, inhibit the ability of newt limbs to regenerate. Those pools that exhibit function-blocking activity are then subcloned by dilution into smaller pools, the screen repeated, and dilution of active pools repeated. This process is reiterated until clonal hybridoma cell lines are achieved. Function-blocking, in this case, does not necessarily indicated total inhibition of function; any antibody that shows an effect that is contrary to the activity of RNLE is a candidate.

Once such clonal lines are achieved, the antibodies can be used to isolate the polypeptides they bind, and identification of human or other animals homologues can proceed.

ii. Identification of Human Components of RNLE

The antibodies identified above can be used to affinity-purify the antigen-containing polypeptide. Once the polypeptides are isolated, they can be analyzed in a number of ways, known to those of skill in the art, to determine their sequence, for example N-terminal sequencing. Once a peptide fragment sequence is known, that sequence can be used to identify identical or similar proteins using protein-protein BLAST searches, or in the design of nucleic acid primers and probes. Such probes, which are degenerate due to the degeneracy of the genetic code, can be used to identify candidate nucleic acid molecules encoding homologues of the antibody antigen. Any appropriate library, or genome, may be screened. Preferably, a cDNA library is screened; most preferably, a cDNA library from human is screened.

Alternatively, the antibodies themselves may be used to directly identify similar or identical proteins from other species. For example, an expression library, preferably from human, may be screened with the antibodies. When binding is observed, that signal indicates a candidate human homologous protein. Alternatively, panning approaches or affinity chromatography may be exploited if protein misconformations prevent antibody binding of proteins produced in a bacterial-mediated expression library.

6. Candidate Approach

The inventors believe that the polypeptides, or their homologues, listed in Table C1 are likely components of RE.

TABLE C1
Candidate RE components

| Extracellular | Intracellular |
|---|---|
| Family members of Fibroblast | msx1 |
| Growth Factors (Fgfs) | |
| Family of Bone | msx2 |
| Morphophenetic Proteins | |
| (BMPs) | |
| Wnt proteins | E2F |
| Metalloproteinases | Fgf receptors |
| | BMP receptors |
| | frizzled (wnt receptors) |
| | SMADs (mothers against decapentaplegic) |
| | fatty acid binding Proteins |

Various approaches can be used to identify if the candidate components are active in RE. A skilled artisan will choose the approach.; For example, anti-sense or aptamers approaches can be used to inhibit expression of the intracellular candidate components in regenerating newt limb, using technology well-known in the art, and then testing the ability for the limb to regenerate. Alternatively, function-blocking antibodies that are available in the art against the various components can be used to inhibit newt limb regeneration. If the limb fails to fully differentiate, then the component is likely to be contained in RE.

C. msx1

The invention provides methods for cellular dedifferentiation and regeneration that use msx1. Because msx1 is an intracellular factor, it must be introduced into cells. Three methods are contemplated: (1) nucleic acid and gene therapy approaches, wherein msx1 is subcloned into a nucleic acid vector and then delived by another vector (such as adenovirus) or directly to the cells of interest; (2) a fusion msx1 polypeptide, wherein msx1 is fused to a polypeptide that usually gains entry to cells, such as HIV tat protein (see Table C); delivery can be affected by incorporation into a suitable pharmaceutical composition; and (3) incorporation of msx1 into a composition that is taken up by cells, such as in liposomes. Details of pharmaceutical compositions and their use can be found in herein.

While the following section pertains to msx1 gene therapy and molecular manipulation, the methods are applicable to other parts of the invention that also use nucleic acids, such as in the production of hRNLE by differential expression, etc.

1. Gene Therapy Compositions

The msx1 nucleic acid molecule (or a nucleic acid molecule encoding any active RDF component) can be inserted into vectors and used as gene therapy vectors. Gene therapy vectors can be delivered to a subject by, for example, intravenous injection, local administration (Nabel and Nabel, U.S. Pat. No. 5,328,470, 1994), or by stereotactic injection (Chen et al., 1994). The pharmaceutical preparation of a gene therapy vector can include an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

2. Vectors

Vectors are tools used to shuttle DNA between host cells or as a means to express a nucleotide sequence. Some vectors function only in prokaryotes, while others function in both prokaryotes and eukaryotes, enabling large-scale DNA preparation from prokaryotes for expression in eukaryotes. Inserting the DNA of interest, such as a msx1 nucleotide sequence or a fragment, is accomplished by ligation techniques and/or mating protocols well known to the skilled artisan. Such DNA is inserted such that its integration does not disrupt any functional components of the vector. Introduced DNA is operably-linked to the vector elements that govern transcription and translation in vectors that express the introduced DNA.

Vectors can be divided into two general classes: Cloning vectors are replicating plasmids or phage with regions that are non-essential for propagation in an appropriate host cell and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA. In expression vectors, the introduced DNA is operably-linked to elements such as promoters that signal to the host cell to transcribe the inserted DNA. Some promoters are exceptionally useful, such as inducible promoters that control gene transcription in response to specific factors. Operably-linking msx1 or anti-sense constructs to an inducible promoter can control the expression of msx1 or fragments or anti-sense constructs. Examples of classic inducible promoters include those that are responsive to a-interferon, heat-shock, heavy metal ions, and steroids such as glucocorticoids (Kaufman, 1990) and tetracycline. Other desirable inducible promoters include those that are not endogenous to the cells in which the construct is being introduced, but, however, are responsive in those cells when the induction agent is exogenously supplied.

Vectors have many different manifestations. A “plasmid” is a circular double stranded DNA molecule into which additional DNA segments can be introduced. Viral vectors can accept additional DNA segments into the viral genome. Certain vectors are capable of autonomous replication in a host cell (e.g., bacterial vectors having a bacterial origin of replication and episomal mammalian vectors). Other vectors (e.g., non-episomal mammalian vectors) are integrated into the genome of a host cell upon introduction into the host cell, and are replicated along with the host genome. In general, useful expression vectors are often plasmids. However, other forms of expression vectors, such as viral vectors (e.g., replication defective retroviruses, adenoviruses and adeno-associated viruses) are contemplated. Such vectors can be extremely useful in gene therapy applications.

Recombinant expression vectors that comprise msx1 (or fragments) regulate msx1 transcription by exploiting one or more host cell-responsive (or that can be manipulated in vitro) regulatory sequences that is operably-linked to msx1. “Operably-linked” indicates that a nucleotide sequence of interest is linked to regulatory sequences such that expression of the nucleotide sequence is achieved.

Vectors can be introduced in a variety of organisms and/or cells (Table D). Alternatively, the vectors can be transcribed and translated in vitro, for example using T7 promoter regulatory sequences and T7 polymerase.

TABLE D
Examples of hosts for cloning or expression

| Organisms | Examples | Sources and References* |
|---|---|---|
| Prokaryotes | E. coli | |
| Enterobacteriaceae | K 12 strain MM294 | ATCC 31,446 |
| | X1776 | ATCC 31,537 |
| | W3110 | ATCC 27,325 |
| | K5 772 | ATCC 53,635 |
| | Enterobacter | |
| | Erwinia | |
| | Klebsiella | |
| | Proteus | |
| | Salmonella | |
| | (S. tyhpimurium) | |
| | Serratia | |
| | (S. marcescans) | |
| | Shigella | |
| | Bacilli | |
| | (B. subtilis and | |
| | B. licheniformis) | |
| | Pseudomonas | |
| | (P. aeruginosa) | |
| | Streptomyces | |
| Eukaryotes | Saccharomyces | |
| | cerevisiae | |
| Yeasts | Schizosaccharomyces | |
| | pombe | |
| | Kluyveromyces | (Fleer et al., 1991) |
| | K. lactis MW98-8C, | (de Louvencourt et al., |
| | CBS683, CBS4574 | 1983) |
| | K. fragilis | ATCC 12,424 |
| | K. bulgaricus | ATCC 16,045 |
| | K. wickeramii | ATCC 24,178 |
| | K. waltii | ATCC 56,500 |
| | K. drosophilarum | ATCC 36,906 |
| | K. thermotolerans | |
| | K. marxianus; | (EPO 402226, 1990) |
| | yarrowia | |
| | Pichia pastoris | (Sreekrishna et al., 1988) |
| | Candida | |
| | Trichoderma reesia | |
| | Neurospora crassa | (Case et al., 1979) |
| | Torulopsis | |
| | Rhodotorula | |
| | Schwanniomyces | |
| | (S. occidentalis) | |
| Filamentous Fungi | Neurospora | |
| | Penicillium | |
| | Tolypocladium | (WO 91/00357, 1991) |
| | Aspergillus | (Kelly and Hynes, 1985; |
| | (A. nidulans and | Tilburn et al., 1983; |
| | A. niger) | Yelton et al., 1984) |
| Invertebrate cells | Drosophila S2 | |
| | Spodoptera Sf9 | |
| Vertebrate cells | Chinese | |
| | Hamster Ovary | |
| | (CHO) | |
| | simian COS | ATCC CRL 1651 |
| | COS-7 | |
| | HEK 293 | |

*Unreferenced cells are generally available from American Type Culture Collection (Manassas, VA).

Vector choice is dictated by the organism or cells being used and the desired fate of the vector. Vectors may replicate once in the target cells, or may be “suicide” vectors. In general, vectors comprise signal sequences, origins of replication, marker genes, enhancer elements, promoters, and transcription termination sequences. The choice of these elements depends on the organisms in which the vector will be used and are easily determined. Some of these elements may be conditional, such as an inducible or conditional promoter that is turned “on” when conditions are appropriate. Examples of inducible promoters include those that are tissue-specific, which relegate expression to certain cell types, steroid-responsive, or heat-shock reactive. Some bacterial repression systems, such as the lac operon, have been exploited in mammalian cells and transgenic animals (Fieck et al., 1992; Wyborski et al., 1996; Wyborski and Short, 1991). Vectors often use a selectable marker to facilitate identifying those cells that have incorporated the vector. Many selectable markers are well known in the art for the use with prokaryotes, usually antibiotic-resistance genes or the use of autotrophy and auxotrophy mutants.

If msx1 expression is not desired, using antisense and sense msx1 oligonucleotides can prevent msx1 polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target msx1 mRNA (sense) or msx1 DNA (antisense) sequences. According to the present invention, antisense or sense oligonucleotides comprise a fragment of the msx1 DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Among others, (Stein and Cohen, 1988; van der Krol et al., 1988) describe methods to derive antisense or a sense oligonucleotides from a given cDNA sequence.

Modifications of antisense and sense oligonucleotides can augment their effectiveness. Modified sugar-phosphodiester bonds or other sugar linkages (WO 91/06629, 1991), increase in vivo stability by conferring resistance to endogenous nucleases without disrupting binding specificity to target sequences. Other modifications can increase the affinities of the oligonucleotides for their targets, such as covalently linked organic moieties (WO 90/10448, 1990) or poly-(L)-lysine. Other attachments modify binding specificities of the oligonucleotides for their targets, including metal complexes or intercalating (e.g. ellipticine) and alkylating agents.

To introduce antisense or sense oligonucleotides into target cells (cells containing the target nucleic acid sequence), any gene transfer method may be used and these methods are well known to those of skill in the art. Examples of gene transfer methods include (1) biological, such as gene transfer vectors like Epstein-Barr virus or conjugating the exogenous DNA to a ligand-binding molecule (WO 91/04753, 1991), (2) physical, such as electroporation, and (3) chemical, such as CaPO₄ precipitation and oligonucleotide-lipid complexes (WO 90/10448, 1990).

The terms “host cell” and “recombinant host cell” are used interchangeably. Such terms refer not only to a particular subject cell but also to the progeny or potential progeny of such a cell. Because certain modifications may occur in succeeding generations due to either mutation or environmental influences, such progeny may not, in fact, be identical to the parent cell, but are still included within the scope of the term.

Methods of eukaryotic cell transfection and prokaryotic cell transformation are well known in the art. The choice of host cell will dictate the preferred technique for introducing the nucleic acid of interest. Table E, which is not meant to be limiting, summarizes many of the known techniques in the art. Introduction of nucleic acids into an organism may also be done with ex vivo techniques that use an in vitro method of transfection, as well as established genetic techniques, if any, for that particular organism.

TABLE E
Methods to introduce nucleic acid into cells

| Cells | Methods | References | Notes |
|---|---|---|---|
| Prokaryotes | Calcium chloride | (Cohen et al., 1972; | |
| (bacteria) | | Hanahan, 1983; Mandel and | |
| | | Higa, 1970) | |
| | Electroporation | (Shigekawa and Dower, | |
| | | 1988) | |
| Eukaryotes | Calcium phosphate | N-(2- | Cells may be |
| Mammalian cells | transfection | Hydroxyethyl)piperazine-N′- | “shocked” with |
| | | (2-ethanesulfonic acid | glycerol or |
| | | (HEPES) buffered saline | dimethylsulfoxide |
| | | solution (Chen and | (DMSO) to increase |
| | | Okayama, 1988; Graham | transfection |
| | | and van der Eb, 1973; | efficiency (Ausubel |
| | | Wigler et al., 1978) | et al., 1987). |
| | | BES (N,N-bis(2- | |
| | | hydroxyethyl)-2- | |
| | | aminoethanesulfonic acid) | |
| | | buffered solution (Ishiura et | |
| | | al., 1982) | |
| | Diethylaminoethyl | (Fujita et al., 1986; Lopata et | Most useful for |
| | (DEAE)-Dextran | al., 1984; Selden et al., 1986) | transient, but not |
| | transfection | | stable, transfections. |
| | | | Chloroquine can be |
| | | | used to increase |
| | | | efficiency. |
| | Electroporation | (Neumann et al., 1982; | Especially useful for |
| | | Potter, 1988; Potter et al., | hard-to-transfect |
| | | 1984; Wong and Neumann, | lymphocytes. |
| | | 1982) | |
| | Cationic lipid | (Elroy-Stein and Moss, | Applicable to both |
| | reagent | 1990; Felgner et al., 1987; | in vivo and in vitro |
| | transfection | Rose et al., 1991; Whitt et | transfection. |
| | | al., 1990) | |
| | Retroviral | Production exemplified by | Lengthy process, |
| | | (Cepko et al., 1984; Miller | many packaging |
| | | and Buttimore, 1986; Pear et | lines available at |
| | | al., 1993) | ATCC. Applicable |
| | | Infection in vitro and in vivo: | to both in vivo and |
| | | (Austin and Cepko, 1990; | in vitro transfection. |
| | | Bodine et al., 1991; Fekete | |
| | | and Cepko, 1993; Lemischka | |
| | | et al., 1986; Turner et al., | |
| | | 1990; Williams et al., 1984) | |
| | Polybrene | (Chaney et al., 1986; Kawai | |
| | | and Nishizawa, 1984) | |
| | Microinjection | (Capecchi, 1980) | Can be used to |
| | | | establish cell lines |
| | | | carrying integrated |
| | | | copies of msx 1 |
| | | | DNA sequences. |
| | | | Applicable to both |
| | | | in vitro and in vivo. |
| | Protoplast fusion | (Rassoulzadegan et al., 1982; | |
| | | Sandri-Goldin et al., 1981; | |
| | | Schaffner, 1980) | |
| Insect cells | Baculovirus | (Luckow, 1991; Miller, | Useful for in vitro |
| (in vitro) | systems | 1988; O'Reilly et al., 1992) | production of |
| | | | proteins with |
| | | | eukaryotic |
| | | | modifications. |
| Yeast | Electroporation | (Becker and Guarente, 1991) | |
| | Lithium acetate | (Gietz et al., 1998; Ito et al., | |
| | | 1983) | |
| | Spheroplast fusion | (Beggs, 1978; Hinnen et al., | Laborious, can |
| | | 1978) | produce aneuploids. |
| Plant cells | Agrobacterium | (Bechtold and Pelletier, | |
| (general | transformation | 1998; Escudero and Hohn, | |
| reference: | | 1997; Hansen and Chilton, | |
| (Hansen and | | 1999; Touraev and al., 1997) | |
| Wright, | Biolistics | (Finer et al., 1999; Hansen | |
| 1999)) | (microprojectiles) | and Chilton, 1999; Shillito, | |
| | | 1999) | |
| | Electroporation | (Fromm et al., 1985; Ou-Lee | |
| | (protoplasts) | et al., 1986; Rhodes et al., | |
| | | 1988; Saunders et al., 1989) | |
| | | May be combined with | |
| | | liposomes (Trick and al., | |
| | | 1997) | |
| | Polyethylene | (Shillito, 1999) | |
| | glycol (PEG) | | |
| | treatment | | |
| | Liposomes | May be combined with | |
| | | electroporation (Trick and | |
| | | al., 1997) | |
| | in planta | (Leduc and al., 1996; Zhou | |
| | microinjection | and al., 1983) | |
| | Seed imbibition | (Trick and al., 1997) | |
| | Laser beam | (Hoffman, 1996) | |
| | Silicon carbide | (Thompson and al., 1995) | |
| | whiskers | | |

Vectors often use a selectable marker to facilitate identifying those cells that have incorporated the vector, especially in vitro. Many selectable markers are well known in the art for prokaryotic selection, usually antibiotic-resistance genes or the use of autotrophy and auxotrophy mutants. Table F lists common selectable markers for mammalian cell transfection.

TABLE F
Useful selectable markers for eukaryote cell transfection

| Selectable Marker | Selection | Action | Reference |
|---|---|---|---|
| Adenosine deaminase | Media includes 9-β-D- | Conversion of Xyl-A to | (Kaufman |
| (ADA) | xylofuranosyl adenine | Xyl-ATP, which | et al., 1986) |
| | (Xyl-A) | incorporates into | |
| | | nucleic acids, killing | |
| | | cells. ADA detoxifies | |
| Dihydrofolate | Methotrexate (MTX) | MTX competitive | (Simonsen |
| reductase (DHFR) | and dialyzed serum | inhibitor of DHFR. In | and |
| | (purine-free media) | absence of exogenous | Levinson, |
| | | purines, cells require | 1983) |
| | | DHFR, a necessary | |
| | | enzyme in purine | |
| | | biosynthesis. | |
| Aminoglycoside | G418 | G418, an | (Southern |
| phosphotransferase | | aminoglycoside | and Berg, |
| (“APH”, “neo”, | | detoxified by APH, | 1982) |
| “G418”) | | interferes with | |
| | | ribosomal function and | |
| | | consequently, | |
| | | translation. | |
| Hygromycin-B- | hygromycin-B | Hygromycin-B, an | (Palmer et |
| phosphotransferase | | aminocyclitol detoxified | al., 1987) |
| (HPH) | | by HPH, disrupts | |
| | | protein translocation | |
| | | and promotes | |
| | | mistranslation. | |
| Thymidine kinase | Forward selection | Forward: Aminopterin | (Littlefield, |
| (TK) | (TK+): Media (HAT) | forces cells to synthesze | 1964) |
| | incorporates | dTTP from thymidine, a | |
| | aminopterin. | pathway requiring TK. | |
| | Reverse selection | Reverse: TK | |
| | (TK−): | phosphorylates BrdU, | |
| | Media incorporates | which incorporates into | |
| | 5-bromodeoxyuridine | nucleic acids, killing | |
| | (BrdU). | cells. | |

3. Production of msx1 In Vitro

A host cell, such as a prokaryotic or eukaryotic host cell, can be used to produce msx1. Host cells that are useful for in vitro production of msx1 or msx1 fusion polypeptides, into which a recombinant expression vector encoding msx1 has been introduced, include as nonlimiting examples, E. coli, COS7, and Drosophila S2. Preferably, such cells do not modify the produced polypeptide in such as way that when introduced into a subject, such as a human, an immune response is evoked. For example, certain sugar post-translational modifications may provoke such a response. Preferably, such cells produce active polypeptides. The cells are cultured in a suitable medium, such that msx1 or the desired polypeptide is produced. If necessary msx1 is isolated from the medium or the host cell. Likewise, Fgfs may be similarly produced, using the appropriate corresponding polynucleotides.

D. Cell Culture

Suitable medium and conditions for generating primary cultures are well known in the art and vary depending on cell type, can be empirically determined. For example, skeletal muscle, bone, neurons, skin, liver, and embryonic stem cells are all grown in media differing in their specific contents. Furthermore, media for one cell type may differ significantly from lab to lab and institution to institution. To keep cells dividing, serum, such as fetal calf serum, is added to the medium in relatively large quantities, 5%-30% by volume, again depending on cell or tissue type. Specific purified growth factors or cocktails of multiple growth factors can also be added or are sometimes substituted for serum. When differentiation is desired and not proliferation, serum with its mitogens is generally limited to about 0-2% by volume. Specific factors or hormones that promote differentiation and/or promote cell cycle arrest can also be used.

Physiologic oxygen and subatmospheric oxygen conditions can be used at any time during the growth and differentiation of cells in culture, as a critical adjunct to selection of specific cell phenotypes, growth and proliferation of specific cell types, or differentiation of specific cell types. In general, physiologic or low oxygen-level culturing is accompanied by methods that limit acidosis of the cultures, such as addition of strong buffer to medium (such as HEPES), and frequent medium changes and changes in CO₂ concentration.

In addition to oxygen, the other gases for culture typically are about 5% carbon dioxide and the remainder is nitrogen, but optionally may contain varying amounts of nitric oxide (starting as low as 3 ppm), carbon monoxide and other gases, both inert and biologically active. Carbon dioxide concentrations typically range around 5%, but may vary between 2-10%. Both nitric oxide and carbon monoxide, when necessary, are typically administered in very small amounts (i.e. in the ppm range), determined empirically or from the literature.

The medium can be supplemented with a variety of growth factors, cytokines, serum, etc. Examples of suitable growth factors are basic fibroblast growth factor (bFGF), vascular endothelial growth factor (VEGF), epidermal growth factor (EGF), transforming growth factors (TGFα and TGFβ), platelet derived growth factors (PDGFs), hepatocyte growth factor (HGF), insulin-like growth factor (IGF), insulin, erythropoietin (EPO), and colony stimulating factor (CSF). Examples of suitable hormone medium additives are estrogen, progesterone, testosterone or glucocorticoids such as dexamethasone. Examples of cytokine medium additives are interferons, interleukins, or tumor necrosis factor-α (TNFα). One skilled in the art will test additives and culture components in different culture conditions, as these may alter cell response, active lifetime of additives or other features affecting their bioactivity. In addition, the surface on which the cells are grown can be plated with a variety of substrates that contribute to survival, growth and/or differentiation of the cells. These substrates include but are not limited to laminin, EHS-matrix, collagen, poly-L-lysine, poly-D-lysine, polyornithine and fibronectin. In some instances, when 3-dimensional cultures are desired, extracellular matrix gels may be used, such as collagen, EHS-matrix, or gelatin. Cells may be grown on top of such matrices, or may be cast within the gels themselves.

E. Dedifferentiating Cells

1. Myotubes In Vitro

Myotubes, isolated from a subject, preferably a human, or generated from murine myoblast cell lines (see examples) are cultured in vitro in sutiable media.

A skilled artisan will know how to vary the conditions set forth to achieve dedifferentiation. A skilled artisan will know how to vary the conditions set forth to achieve dedifferentiation. The following description is set forth as an illustrative example.

To induce dedifferentiation of myotubes in culture, RE is added to differentiation medium (see Examples) at a suitable time after plating the cells at low density onto an appropriate substrate (e.g. tissue culture plastic, gelatin, fibronectin, laminin, collagen, EHS-matrix, etc.-coated surfaces). Medium and extract are preferably changed daily. To identify morphologic dedifferentiation, individual cells are photographed on day 0, before the addition of extract, and every 24 hrs after the addition of extract for up to 10 days or longer.

2. Differentiated Cells In Vitro

Cells isolated from a subject, preferably a human, or generated from cell lines are cultured ill vitro in sutiable media.

A skilled artisan will know how to vary the conditions set forth to achieve dedifferentiation. The following description is set forth as an illustrative example.

To induce dedifferentiation of cells in culture, RE is added to differentiation medium (see Examples) at a suitable time after plating the cells at low density onto an appropriate substrate (e.g. tissue culture plastic, gelatin, fibronectin, laminin, collagen, EHS-matrix, etc. -coated surfaces or in suspension). Medium and extract are preferably changed daily. To identify morphologic dedifferentiation, individual cells are photographed on day 0, before the addition of extract, and every 24 hrs after the addition of extract for up to 10 days or longer.

3. Cells In Vivo

Cells, preferably at a site of injury, are contacted with RE. RE may be formulated within a pharmaceutical composition to ensure delivery.

F. Pharmaceutical Compositions

The compositions of the invention (RDF components) and derivatives, fragments, analogs and homologues thereof, can be incorporated into pharmaceutical compositions. Such compositions typically comprise the nucleic acid molecule, protein, or antibody and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (Gennaro, 2000). Preferred examples of such carriers or diluents include, but are not limited to, water, saline, finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

The pharmaceutical compositions for the administration of the active compounds, such as those of RDF, may conveniently be presented in dosage unit form and may be prepared by any of the methods well known in the art of pharmacy. All methods include the step of bringing the active compound into association with the carrier that constitutes one or more accessory ingredients. In general, the pharmaceutical compositions are prepared by uniformly and intimately bringing the active compound into association with a liquid carrier or a finely divided solid carrier or both, and then, if necessary, shaping the product into the desired formulation. In the pharmaceutical composition the active compound is included in an amount sufficient to produce the desired effect upon the process or condition of diseases.

1. General Considerations

A pharmaceutical composition of the invention is formulated to be compatible with its intended route of administration, including intravenous, intradermal, subcutaneous, oral (e.g., inhalation), transdermal (i.e., topical), transmucosal, and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

2. Injectable Formulations

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents, for example, parabens, chlorobutanol, phenol, ascorbic acid, and thimerosal, can contain microorganism contamination. Isotonic agents, for example, sugars, polyalcohols such as manitol, sorbitol, and sodium chloride can be included in the composition. Compositions that can delay absorption include agents such as aluminum monostearate and gelatin.

Sterile injectable solutions can be prepared by incorporating the active compound or composition in the required amount in an appropriate solvent with one or a combination of ingredients as required, followed by sterilization. Generally, dispersions are prepared by incorporating the active compound into a sterile vehicle that contains a basic dispersion medium, and the other required ingredients as discussed. Sterile powders for the preparation of sterile injectable solutions, methods of preparation include vacuum drying and freeze-drying that yield a powder containing the active ingredient and any desired ingredient from a sterile solutions.

3. Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

4. Compositions for Inhalation

For administration by inhalation, the compounds are delivered as an aerosol spray from a nebulizer or a pressurized container that contains a suitable propellant, e.g., a gas such as carbon dioxide.

5. Systemic Administration, Including Patches

Systemic administration can also be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams.

The compounds can also be prepared in the form of suppositories (e.g., with bases such as cocoa butter and other glycerides) or retention enemas for rectal delivery.

6. Carriers

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc. (Lake Elsinore, Calif.), or prepared by one of skill in the art. Liposomal suspensions can also be used as pharmaceutically acceptable carriers. These can be prepared according to methods known to those skilled in the art, such as in (Eppstein et al., U.S. Pat. No. 4,522,811, 1985).

7. Unit Dosage

Oral formulations or parenteral compositions in unit dosage form can be created to facilitate administration and dosage uniformity. Unit dosage form refers to physically discrete units suited as single dosages for the subject to be treated, containing a therapeutically effective quantity of active compound in association with the required pharmaceutical carrier. The specification for the unit dosage forms of the invention are dictated by, and directly dependent on, the unique characteristics of the active compound and the particular desired therapeutic effect, and the inherent limitations of compounding the active compound.

8. Dosage

The pharmaceutical composition and method of the present invention may further comprise other therapeutically active compounds as noted herein that are usually applied in the treatment of wounds or other associated pathological conditions.

In the treatment of conditions which require tissue regeneration or cellular dedifferention, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. In addition, the site of delivery will also impact dosage and frequency.

Combined therapy to engender tissue regeneration is illustrated by the combination of the compositions of this invention and other compounds that are known for such utilities.

9. Kits for Pharmaceutical Compositions

The pharmaceutical compositions can be included in a kit, container, pack, or dispenser together with instructions for administration. When the invention is supplied as a kit, the different components of the composition may be packaged in separate containers and admixed immediately before use. Such packaging of the components separately may permit long-term storage without losing the active components' functions.

(a) Containers or Vessels

The reagents included in the kits can be supplied in containers of any sort such that the life of the different components are preserved, and are not adsorbed or altered by the materials of the container. For example, sealed glass ampoules may contain lyophilized RE, RDF or buffer that have been packaged under a neutral, non-reacting gas, such as nitrogen. Ampoules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampules, and envelopes, that may consist of foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

(b) Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, etc. Detailed instructions may not be physically associated with the kit; instead, a user may be directed to an internet web site specified by the manufacturer or distributor of the kit, or supplied as electronic mail.

H. Delivery Methods (Needs to be Modified and Adapted for this Application)

1 Interstitial Delivery

The composition of the invention may be delivered to the interstitial space of tissues of the animal body, including those of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheatling muscle cells or in the lacunae of bone. It is similarly the space occupied by the plasma of the circulation and the lymph fluid of the lymphatic channels. They may be conveniently delivered by injection into the tissues comprising these cells. They are preferably delivered to sites of injury, preferably to live cells and extracellular matrices directly adjacent to dead and dying tissue.

Any apparatus known to the skilled artisan in the medical arts may be used to deliver the compositions of the invention to the site of injury interstitially. These include, but are not limited to, syringes, stents and catheters.

2. Systemic Delivery

In the case of damaged tissue throughout a subject, or in the blood vessels (or lymph system) themselves, then delivery into the circulation system may be desired. Any apparatus known to the skilled artisan in the medical arts may be used to deliver the compositions of the invention to the circulation system. These include, but are not limited to, syringes, stents and catheters. One convenient method is delivery via intravenous drip. Another approach would comprise implants, such as transdermal patches, that deliver the comopositions of the invention over prolonged periods of time. Such implants may or may not be absorbed by the subject over time.

3. Surgical Delivery

During surgical procedures, the methods and compositions of the invention can be advantageously used to simplify the surgery of interest, such as reducing the amount of intervention, as well as to repair the damage wrought by the surgical procedure. The compositions of the invention may be delivered in a way that is appropriate for the surgery, including by bathing the area under surgery,implantable drug delivery systems, and matrices (absorbed by the body over time) impregnated with the compositions of the invention.

4. Superficial Delivery

In the case of injuries to, or damaged tissues on, the exterior surfaces of a subject, direct application of the compositions of the invention is preferred. For example, a gauze impregnated with RDF components, may be directly applied to the site of damage, and may be held in place, such as by a bandage or other wrapping. Alternatively, the compositions of the invention may be applied in salves, creams, or other pharmaceutical compositions known in the art meant for topical application.

### EXAMPLES

The following examples are included to demonstrate preferred embodiments of the present invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing form the spirit and scope of the invention.

1.1 Animals/Tissue Collection

Adult newts, Notophthalamus viridescens, from Charles Sullivan & Co. (Tennessee), were maintained in a humidified room at 24° C. and fed Tubifex worms 2-3×/wk. Operations were performed on animals anesthetized with 0.1% tricaine for approximately 2-3 minutes. Regenerating limb tissue was collected as follows. Forelimbs were amputated by cutting just proximal to the elbow and soft tissue was pushed up the humorus to expose the bone. The bone and soft tissue were trimmed to produce a flat amputation surface. The newts were placed in 0.5% sulfamerazine solution overnight and then back into a normal water environment. Early regenerating tissue (days 1, 3, and 5 postamputation) was collected by reamputating the limb 0.5-1.0 mm proximal to the wound epithelum and removing any residual bone. Nonregenerating limb tissue was collected from limbs that had not been previously amputated. Tissue was extracted 2-3 mm proximal to the forelimb elbow and all bones were removed. Immediately after collection, all tissues were flash frozen in liquid nitrogen and stored at −80° C.

1.2 Preparation of Protein Extracts

Tissues were thawed and all subsequent manipulations were performed at 4° C. or on ice. Six grams of early regenerating tissue from days 1, 3, and 5 (2 g each) or 6 g of nonregenerating tissue were placed separately into 10 ml of Dulbecco's Modified Eagle's Medium (DMEM; GIBCO-BRL No. 11995-065; Carlsbad, Calif.) containing protease inhibitors (2 μg/ml leupeptin, 2 μg/ml A-protinin, and 1 mM phenylmethylsulfonyl fluoride (PMSF)). The tissues were ground with an electronic tissue homogenizer for 1-2 minutes, hand homogenized for 10-15 minutes, and sonicated for 30 seconds. Cell debris was removed in two centrifugation steps. The homogenate was first spun at 2000 g for 25 minutes and then the supematant was spun again at 100,000 g for 60 minutes. The nonsoluble lipid layer was aspirated and the remaining supernatant filter sterilized through a 0.45 μm filter. The protein content was assayed with a BCA protein assay kit (Pierce; Rockford, Ill.) and stored in 0.5 ml aliquots at −80° C.

1.3 Cell Culture

Newt A1 limb cells were obtained as a gift from Jeremy Brockes (Department of Biochemistry and Molecular Biology, University College London, London, United Kingdom). Mouse C2C12 myoblast cell line was purchased from ATCC. Newt A1 cells were passaged, myogenesis induced, and myotubes isolated and plated at low density (Ferretti and Brockes, 1988; Lo et al., 1993). Newt A1 cells were grown at 24° C. in 2% CO₂. The culture medium was adjusted to the axolotl plasma osmolality of 225 Osm (Ferretti and Brockes, 1988) using an Osmette A Automated Osmometer (Precision Scientific, Inc.; Winchester, Va.). Culture medium contained Minimal Essential Medium (MEM) with Eagle's salt, 10% fetal bovine serum (FBS, Clontech No. 8630-1), 100 U/ml penicillin, 100 μg/ml streptomycin, 0.28 IU/ml bovine pancreas insulin, 2 mM glutamine, and distilled water.

To induce myotube formation in newt A1 cells, mononucleated cells were grown to confluency and the above medium was replaced with medium containing 0.5% FBS (Differentiation Medium; DM) for 4-6 days. These myotubes were isolated from remaining mononucleated cells by gentle trypsinization (0.05% trypsin) and sequentially sieved through 100 μm and 35 μm nylon meshes. Larger debris and clumped cells were retained on the first sieve, most myotubes were retained on the second sieve, and most mononucleated cells passed through both sieves. Myotubes were gently washed off the 35 μm sieve and plated at either 1-2 myotubes/hpf or <0.25 myotube/hpf onto 35 mm plates precoated with 0.75% gelatin.

C2C12 cells were passaged and myogenesis induced as previously described (Guo et al., 1995). C2C12 myotubes were isolated and plated at low density after gentle trypsinization and sieving through 100 μm mesh. Myotubes were retained on this sieve while mononucleated cells passed through. Myotubes were washed off the sieve and plated at either 1-2 myotubes/hpf or <0.25 myotubes/hpf onto 35 mm plates precoated with 0.75% gelatin.

To induce dedifferentiation of myotubes, 0.1-0.3 mg/ml of RNLE was added to DM 24 hrs after plating at low density (<0.25 myotubes/hpf) in 35 mm gelatin coated plates. Medium and extract were changed daily. To identify morphologic dedifferentiation, individual myotubes were photographed on day 0, before the addition of extract, and every 24 hrs after the addition of extract for up to 10 days. To test for myotube downregulation of muscle specific markers as well as reentry into the phase of the cell cycle, the cells were plated at slightly higher density (1-2 cells/hpf) with medium and extract changed daily. The cells were stained as described below on day four. Cells cultured in DM alone or in DM with nonRNLE were used as negative controls.

1.4 Immunofluorescence Microscopy

Cells plated at low density in 35 mm plates were washed three times with phosphate buffered saline (PBS) before fixation and immunostaining. Unless otherwise specified, all manipulations were at room temperature, all dilutions of antibodies were prepared in 2% normial goat serum (NGS)/0.1% nonylphenoxy polyethoxy ethanol (NP-40) in PBS, and incubations were followed by washes with 0.1% NP-40 in PBS. Cells were fixed in cold methanol at −20° C. for 10 minutes, rehydrated with PBS, and blocked with 10% NGS for 15 minutes.

TABLE I
Primary antibodies

| Antigen | Antibody type | Dilution | Source |
|---|---|---|---|
| troponin T | mAb | 1:50 | Sigma #T6277 |
| myogenin | mAb (F5D clone) | 1:50 | Pharmingen |
| | | | #65121A |
| myoD | NCL-myoD1 | 1:10 | Vector |
| | mouse mAb | | Laboratories, Inc. |
| p21 | WAF1 rabbit | 1:100 | Oncogene Research |
| | polyclonal | | Products |
| | antibody | | |

Primary antibodies were incubated for 1 hour at 37° C. After three washes, cells were incubated 45 minutes at 37° C. with secondary antibody. For troponin T, a goat anti-mouse IgG conjugated to Alexa 594 (1:100 dilution, Molecular Probes; Eugene, Oreg.) was used, while myogenin and myoD required biotin-xx goat anti-mouse IgG (1:200 dilution, Molecular Probes), followed by 45 minute incubation with streptavidin Alexa 594 (1: 100 dilution, Molecular Probes). No cross-reactivity of the secondary antibodies was observed in control experiments in which primary antibodies were omitted.

In some experiments, cells were counterstained with bromodeoxyuridine (BrdU) for 12 hours, using a 5-bromo-2′-deoxy-uridine labeling and detection kit I according to manufacturer's instructions (Boehringer Mannheim (Roche); Indianapolis, Ind.). Cells were examined microscopically and photographed using a Zeiss Axiovert 100 equipped with a mounted camera and fluorescent source.

For cells transformed with msx1 (see below), inducing C2C12 cells, Fwd clones, and the Rev clone to differentiate in the presence of DM-doxycycline (DM-dox) produced myotubes. Myotubes were then gently trypsinized and replated at low density in DM-dox. The following day, the medium was replaced with growth medium (GM) to induce msx1 expression in the presence of growth factors. Cells were analyzed for myoD, myogenin and p21 expression by immunofluorescence on day 0 (before induction) through day 3 (postinduction). Secondary antibodies were used at 1:200 dilution and included a biotinylated goat anti-mouse IgG antibody (B-2763, Molecular Probes) and an Alexa 488-conjugated goat anti-rabbit IgG antibody (A-11034, Molecular Probes). Myotubes were rinsed three times with Dulbecco's phosphate buffered saline (DPBS), treated with Zamboni's fixative for 10 minutes, washed once with DPBS, and permeabilized with 0.2% Triton-X-100 in DPBS for 20 minutes. The myotubes were blocked with 5% skim milk in DPBS for 1 hour and then exposed to two primary antibodies (one was a mouse monoclonal, the other a rabbit polyclonal overnight at 4° C.) The cells were washed three times with DPBS and then treated with two secondary antibodies (a goat anti-rabbit IgG conjugated to Alexa 488 (Molecular Probes) and a goat anti-mouse IgG conjugated to biotin) for 45 minutes at 37° C. Myotubes were washed three times with DPBS and then exposed to 1 μg/ml streptavidin-Alexa 594 (S-11227, Molecular Probes) for 45 minutes at 37° C. The cells were washed three times with DPBS and observed with a Zeiss Axiovert 100 inverted microscope using FITC and Texas Red filters.

1.5 Characterization of the Newt Regeneration Lysate Activity

C2C12 myotubes were plated at low density in DM as described above. Regeneration extract was treated in one of three ways: (1) boiled for 5 minutes; (2) digested with 1% trypsin for 30 minutes at 37° C.; or (3) taken through several freeze/thaw cycles. In three separate experiments, the treated extracts were applied to cultured myotubes at a concentration of 0.3 mg/ml with media and extract changed daily. Immediately after the extract was digested with 1% trypsin, the trypsin was inactivated by dilution in DM in which the cells were cultured. In the freeze/thaw experiments, extract activity was tested after both 2 and 3 freeze/thaw cycles. The effect of the pretreated extracts on myotube S phase reentry was assessed after 4 days of treatment by performing BrdU incorporation assays. The results were compared to BrdU incorporation in myotubes cultured in DM containing RNLE (positive control) and myotubes cultured in DM alone or DM containing nonRNLE (negative controls).

1.6 Construction of msx1 in a Retroviral Vector

A 1.2 kb DNA fragment containing the entire coding region of the mouse msx1 gene was excised from the plasmid phox7XS using SacI and XbaI, blunt-ended with dNTPs and Klenow fragment, and ligated into the LINX retroviral vector at the blunted ClaI site. Clones containing the msx1 gene in both the forward (LINX-msx1-fwd) and reverse (LINX-msx1-rev) orientations were identified and used for the transduction studies.

1.7 Transduction of C2C12 Cells and Selection of Clones Harboring Inducible msx1

Phoenix-Ampho cells (ATCC No. SD3443) were grown to 70-80% confluency in growth medium (GM) containing 10% tetracycline-tested FBS, 2 mM glutamine, 100 μg/ml penicillin, 100 units/ml streptomycin, and DMEM. Cells were transfected for 10 hours. Medium was replaced and cells were grown an additional 48 hours. The retroviral-containing conditioned medium was then harvested and live cells were removed by centrifugation at 500 g.

C2C12 cells were grown to 20% confluency in GM containing 20% tetracycline-tested FBS, 4 mM glutamine, 2 μg/ml doxycycline, and DMEM. C2C12 cells were infected with the LINX-msx1-fwd or LINX-msx1-rev recombinant retroviruses in T25 tissue culture flasks by replacing GM with retroviral-containing medium comprised of 1 ml retroviral conditioned medium, 2 ml GM, and 4 μg/ml Polybrene: Cells were incubated at 37° C./5% CO₂ for 12-18 hours, and the medium was replaced with fresh GM. The cells were incubated an additional 48 hours and then switched to a 37° C./10% CO₂ incubator. Cells were split just before they reached confluency and selection in G418 (750 μg/ml) was initiated. Selection continued for 6 days and then the cells were split into 100 mm tissue culture plates at a density of 50 cells/plate. Selection was continued for an additional 8 days. Individual cell colonies were isolated using cloning cylinders, and these clones were expanded in GM-G418. Clones were tested for inducible msx1 expression by Northern analysis of total RNA and inhibition of myocyte differentiation in reduced growth factor medium.

1.8 Morphological Dedifferentiation Assays

Myotubes were prepared as described above, gently trypsinized with 0.25% trypsin/1 mM EDTA and replated in DM-dox at a density of 2-4 myotubes/mm² on gridded 35 mm gelatinized plates. The following day residual mononucleated cells were destroyed by lethal injection of water and/or needle ablation using an Eppendorf microinjection system (Westbury, N.Y.). The myotubes were then induced to express msx1 in the presence of growth factors by replacing the culture medium with GM (minus doxycycline). The cells were observed and photographed every 12-24 hours for up to seven days.

1.9 Transdetermination and Pluripotency Assays for Dedifferentiated Cells

Msx1 expression was induced in Fwd clones for five days in the absence of doxycycline (dox) and then suppressed an additional five days in the presence of 2 μg/ml doxycycline. Control msx1-rev and C2C12 cells were similarly treated. In addition, two clonal populations of cells derived from a dedifferentiated Fwd-2 myotube were obtained by plating at limiting dilution in 96-well plates. The above cells were used in the following assays for transdetermination and pluripotency.

Chondorgenic Potential

Chondrogenic potential was assessed in the presence of 2 μg/ml doxycycline according to published protocols (Dennis et al., 1999; Mackay et al., 1998). The cell pellets were treated with O.C.T. compound (Tissue-Tek), frozen in a dry ice/ethanol bath, and then stored at −80° C. wrapped in plastic wrap. A cryostat was used to prepare 6 μm sections. Alternatively, the cell pellets were fixed overnight at 4° C. in freshly prepared 4% paraformaldehyde, processed through a series of ethanol/Hemo DE washes, and embedded in paraffin. A microtome was used to prepare 5 μm sections. Sections prepared from paraffin embedded pellets were stained with alcian blue using the following procedure. Samples were cleared and hydrated, stained with 1% alcian blue (either in 3% acetic acid, pH 2.5 or in 10% sulfuric acid, pH 0.2) for 30 minutes, washed three times with ddH₂O, dehydrated with alcohols, and cleared in HemoDE. Frozen sections were stained for collagen type II using the Vectastain Elite ABC kit according to the manufacturer's instructions (Vector Laboratories), except that samples were treated with 3% H₂O₂ in methanol for 30 minutes following hydration and then with 50 μU/ml chondroitinase ABC for 30 minutes. Anti-collagen type II antibody (NeoMarkers, Lab Vision Corp.; Fremont, Calif.) was used at a 1:50 dilution and the secondary biotinylated antibody was used at 1:200. Samples were counterstained with hematoxylin. Hypertrophic chondrocytes were induced as described (Mackay et al., 1998) and the pellets were stained with alcian blue and for collagen type X (1:50; NeoMarkers, Lab Vision Corp.).

Adipogenic Potential

To assess adipogenic potential, cells were cultured for up to 20 days in GM containing 2 μg/ml doxycycline, 50 μg/ml ascorbic acid 2-phosphate, 10 mM β-glycerophosphate, and 10⁻⁶ or 10⁻⁷ M dexamethasone. Medium was changed every two days and cultures were monitored for morphological signs of adipogenic differentiation. At 14-19 days following induction of differentiation, the cells were fixed with 10% neutral buffered formalin for 5 minutes, rinsed three times with ddH₂O, stained with either 0.3% w/v Oil Red 0 for 7 minutes or 100 ngiml Nile Red for 5 minutes, and rinsed three times with ddH₂O. Cells stained with Oil Red O were counterstained with hematoxylin for 2 minutes, rinsed three times in tap water, and once in ddH₂O. Cells stained with Nile Red were observed with fluorescent microscopy using a rhodamine or FITC filter.

Osteogeinic Potential

Osteogenic potential was assessed in the presence of 2 μg/ml doxycycline (Jaiswal et al., 1997). Cells were stained for alkaline phosphatase according to manufacturer's instructions using Sigma Kit 85.

Myogenic Potential

Myogenic potential was assessed by morphological observation and immunofluorescence using an antibody that recognizes myogenin (see section entitled Immunofluorescent Studies). Myotubes were observed in cultures treated to assess adipogenic or osteogenic potential.

1.10 Zebrafish Animals and Fin Amputations

Zebrafish 3-6 months of age were obtained from EKKWill Waterlife Resources (Gibsonton, Fla.) and used for caudal fin amputations. Fish were anaesthetized in tricaine and amputations were made using a razor blade, removing one-half of the fin. Animals were allowed to regenerate for various times in water kept at 31-33° C.; these temperatures facilitate more rapid regeneration than more commonly used temperatures of 25-28° C. (Johnson and Weston, 1995). Fish were then anaesthetized and the fin regenerate was removed for analyses.

1.11 Whole Mount In Situ Hybridization of Zebrafish

Probes

To generate antisense RNA probes with a dioxigenin labeling kit (Boehringer Mannheim), a 2.8 kb fgfr1 cDNA fragment, a 1.7 kb fgfr2 cDNA fragment, a 0.6 kb fgfr3 cDNA fragment, a 1.5 kb fgfr4 cDNA fragment (Thisse et al., 1995), a 1.2 kb msxb cDNA fragment, a 2.0 kb msxc cDNA (Akimenko et al., 1995), a 0.6 kb fgf8(ace) cDNA fragment (Reifers et al., 1998), a 2.2 kb fgf4.1 cDNA (Draper et al., 1999), a 2.4 kb wfgf cDNA (Draper et al., 1999), a 3.8 kb β-catenin cDNA (Kelly et al., 1995), a 2.6 kb flk1 cDNA fragment (Liao et al., 1997), and a 1.8 kb shh cDNA (Krauss et al., 1993) were used. Fragments containing zebrafish fgfr cDNA sequences were isolated by degenerate PCR using known fgfr tyrosine kinase domain sequences of other species. The assignment of fgfr genes was based on homology comparisons; these sequences have been deposited in Genbank.

In Situ Hybridization

Fin regenerates were fixed overnight at 4° C. in 4% paraformaldehyde in phosphate-buffered saline (PBS), washed briefly in 2 changes of PBS, and transferred to methanol for storage at −20° C. Fins were rehydrated stepwise through ethanol in PBS and then washed in 4 changes of PBS-0.1% polyoxyetbylenesorbitan monolaurate (Tween-20; PBT). Then, fins were incubated with 10 μg/ml proteinase K in PBT for 30 minutes and rinsed twice in PBT before 20 minutes refixation. After five washes with PBT, fins were prehybridized at 65° C. for one hour in buffer consisting of 50% formamide, 5× SSC (750 mM NaCl, 75 mM sodium citrate, pH 7.0), 0.1% Tween-20, 50 μg/ml heparin, and 500 μg/ml yeast RNA (pH to 6.0 with citric acid), and then hybridized overnight in hybridization buffer including 0.5 μg/ml dioxigenin-labeled RNA probe. Fins were washed at 65° C. for 10 minutes each in 75% hybridization buffer/25% 2× SSC, 50% hybridization buffer/50% 2× SSC, and 25% hybridization buffer/75% 2× SSC, followed by 2 washes for 30 minutes each in 0.2× SSC at 65° C. Further washes for 5 minutes each were done at room temperature in 75% 0.2× SSC/25% PBT, 50% 0.2x SSC/50% PBT, and 25% 0.2× SSC/75% PBT. After a one hour incubation period in PBT with 2 mg/ml bovine serum albumin, fins were incubated for 2 hours in the same solution with a 1:2000 dilution of fin-preabsorbed, anti-dioxigenin antibody coupled to alkaline phosphatase (Boehringer Mannheim). For the alkaline phosphatase reaction, fins were first washed 3 times in reaction buffer (100 mM Tris-HCl pH 9.5; 50 mrM MgCl₂, 100 mM NaCl, 0.1% Tween-20, 1 mM levamisol) and then incubated in reaction buffer with 1× nitro blue terazolium/5-bromo-4-chloro-3-indolyl-phosphate (NBT/BCIP) substrate. In general, positive signals were obtained in 0.5-3 hours. Following the staining reaction, fins were washed in several changes of PBT and fixed in 4% paraformaldehyde in PBS. To obtain sections of fin regenerates, fins were first mounted in 1.5% agarose/5% sucrose and then incubated in 30% sucrose overnight. Frozen blocks were sectioned at 14 μm and observed using Nomarski optics.

For each probe, at least 7 fins were examined for expression at 0, 6, 12, 18, 24, 48, and 96 hours post-amputation. For 18, 24, and 48 hour timepoints with fgfr1, msxb, msxc, and wfgf probes, 25-100 fins were examined in several different experiments. Experiments with sense strand RNA probes were performed with initial antisense experiments to estimate the specificity of signals. To assess gene expression in pharmacologically treated fins, an equal number of untreated fins were also examined. Then, all staining reactions were stopped after strong signals were seen in untreated fins under low magnification.

1.12 Fgfr1 Inhibitor Treatments in Zebrafish

SU5402 (Rᵢ; SUGEN, South San Francisco, Calif.) was dissolved in dimethylsulfoxide (DMSO) and added to fish water at a final concentration of 1.7 μM or 17 μM (0.01% DMSO). Up to 10 fish were treated in one liter of water, and tanks were maintained in the dark at 31-33° C. with SU5402 solutions replaced every 24 hours. Zebrafish survived normally and demonstrated no unusual behavior while in the inhibitor solution.

1.13 BrdU Incorporation in Zebrafish

BrdU was dissolved in PBS and fish were treated at a final concentration of 100 μg/ml. For one experiment, fins were amputated and allowed to regenerate for 18 or 24 hours in the absence or presence of 17 μM Rᵢ, with BrdU present during the final 6 hours of regeneration. To test the effects of Rᵢ on proliferation in the established blastema, fins were first allowed to regenerate for 40 hours. Then, untreated fish regenerated an additional 2 hours before a 6 hour incubation with BrdU, while Rᵢ-treated fish underwent a 2 hour Rᵢpreincubation period before a 6 hour period with both Rᵢ and BrdU.

Fins were collected and fixed in 70% ethanol/2 mM glycine overnight, and 10 μm sections were made from frozen blocks. These sections were stained for BrdU incorporation using a detection kit (Roche; Basel, Switzerland), and counterstained with hematoxylin. Sections from untreated and Rᵢ-treated fins were simultaneously processed and developed. Approximately 100 sections from 8 fins were examined from 18 and 24 hour timepoint experiments, while approximately 50 sections from 6 fins were examined from the 48 hour timepoint experiment.

2.1 Regeneration Extract Induces Newt Myotubes to Dedifferentiate

To determine if factors contained in regenerating newt tissue can induce cellular morphologic changes indicative of dedifferentiation, a regenerating newt limb extract (RNLE) was prepared, applied to cultured newt myotubes, and the myotubes followed with light microscopy.

Wound epithelium and proximally-adjacent tissues from day 1-5 newt limb regenerates were used to prepare RNLE as described above. A1 myotubes were cultured at very low density (<0.25 cell/hpf) in DM with 0.3 mg/ml RNLE, and each individual myotube was followed closely for 10 days and photographed every 12-24 hours. The first signs of morphologic dedifferentiation were evident on day 3 when myotubes altered their shape and cleaved into smaller myotubes. By day 10, 16% of the myotubes cleaved to form smaller myotubes or mononucleated cells (Table II). No morphological changes or cellular cleavage was seen in myotubes cultured in DM alone or in DM plus non-regeneration limb extract (negative controls). These findings indicate that RNLE can induce morphologic dedifferentiation in cultured newt myotubes.

To determine the effect of RNLE on normally quiescent multinucleated newt myotubes, RNLE was applied to the cells and tested for BrdU incorporation to assay DNA synthesis. Newt A1 myotubes were plated at low density (1-2 cells/hpf) in D)M and cultured with 0.3 mg/ml RNLE on day 0. Medium and extract were changed daily and myotubes were assayed for BrdU incorporation on day 4. When quiescent newt A1 myotubes were cultured in DM with RNLE, 25% of the cells were stimulated to enter the S phase of the cell cycle (Table II). By contrast, only 2% of myotubes cultured in DM alone and 3% in DM with 0.3 mg/ml non-regenerating extract incorporated BrdU. These data indicate that regenerating newt tissue contains factors that can induce newt myotubes to reenter the cell cycle.

TABLE II
Newt myotube dedifferentiation induced by RNLE

| Media | MD¹ | BrdU² |
|---|---|---|
| Lysate | 9/56 (16%) | 25/102 (25%) |
| DM w/non-RNLE | 0/50 (0%) | 2/59 (3%) |
| DM alone | 0/43 (0%) | 2/96 (2%) |

¹Morphological dedifferentiation, indicated by cleavage of multinucleated myotubes into smaller myotubes and/or in proliferating mononucleated cells.
²BrdU incorporation to determine entry into S phase

2.2 RNLE Induces Molecular and Cellular Dedifferentiation of Mammalian Myotubes

To determine if RNLE contains factors that can induce morphologic dedifferentiation of mammalian myotubes, RNLE was applied to C2C12 myotubes and the cells followed by light microscopy.

The myotubes were plated at very low density (<0.25 cell/hpf), cultured in DM with 0.3 mg/ml RNLE on day 0, and individually photographed every 12-24 hours to document cellular morphologic changes that occurred over the next 10 days. The medium and extract were changed daily. Cellular cleavage was noted by day 2-3 in 11% of the myotubes plated, and cleavage was followed by cellular proliferation in half of these myotubes (Table III). These cellular phenomena were not seen in any C2C12 myotubes cultured with DM alone or DM with 0.3 mg/ml non-RNLE. Thus, murine myotubes cultured with RNLE undergo cytokinetic cleavage to smaller myotubes at nearly the same frequency as newt myotubes (11% vs. 16%). In addition, cleavage was often followed by cellular proliferation in the C2C12 myotubes, an unexpected finding since RNLE-treated newt myotubes did not proliferate. These data indicate that RNLE induces dedifferentiation and proliferation of cultured mammalian myotubes.

To determine if RNLE affects expression of muscle determination and differentiation proteins, RNLE was applied to C2C12 myotubes and indirect immunofluorescence assays were performed to determine altered expression of the muscle differentiation proteins myogenin and myoD and of the muscle contractile protein, troponin-T. Each of these myogenic markers was downregulated in C2C12 myotubes when cultured with the RNLE for four days. Nuclear downregulation of myogenin and MyoD was seen respectively in 15% and 19% of the myotubes. Troponin-T was downregulated in the cytoplasm of 30% of the myotubes. By contrast, myoD and myogenin were consistently present in the controls, and troponin-T was identified in approximately 94-97% of the controls (Table III). Downregulation of all markers in RNLE-treated myotubes was greatest by day 4. These data indicate that newt RNLE downregulates skeletal muscle differentiation factors in cultured mammalian myotubes.

To determine if regenerating newt tissue could induce S phase reentry in terminally differentiated mammalian myotubes, BrdU incorporation was assayed in RNLE treated C2C12 myotubes. C2C12 myotubes were plated at low density (1-2 cells/hpf) and cultured in DM with 0.3 mg/ml of the RNLE. The extract was added on day 0, medium and extract were changed daily, and cells were assayed for BrdU incorporation on the fourth day. Eighteen percent of RNLE-treated C2C12 myotubes showed S phase reentry (FIG. 3, Table 1B). By contrast, no BrdU incorporation was seen in cells cultured in DM alone or in DM with non-RNLE (Table II). RNLE can therefore induce cell cycle reentry in cultured mammalian myotubes.

TABLE III
Mammalian myotube dedifferentiation induced by RNLE

| Media | MD¹ | BrdU² | MyoD³ | Myogenin³ | Troponin-T³ |
|---|---|---|---|---|---|
| Lysate | 10/92 (11%) | 14/76 (18%) | 18/93 (19%) | 12/82 (15%) | 20/66 (30%) |
| DM w/non-RNLE | 0/63 (0%) | 0/30 (0%) | 0/46 (0%) | 0/54 (0%) | 1/32 (3%) |
| DM alone | 0/61 (0%) | 0/32 (0%) | 0/40 (0%) | 0/48 (0%) | 3/47 (6%) |

¹Morphological dedifferentiation, indicated by cleavage of multinucleated myotubes into smaller myotubes and/or in proliferating mononucleated cells.
²BrdU incorporation to determine entry into S phase
³Downregulation of muscle cell-specific markers compared to untreated myotubes. Cells were stained on the fourth day of the experiment.

2.3 Dedifferentiation Signal is Likely Comprised of Proteins

The dedifferentiation signal(s) found in the RNLE could belong to a number of different types of biomolecules, including proteins, lipids, nucleic acids, and polysaccharides. To characterize the nature of one or more of the active components of the RNLE, the inventors subjected the extract to a number of different conditions. The results are summarized in Table TV.

The preparation of RNLE reduced the likelihood that the dedifferentiation factor(s) were lipids, since nonsoluble lipids were removed following a high-speed centrifugation step. Repeated freezing and thawing of RNLE reduced the dedifferentiation activity, while boiling for 5 minutes eradicated all activity. When the RNLE was treated with the protease, trypsin, the dedifferentiation signal was abolished, indicating that proteins were a primary component of the factor. The dedifferentiation signal may comprise a single protein or a group of proteins; such proteins may contain certain post-translational modifications, e.g. glycosylation.

TABLE IV
RNLE active component characterization
by measuring effect on S
phase reentry

| Treatment | BrdU |
|---|---|
| Heat inactivation¹ | inhibition |
| Freeze/thaw | inhibition |
| Protease² | inhibition |
| SU5402 (Rᵢ)³ | no effect |

¹100° C. for 5 minutes,
²10% trypsin,
³Inhibits Fgfr.

2.4 Generation of C2C12 Clones Containing an Inducible msx1 Gene

The mouse msx1 gene (SEQ ID NO: 1) (Hill et al., 1989) was cloned into the LINX vector in both the forward (LINX-msx1-fwd) and reverse (LINX-msx1-rev) orientations. LINX is a retroviral vector containing a minimal CMV promoter regulated by the tetracycline-controlled transactivator (tTA) (Gossen and Bujard, 1992; Hoshimaru et al., 1996). Tetracycline or its analog, doxycycline (dox), binds to and inactivates tTA, preventing transcription from the minimal CMV promoter. In the absence of these antibiotics, tTA binds to the tetracycline response element (TRE) and induces transcription.

LINX-msx1-fwd and LINX-msx1-rev were transduced into C2C12 myoblasts and clones (Fwd-2, Fwd-3, and Rev-2) grown in selective medium were either induced or suppressed for msx1 expression, using dox. Total RNA was extracted and Northern blots were probed with a 40-nucleotide oligomer complimentary to the msx1 transcript. Msx1 was induced, suppressed, or induced and then suppressed. After five days of induction, a 2.1 kb msx1 signal was observed in C2C12-LINX-msx1-fwd (Fwd) clones. Phosphorimage analysis revealed a 25-fold induction in msx1 expression. Inducible expression can be reversed when msx1 was again suppressed by growth in medium containing 2 μg/ml doxycycline. C2C12 myoblasts and clones containing the LINX-msx1-rev construct (Rev) did not express msx1.

Ectopic expression of msx1 has been shown to inhibit the differentiation of mouse myoblasts into myotubes (Song et al., 1992). To assess whether induced msx1 protein was functional, the transfected myoblasts were tested for their ability to differentiate. Clones were grown in dox to either induce or suppress msx1 expression. Once confluency was reached, GM was replaced with DM, and induction or suppression of msx1 was continued. Over ten days, the clones were observed for morphological signs of differentiation by phase contrast microscopy. Fwd clones that were cultured in conditions that suppressed msx1 expression readily produced myotubes, while those expressing msx1 failed to produce myotubes. Control C2C12 myoblasts and Rev clones differentiated normally when treated with the induction or suppression conditions. These results indicate that the Fwd clones contained an inducible msx1 gene that produces functional msx1. Two Fwd clones (Fwd-2 and Fwd-3) and one Rev clone (Rev-2) were chosen for further study.

2.5 Msx1 Reverses Expression of Muscle Differentiation Proteins in Mouse Myotubes

One biochemical indicator of myotube dedifferentiation would be the reduction in levels of myogenic differentiation proteins. To determine if the myogenic factors MyoD, myogenin, MRF4, and p21 are reduced as a consequence of msx1 expression, indirect immunofluorescence assays were performed on myotubes that had been induced to express msx1 in the presence of GM. All of these myogenic factors were reduced to varying degrees in murine myotubes. Within 1 day of msx1 induction, MRF4 was reduced to undetectable levels in 34% of induced myotubes. Likewise, myogenin was undetectable in approximately 26% of all induced myotubes. The percentage of myotubes showing undetectable levels of MRF4 and myogenin rose through days 2 and 3 to 50% and 38%, respectively. MyoD expression was not affected until.the second day of msx1 induction. On day 2, 10% of all myotubes exhibited a marked reduction of MyoD levels and this percentage rose to 28% by day 3. The percentage of myotubes exhibiting undetectable levels of p21 rose from 10% on day 1 postinduction to 20% by day 3. To ensure that the observed reduction of myogenic protein levels of test myotubes was not the result of myotube aging, control myotubes were matched for age. Normal expression of muscle proteins was observed in 90%-100% of control C2C12 myotubes. These results indicate that ectopic msx1 expression can cause a reduction in the levels of myogenic proteins in terminally differentiated mammalian myotubes.

2.6 Msx1 Induces Mouse Myotube Cleavage and Cellular Proliferation

To test whether ectopic Y7msx1 expression and growth factor stimulation could induce cleavage of terminally differentiated mammalian myotubes, isolated myotubes were plated at low density, and the remaining mononucleated cells were eliminated by lethal injection and/or needle ablation (Kumar et al., 2000). Fresh DM was added to the myotubes, and they were incubated overnight. The cultures were again examined for residual morionucleated cells and those present were eliminated before photographing the entire gridded region. No residual mononucleated cells were observed following this procedure in either Fwd or control myotubes. msx1 expression was then induced in one set of Fwd myotubes, while a control set of myotubes remained suppressed. Both sets of myotubes were stimulated with GM and followed daily for up to 7 days by microscopic observation and photography. Dedifferentiation was assessed by morphologic examination using the following criteria: (1) cleavage of the myotubes into mononucleated cells or smaller myotubes, and (2) proliferation of the myotube-derived manonucleated cells. FIG. 3A shows an example of a large multinucleated myotube that cleaved to form two smaller multinucleated myotubes. Cleavage of this large myotube was almost complete at day 6 at msx1 induction. Once cleaved, the two myotubes remained separated and viable through the duration of the dedifferentiate served in control myotube cultures. Of the 148 test myotubes treated with the induction conditions, 13 (8.8%) underwent cleavage to form either smaller myotubes or mononucleated cells. The first signs of dedifferentiation were evident two days following induction of msx1. At this time, the dedifferentiating myotubes had completely cleaved to form mononucleated cells. Signs of impending cleavage were also observed, such as cell stretching and cleavage initiation. Such cleavages eventually produced proliferating, mononucleated cells by day 4.5. The mononucleated cells arising from these myotubes continued to proliferate and reached cellular confluence by day 7. Proliferation of the resulting mononucleated cells was evident by day 5, and on day 6, numerous myotube-derived mononucleated cells were present. Of 148 test myotubes treated with the induction conditions, 8 (5.4%) dedifferentiated to a pool of proliferating mononucleated cells. Thus, msx1 can induce myotubes to stretch and cleave, giving rise to smaller myotubes or mononucleated cells that proliferate.

To ensure that myotube cleavage to mononucleated cells and subsequent proliferation resulted from msx1 expression and was not an artifact of hidden, reserve mononucleated cells, these experiments were repeated, using control cells consisting of uninduced Fwd, Rev, and nontransduced C2C12 myotubes. Of the 151 control myotubes studied, only one atypical myotube cleaved to form a few mononucleated cells However, these cells did not proliferate even after 7 days in GM. No other control myotubes showed evidence of stretching and cleaving, and no proliferating mononucleated cells were observed. The Fisher-Irwin exact test indicates that the difference in cleavage frequency between msx1-expressing and control myotubes is significant at p=0.0006. Likewise, the difference in cleavage/proliferation frequency between msx1-expressing and control myotubes is significant at p=0.003. Thus the combination of ectopic msx1 expression and stimulation with growth factors can induce a percentage of mouse myotubes to be dedifferentiate into smaller myotubes or proliferating, mononucleated cells.

2.7 Msx1 Induces Dedifferentiation of Mouse Myotubes to Pluripotent Stem Cells

To determine if the dedifferentiated, proliferating mononucleated cells were pluripotent, two clonal populations of cells derived from a single Fwd-2 myotube were isolated. The clones were cultured under conditions that were favorable for adipogenesis, chondrogenesis, osteogensis, or myogenensis (Dennis et al., 1999; Grigoriadis et al., 1988; Jaiswal et al., 1997; Mackay et al., 1998; Pittenger et al., 1999). Msx1 expression was suppressed during these redifferentiation assays.

The dedifferentiated clones were tested for chondrogenic potential by pelleting 2.5×10⁵ cells in chondrogenic differentiation medium and feeding the cell pellets every two days with fresh medium. These cells readily differentiated into chondrocytes that produced an extracellular matrix staining faintly with alcian blue and containing collagen type II. Differentiated cells could be further induced to form hypertrophic chondrocytes that stained with alcian blue and reacted with type X collagen. No chondrocytes or hypertrophic chondrocytes were identified in control C2C12 or msx1-rev-2 cells.

When cultured in adipogenic differentiation medium (ADM) for 7-16 days, the dedifferentiated clones produced cells that exhibited adipocyte morphology. These cells were characterized by the presence of numerous vacuoles that stained bright orange upon treatment with the lipophilic dyes, oil red O and Nile red (FIG. 4A). Control C2C12 or Rev-2 cells that had been treated with ADM did not show these characteristic features of adipogenesis (FIG. 4A). The combination of morphologic features and lipid-staining vacuoles suggests that some of the cells had differentiated into adipocytes.

Dedifferentiated clones could also be induced to differentiate into cells expressing an osteogenic marker by treatment with osteogenic-inducing medium (OIM). We observed numerous cell foci per 35 mm plate that stained positive for alkaline phosphatase activity, while very little alkaline phosphatase was identified in control C2C12 or Rev-2 cells (FIG. 4A). Myotubes readily formed in ADM or OIM and were identified by morphology and reactivity to an anti-myogenin antibody (FIG. 4A). As expected, control C2C12 and Rev cells also readily differentiated into myotubes (FIG. 4A; data not shown).

Thus, the combination of ectopic msx1 expression and growth factor treatment can induce terminally-differentiated mouse myotubes to dedifferentiate to a pool of proliferating, pluripotent stem cells that are capable of redifferentiating into several cell lineages.

2.8 Msx1 Induces Transdetermination of Mouse Myoblasts

The inventors contemplated that if msx1 expression caused terminally-differentiated myotubes to completely dedifferentiate, ectopic expression of msx1 might promote transdetermination of C2C12 myoblasts. Msx1 expression was induced in Fwd myoblasts for five days and then suppressed. When treated with the appropriate media, these cells readily differentiated into chondrocytes, adipocytes, myotubes, and cells expressing an osteogenic marker (FIG. 5). No evidence of transdetermination was observed in control cells. These results indicate that transdetermination of myoblasts resulted from ectopic expression of msx1.

2.9 Expression of Fgf Signaling Pathway Members During Zebrafish Fin Blastema Formation and Regenerative Outgrowth

The zebrafish fin is composed of several segmented bony fin rays, or lepidotrichia, each consisting of a pair of concave, facing hemirays that surround connective tissue, including fibroblasts, as well as nerves and blood vessels. Lepidotrichia are connected by vascularized and innervated soft mesenchymal tissue. The early events that occur during lepidotrichium regeneration can be separated into four stages (A-D) when raised at 33° C. (Goss and Stagg, 1957; Johnson and Weston, 1995; Santamaria and Becerra, 1991). During the first stage (0-12 hours after amputation), a wound epidermis derived from fin epidermal cells forms over the stump. During stage B (approximately 12-24 hours after amputation), wound epidermal cells continue to accumulate. Meanwhile, fibroblasts and scleroblasts (or osteoblasts) located 1-2 segments proximal to the amputation site and between hemirays loosen and disorganize, assume a longitudinal orientation, and appear to migrate toward the wound epidermis. By stage C (24-48 hours), distal migration and proliferation of these cells have resulted in a blastema. During stage D (48 hours and throughout the remainder of regeneration), the blastema is thought to have two prominent functions: (1) the distal portion facilitates outgrowth via cell division; (2) the proximal portion differentiates to form specific structures of the regenerating fin. Following caudal fin amputation, complete regeneration occurs in 1-2 weeks.

To demonstrate that Fgf signaling participates in zebrafish caudal fin regeneration, the expression of four fgfr genes in the early fin regenerate at timepoints ranging from 0 to 96 hours postamputation was assessed using sin situ hybridization. The earliest point at which faint but consistent expression of fgfr1 was detected in fin regenerates was 18 hours postamputation, in cells that appeared to be in the process of forming the blastema. Longitudinal fin sections indicated that, at 18-24 hours postamputation, fgfr1 transcripts localize in fibroblast-like cells between hemirays just proximal and distal to the amputation plane. At 48 hours postamputation, during regenerative outgrowth, whole mount analyses consistently revealed expression of fgfr1 in both distal and proximal portions of the regenerate. Sections at this stage indicated transcripts in a small population of cells comprising the distal blastema, as well as in a significant portion of the basal layer of the regeneration epidermis. The epidermal domain appeared to overlap with cells that express sonic hedgehog (shh) at this stage (Laforest et al., 1998). These expression domains were also conspicuous at 96 hours postamputation. In addition, weak but consistent expression of fgfr2 and fgfr3 was observed in the proximal fin regenerate as early as 48 hours after amputation. These receptors were similarly expressed in diffuse domains. fgfr4 expression was not detected in the regenerating fin. These data indicate that cells of the fin regenerate, including blastemal progenitor cells as well as mature blastemal cells, express receptors for Fgfs.

Because msx genes have been implicated as downstream transcriptional targets in Fgf signaling pathways (Kettunen and Thesleff, 1998; Vogel et al., 1995; Wang and Sassoon, 1995), and have been postulated to be important for the undifferentiated state of embryonic mesenchymal tissue (Song et al., 1992), as well as the adult urodele limb blastema (Koshiba et al., 1998), the onset and domain of expression of zebrafish msxb and msxc in the fin regenerate was examined. Detectable msxb expression in fin regenerates was 18 hours postamputation. Sections indicated that during blastema formation, msxb transcripts were distributed in a similar manner as fgfr1 transcripts, in fibroblast-like cells just proximal and distal to the amputation plane. By 48 hours and throughout the remainder of regeneration, all msxb-positive cells were contained within the distal blastemal region, as previously reported (Akimenko et al., 1995). Msxc expression domains were virtually identical to those of msxb at all timepoints. Colocalization of fgfr1 transcripts with msxb and msxc transcripts during blastema formation and regenerative outgrowth supports the hypothesis that Fgf signaling is important for these processes.

To demonstrate that Fgfs are synthesized in the regenerating fin, probes representing characterized zebrafish fgf genes were used for in situ hybridization experiments. No fgf4.1 or fgf8 (ace) transcripts were detected in fin regenerates. However, a member of the Fgf8, Fgf17, and Fgf18 subclass of Fgf ligands, “Wound (W)fgf”, was expressed in the fin regenerate (Draper et al., 1999). wfgf expression was consistently observed at 48 hours postamputation in the distal-most cells of the regeneration epidermis, where it was maintained throughout outgrowth. Experiments examining wfgf expression during blastema formation were equivocal, showing faint expression in approximately 50% of the regenerates. These data indicate that at least one Fgf member is present in the regenerating fin.

2.10 Inhibition of Fgfr1 Blocks Blastema Formation

To functionally assess roles of Fgfs in fin regeneration, the lipophilic drug SU5402 (Rᵢ) which has been shown to disrupt Fgfr1 autophosphorylation and substrate phosphorylation by binding specifically to its tyrosine kinase domain, was used. The IC₅₀ of Rᵢ with respect to Fgfr1 activity in mammalian cells was shown previously to be 10-20 μM (Mohammadi et al., 1997). This concentration of Rᵢ causes a dramatic truncation of posterior structures when applied to developing zebrafish embryos. Such embryos appear remarkably similar to those injected with mRNA encoding a dominant-negative Fgfr1 (Griffin et al., 1995). Therefore, Rᵢ effectively blocked zebrafish Fgfr1 activity.

Previous studies have shown that Rᵢ does not block platelet-derived growth factor, epidermal growth factor, and insulin receptors at concentrations greater than 50 μM in mammalian cells, and has no effects on activities of numerous serine threonine kinases (Mohammadi et al., 1997; Sun et al., 1999). However, Rᵢ does inhibit Flk1, a vascular endothelial growth factor receptor and the earliest known marker for endothelial progenitor cells (Liao et al., 1997), at 10-20 μM. In zebrafish fin regenerates, consistent expression of flk1 was not observed until 96 hours postamputation, when it appeared in blastemal cells (n=22). flk1 expression was not apparent during blastema formation by in situ hybridization 24 hours postamputation (n=14).

To determine if signaling through Fgfr1 is required for regeneration, zebrafish were treated for 96 hours with Rᵢ immediately following amputation. Treatment of zebrafish with 1.7 μM Rᵢ (0.5 mg/liter) inhibited fin regeneration to varying degrees. Of 10 fins examined, 4 regenerated normally, 5 showed slight regenerative defects, and one had a regenerative block. However, all animals exposed to 17 μM Rᵢ (5 mg/liter) demonstrated complete regenerative blocks (n=9). These results indicated that Fgf signaling is required for zebrafish fin regeneration.

To determine if a blastema forms in the absence of Fgf signaling, Rᵢ-treated fin regenerates were examined morphologically. While a wound epidermis consistently formed over the fin stumps of Rᵢ-treated fish, blastemal morphogenesis did not occur. However, mesenchymal cells proximal to the amputation plane showed disorganization, as well as longitudinal orientation suggestive of distal migration.

BrdU incorporation was used to analyze DNA replication and cellular proliferation. Normal proximal mesenchymal cell labeling in Rᵢ-treated fins during 12-18 hours and 18-24 hours postamputation was observed. To determine if blastemal cells underwent DNA replication in the presence of Rᵢ, BrdU incorporation in fins briefly treated with Rᵢ during regenerative outgrowth (40-48 hours postamputation) was analyzed. Blastemal cells of these fins demonstrated greatly reduced incorporation of BrdU. While distal blastemal cells were routinely labeled in sections of untreated fins, labeling of these cells was never observed in sections from Rᵢ-treated fins. Furthermore, labeled proximal blastemal cells, which likely had incorporated BrdU through division in the distal blastema, were heavily distributed in sections of untreated fins but sparsely distributed in sections of Rᵢ-treated fins. Nevertheless, proliferation in mesenchymal cells proximal to the amputation plane again was similar in untreated and Rᵢ-treated groups. The lack of effect by Rᵢ on proximal mesenchymal tissue was not due to poor tissue penetration, as fins treated for 48 hours with Rᵢ before BrdU treatment also showed normal proximal mesenchymal incorporation. These results indicate that Fgf signaling is essential for blastema formation, likely by facilitating mesenchymal cellular proliferation near the wound epidermis.

To assess molecular effects of the regenerative block in Rᵢ-treated fins, the expression of β-catenin, msxb, and msxc was analyzed. β-catenin was expressed at high levels in the wound epidermis of untreated regenerating fins as early as 3 hours postamputation and throughout the regeneration process. β-catenin expression was normal in Rᵢ-treated fins, suggesting that such fins have no gross defects in wound healing (n=7). However, expression of the blastemal markers msxb and msxc in Rᵢ-treated fins was extremely low or undetectable in 24 hour regenerates, and undetectable in 48 hour regenerates (msxb: 21 fins, msxc: 8 fins). These data indicate that Fgf signaling is necessary for msxb/c transcription in the fin regenerate.

2.11 Fgfr1 Inhibition Blocks Regenerative Outgrowth

Because wfgf and fgfr1 expression domains were maintained in the fin regenerate during outgrowth, and as blastemal cell BrdU incorporation was blocked by Rᵢ, Fgf signaling likely participates in blastemal maintenance/regenerative outgrowth. To test this hypothesis, the effects of Rᵢ on ongoing regenerates as examined. Rᵢ treatment inhibited further outgrowth of 24-72 hour fin regenerates and often caused the accumulation of an unusually thick regeneration epidermis, as well as dorsoventral migration of melanocytes into adjacent rays. This result may be a consequence of cellular migratory processes by the epidermal and pigment cells that usually pair with new distal growth. In addition, new bone deposition was not interrupted by Rᵢ treatment despite the lack of outgrowth, as lepidotrichial material was observed at unusually distal locations in sections of these fins.

To investigate the molecular effects of this outgrowth inhibition by Rᵢ, marker expression was examined following a 24 hour Rᵢ application period. No significant reduction of 48 or 72 hour epidermal wfgf expression was seen (n=16). However, expression of msxb was diminished in Rᵢ-treated fins that had already regenerated normally for 24 or 48 hours (10 of 18 Rᵢ-treated fins had no detectable msxb expression, while the remaining 8 fins showed low levels). Similar effects on msxc expression were observed (n=8) msxb expression was not detected in 24 or 48 hour fin regenerates exposed to Rᵢ for 48 hours (n=1S). Thus, Fgf signaling is required for blastema maintenance and regenerative outgrowth, but is not crucial for other processes including melanocyte migration or bone deposition.

Finally, because fgfr1 also was expressed in epidermal cells during regenerative outgrowth (see FIG. 2C, D), Fgf signaling may be important for patterning the regenerate. To test this hypothesis, the effects of Rᵢ treatment on expression of the patterning gene shh were determined. As previously reported, shh localized to bilateral domains of the basal layer of the fin epidermis as early as 48 hours postamputation (Laforest et al., 1998). Release of Shh from these cells is thought to direct differentiation of blastemal cells into scleroblasts, which deposit bone in forming the new segments of the regenerate. Treatment of 48 or 72 hour fin regenerates with Rᵢ for 24 hours dramatically reduced shh expression (0 of 18 fins had detectable s/h/h transcripts; FIG. 6H). These data indicate that intact Fgf signaling is required for normal expression of shh in the fin regenerate.

References

U.S. Pat. No. 4,166,452. Apparatus for testing human responses to stimuli. 1979.

U.S. Pat. No. 4,816,567. Recombinant immunoglobin preparations. 1989.

WO 90/10448. Covalent conjugates of lipid and oligonucleotide. 1990.

EPO 402226. Transformation vectors for yeast Yarrowia. 1990.

WO 91/04753. Conjugates of antisense oligonucleotides and therapeutic uses thereof. 1991.

WO 91/00357. New strain with filamentous fungi mutants, process for the production of recombinant proteins using said strain, and strains and proteins. 1991.

WO 91/06629. Oligonucleotide analogs with novel linkages. 1991.

Akimenko, M. A., S. L. Johnson, M. Westerfield, and M. Ekker. 1995. Differential induction of four msx homeobox genes during fin development and regeneration in zebrafish. Development. 121:347-357.

Alam, J., and J. L. Cook. 1990. Reporter genes: Application to the study of mammalian gene transcription. Anal. Biochem. 188:245-254.

Altschul, S. F., and W. Gish. 1996. Local alignment statistics. Methods Enzymol. 266:460-80.

Altschul, S. F., T. L. Madden, A. A. Schaffer, J. Zhang, et al. 1997. Gapped BLAST and PSI-BLAST: a new generation of protein database search programs. Nucleic Acids Res. 25:3389-402.

Andres, V., and K. Walsh. 1996. Myogenin expression, cell cycle withdrawal, and phenotypic differentiation are temporally separable events that precede cell fusion upon myogenesis. J Cell Biol. 132:657-666.

Austin, C. P., and C. L. Cepko. 1990. Cellular migration patterns in the developing mouse cerebral cortex. Development. 110:713-732.

Ausubel, F. M., R. Brent, R. E. Kingston, D. D. Moore, et al. 1987. Current protocols in molecular biology. John Wiley & Sons, New York.

Baird, A., and M. Klagsbrun. 1991. The fibroblast growth factor family. New York Academy of Sciences, New York, N.Y.

Bechtold, N., and G. Pelletier. 1998. In planta Agrobacterium-mediated transformation of adult Arabidopsis thaliana plants by vacuum infiltration. Methods Mol Biol. 82:259-66.

Becker, D. M., and L. Guarente. 1991. High-efficiency transformation of yeast by electroporation. Methods Enzymol. 194:182-187.

Becker, R. O., S. Chapin, and R. Sherry. 1974. Regeneration of the ventricular myocardium in amphibians. Nature. 248:145-147.

Beggs, J. D. 1978. Transformation of yeast by a replicating hybrid plasmid. Nature. 275:104-109.

Berger, J., J. Hauber, R. Hauber, R. Geiger, et al. 1988. Secreted placental alkaline phosphatase: A powerful new quantitative indicator of gene expression in eukaryotic cells. Gene. 66: 1-10.

Bodine, D. M., K. T. McDonagh, N. E. Seidel, and A. W. Nienhuis. 1991. Survival and retrovirus infection of murine hematopoietic stem cells in vitro: effects of 5-FU and method of infection. Exp. Hematol. 19:206-212.

Brockes, J. P. 1997. Amphibian limb regeneration: rebuilding a complex structure. Science. 276:81-7.

Capecchi, M. R. 1980. High efficiency transformation by direct microinjection of DNA into cultured mammalian cells. Cell. 22:479.

Carter, P. 1986. Site-directed mutagenesis. Biochem J. 237:1-7.

Case, M. E., M. Schweizer, S. R. Kushner, and N. H. Giles. 1979. Efficient transformation of Neurospora crassa by utilizing hybrid plasmid DNA. Proc Natl Acad Sci USA. 76:5259-63.

Cepko, C. L., B. E. Roberts, and R. E. Mulligan. 1984. Construction and applications of a highly transmissible murine retrovirus shuttle vector. Cell. 37:1053-1062.

Chalfie, M., Y. tu, G. Euskirchen, W. W. Ward, et a. 1994. Green fluorescent protein as a marker for gene expression. Science. 263:802-805.

Chaney, W. G., D. R. Howard, J. W. Pollard, S. Sallustio, et al. 1986. High-frequency transfection of CHO cells using Polybrene. Somatic Cell Mol. Genet. 12:237.

Chen, C., and H. Okayama. 1988. Calcium phosphate-mediated gene transfer: A highly efficient system for stably transforming cells with plasmid DNA. BioTechiniques. 6:632-638.

Chen, S. H., H. D. Shine, J. C. Goodman, R. G. Grossman, et al 1994. Gene therapy for brain tumors: regression of experimental gliomas by adenovirus-mediated gene transfer in vivo. Proc Natl Acad Sci USA. 91:3054-7.

Cohen, S. M. N., A. C. Y. Chang, and L. Hsu. 1972. Nonchromosomal antibiotic resistance in bacteria: Genetic transformation of Escherichia coli by R-factor DNA. Proc. Natl. Acad. Sci. USA. 69:2110.

Crossley, P. H., G. Minowada, C. A. MacArthur, and G. R. Martin. 1996. Roles for FGF8 in the induction, initiation, and maintenance of chick limb development. Cell. 84:127-36.

Davis, B. M., J. L. Ayers, L. Koran, J. Carlson, et al 1990. Time course of salamander spinal cord regeneration and recovery of swimming: HRP retrograde pathway tracing and kinematic analysis. Exp Neurol. 108:198-213.

de Louvencourt, L., H. Fukuhara, H. Heslot, and M. Wesolowski. 1983. Transformation of Kluyveromyces lactis by killer plasmid DNA. J Bacteriol. 154:737-42.

de Wet, J. R., K. V. Wood, M. DeLuca, D. R. Helinski, et al. 1987. Structure and expression in mammalian cells. Mol. Cell Biol. 7:725-737.

Demerec, M., E. A. Adelberg, A. J. Clark, and P. E. Hartman. 1966. A proposal for a uniform nomenclature in bacterial genetics. Genetics. 54:61-76.

Dennis, J. E., A. Merriam, A. Awadallah, J. U. Yoo, et al. 1999. A quadripotential mesenchymal progenitor cell isolated from the marrow of an adult mouse. J Bone Miner Res. 14:700-709.

Draper, B., J. Phillips, D. Stock, and C. Kimmel. 1999. FGF signaling and mesodermal patterning in the zebrafish embryo. Dev Biol. 210:151-180.

Elroy-Stein, O., and B. Moss. 1990. Cytoplasmic expression system based on constitutive synthesis of bacteriophage T7 RNA polymerase in mammalian cells. Proc. Natl. Acad. Sci. USA. 87:6743-6747.

Endo, T., and B. Nadal-Ginard. 1989. SV40 large T antigen induces reentry of terminally differentiated myotubes into the cell cycle. In Cellular and Molecular Biology of Muscle Development. F. Stockdale and L. Kedes, editors. Alan R. Liss, Inc., New York. 95-104.

Endo, T., and B. Nadal-Ginard. 1998. Reversal of myogenic terminal differentiation by SV40 large T antigen results in mitosis and apoptosis. J Cell Sci. 111: 1081-1093.

U.S. Pat. No.4,522,811. Serial injection of muramyldipeptides and liposomes enhances the anti-infective activity of muramyldipeptides Serial injection of muramyldipeptides and liposomes enhances the anti-infective activity of muramyldipeptides. 1985.

Escudero, J., and B. Hohn. 1997. Transfer and integration of T-DNA without cell injury in the host plant. Plant Cell. 9:2135-2142.

Fekete, D. M., and C. L. Cepko. 1993. Retroviral infection coupled with tissue transplantation limits gene transfer in the chick embryo. Proc. Natl. Acad. Sci. USA. 90:2350-2354.

Felgner, P. L., T. R. Gadek, M. Holm, R. Roman, et al. 1987. Lipofectin: A highly efficient, lipid-mediated DNA/transfection procedure. Proc. Natl. Acad. Sci. USA. 84:7413-7417.

Ferretti, P., and J. P. Brockes. 1988. Culture of newt cells from different tissues and their expression of a regeneration-associated antigen. The Journal of Experimental Zoology. 247:77-91.

Fieck, A., D. L. Wyborski, and J. M. Short. 1992. Modifications of the E. coli Lac repressor for expression in eukaryotic cells: effects of nuclear signal sequences on protein activity and nuclear accumulation. Nucleic Acids Res. 20:1785-91.

Finer, J. I., K. R. Finer, and T. Ponappa. 1999. Particle bombardment-mediated transformation. Current Topics in microbiology and immunzology. 240:59-80.

Fleer, R., P. Yeh, N. Amellal, I. Maury, et al. 1991. Stable multicopy vectors for high-level secretion of recombinant human serum albumin by Kluyveromyces yeasts. Biotechnology (N Y). 9:968-75.

U.S. Pat. No.5,804,604. Tat-derived transport polypeptides and fusion proteins. 1998.

Fromm, M., L. P. Taylor, and V. Walbot. 1985. Expression of genes transferred into monocot and dicot plant cells by electroporation. Proc. Natl. Acad. Sci. USA. 82:5824-5828.

Fujita, T., H. Shubiya, T. Ohashi, K. Yamanishi, et al. 1986. Regulation of human interleukin-2 gene: Functional DNA sequences in the 5′ flanking region for the gene expression in activated T lymphocytes. Cell. 46:401-407.

Gallagher, S. R. 1992. GUS protocols: Using the GUS gene as a reporter of gene expression. Academic Press, San Diego, Calif.

Gennaro, A. R. 2000. Remington: The science and practice of pharmacy. Lippincott, Williams & Wilkins, Philadelphia, Pa.

Gietz, R. D., R. A. Woods, P. Manivasakam, and R. H. Schiestl. 1998. Growth and transformation of Saccharoinyces cerevisiae. In Cells: A laboratory manual. Vol. I. D. Spector, R. Goldman, and L. Leinwand, editors. Cold Spring Harbor Press, Cold Spring Harbor, N.Y.

Gilbert, S. 1991. Developmental Biology. Sinauer Associates, Inc, Sunderland, Mass.

Goding, J. W. 1996. Monoclonal antibodies: Principles and Practice. Academic Press, San Diego. 492 pp.

Gorman, C. M., L. F. Moffat, and B. H. Howard. 1982. Recombinant genomes which express chloramphenicol acetyltransferase in mammalian cells. Mol. Cell. Biol. 2:1044-1051.

Goss, R., and M. Stagg. 1957. The regeneration of fins and fin rays in Fundulus heteroclitus. J Exp. Zool. 136:487-508.

Gossen, M., and H. Bujard. 1992. Tight control of gene expression in mammalian cells by tetracycline-responsive promoters. Proc Natl Acad Sci USA. 89:5547-5551.

Graham, F. L., and A. J. van der Eb. 1973. A new technique for the assay of infectivity of human adenovirus 5 DNA. Virology. 52:456-.

Griffin, K., R. Patient, and N. Holder. 1995. Analysis of FGF function in normal and no tail zebrafish embryos reveals separate mechanisms for formation of the trunk and the tail. Development. 121:2983-94.

Grigoriadis, A. E., J. N. Heersche, and J. E. Aubin. 1988. Differentiation of muscle, fat, cartilage, and bone from progenitor cells present in a bone-derived clonal cell population: effect of dexamethasone. J. Cell Biol. 106:2139-2151.

Guo, K., J. Wang, V. Andres, R. C. Smith, et al. 1995. MyoD-induced expression of p21 inhibits cyclin-dependent kinase activity upon myocyte terminal differentiation. Molecular and Cellular Biolog. 15:3823-3829.

Hanahan, D. 1983. Studies on transformation of Escherichia coli with plasmids. J. Mol. Biol. 166:557-580.

Hansen, G., and M. -D. Chilton. 1999. Lessons in gene transfer to plants by a gifted microbe. Curr. Top. Microbiol. Immunol. 240:21-57.

Hansen, G., and M. S. Wright. 1999. Recent advances in the transformation of plants. Trends Plant Sci. 4:226-231.

Harlow, E., and D. Lane. 1988. Antibodies: A laboratory manual. Cold Spring Harbor Laboratory Press, Cold Spring Harbor. 726 pp.

Harlow, E., and D. Lane. 1999. Using antibodies: A laboratory manual. Cold Spring Harbor Laboratory Press, Cold Spring Harbor, New York.

Hay, E. D., and D. A. Fischman. 1961. Origin of the blastema in regenerating limbs of the newt Triturus viridescens. An autoradiographic study using tritiated thymidine to follow cell proliferation and migration. Developmental Biology. 3:26-59.

Hill, R. E., P. F. Jones, A. R. Rees, C. M. Sime, et al. 1989. A new family of mouse homeobox-containing genes: molecular structure, chromosomal location, and developmental expression of Hox-7.1. Genes Dev. 3:26-37.

Hinnen, A., J. B. Hicks, and G. R. Fink. 1978. Transformation of yeast. Proc. Natl. Acad. Sci. USA. 75:1929-1933.

Hoffman, F. 1996. Laser microbeams for the manipulation of plant cells and subcellular structures. Plant Sci. 113:1-11.

Hoshimaru, M., J. Ray, D. W. Sah, and F. H. Gage. 1996. Differentiation of the immortalized adult neuronal progenitor cell line HC2S2 into neurons by regulatable suppression of the v-myc oncogene. Proc Natl Acad Sci U S A. 93:1518-1523.

Ishiura, M., S. Hirose, T. Uchida, Y. Hamada, et al. 1982. Phage particle-mediated gene transfer to cultured mammalian cells. Molecular and Cellular Biology. 2:601-616.

Ito, H., Y. Fukuda, K. Murata, and A. Kimura. 1983. Transformation of intact yeast cells treated with alkali cations. J. Bacteriol. 153:163-168.

Iujvidin, S., O. Fuchs, U. Nudel, and D. Yaffe. 1990. SV40 immortalizes myogenic cells: DNA synthesis and mitosis in differentiating myotubes. Differentiation 43:192-203.

Jaiswal, N., S. E. Haynesworth, A. I. Caplan, and S. P. Bruder. 1997. Osteogenic differentiation of purified, culture-expanded human mesenchymal stem cells in vitro. J Cell Biochem. 64:295-312.

Johnson, S. L., and J. A. Weston. 1995. Temperature-sensitive mutations that cause stage-specific defects in Zebrafish fin regeneration. Genetics. 141:1583-95.

Kaufman, R. J. 1990. Vectors used for expression in mammalian cells. Methods Enzymol. 185:487-511.

Kaufman, R. J., P. Murtha, D. E. Ingolia, C. -Y. Yeung, et al. 1986. Selection and amplification of heterologous genes encoding adenosine deaminase in mammalian cells. Proc. Natl. Acad. Sci. USA. 83:3136-3140.

Kawai, S., and M. Nishizawa. 1984. New procedure for DNA transfection with polycation and dimethyl sulfoxide. Mol. Cell. Biol. 4:1172.

Kelly, G., D. Erezyilmaz, and R. Moon. 1995. Induction of a secondary embryonic axis in zebrafish occurs following the overexpression of beta-catenin. Mech. Dev. 53:261-273.

Kelly, J. M., and M. J. Hynes. 1985. Transformation of Aspergillus niger by the amdS gene of Aspergillus nidulans. Embo J. 4:475-9.

Kettunen, P., and I. Thesleff. 1998. Expression and function of FGFs-4, -8, and -9 suggest functional redundancy and repetitive use as epithelial signals during tooth morphogenesis. Dev. Dyn. 211:256-268.

Koshiba, K., A. Kuroiwa, H. Yamamoto, K. Tamura, et al. 1998. Expression of Msx genes in regenerating and developing limbs of axolot1. J Exp Zool. 282:703-14.

Kozbor, D., P. Tripputi, J. C. Roder, and C. M. Croce. 1984. A human hybrid myeloma for production of human monoclonal antibodies. J Immunol. 133:3001-5.

Krauss, S., J. P. Concordet, and P. W. Ingham. 1993. A functionally conserved homolog of the Drosophila segment polarity gene hh is expressed in tissues with polarizing activity in zebrafish embryos. Cell. 75:1431-44.

Kriegler, M. 1990. Gene transfer and expression: A laboratory manual. Stockton Press, New York. 242 pp.

Kumar, A., C. P. Velloso, Y. Imokawa, and J. P. Brockes. 2000. Plasticity of retrovirus-labelled myotubes in the newt limb regeneration blastema. Developmental Biology. 218:125-136.

Laforest, L., C. W. Brown, G. Poleo, J. Geraudie, et al. 1998. Involvement of the sonic hedgehog, patched 1 and bmp2 genes in patterning of the zebrafish dermal fin rays. Development. 125:4175-84.

Leduc, N., and e. al. 1996. Isolated maize zygotes mimic in vivo embryogenic development and express microinjected genes when cultured in vitro. Dev. Biol. 10:190-203.

Lemischka, I. R., D. H. Raulet, and R. C. Mulligan. 1986. Developmental potential and dynamic behavior of hematopoietic stem cells. Cell. 45:917-927.

Liao, W., B. W. Bisgrove, H. Sawyer, B. Hug, et al. 1997. The zebrafish gene cloche acts upstream of a flk-1 homologue to regulate endothelial cell differentiation. Development. 124:381-9.

Littlefield, J. W. 1964. Selection of hybrids from matings of fibroblasts in vitro and their presumed recombinants. Science. 145:709-710.

Lo, D. C., F. Allen, and J. P. Brockes. 1993. Reversal of muscle differentiation during urodele limb regeneration. Proc Natl Acad Sci USA. 90:7230-7234.

Lopata, M. A., D. W. Cleveland, and B. Sollner-Webb. 1984. High-level expression of a chloramphenicol acetyltransferase gene by DEAEdextran-mediated DNA traansfection couled with a dimethylsulfoxide or glycerol shock treatment. Nucleic Acids Research. 12:5707.

Luckow, V. A. 1991. Cloning and expression of heterologous genes in insect cells with baculovirus vectors. In Recombinant DNA technology and applications. A. Prokop, R. K. Bajpai, and C. Ho, editors. McGraw-Hill, New York. 97-152.

Mackay, A. M., S. C. Beck, J. M. Murphy, F. P. Barry, et al. 1998. Chondrogenic differentiation of cultured human mesenchymal stem cells from marrow. Tissue Eng. 4:415-28.

Mandel, M., and A. Higa. 1970. Calcium-dependent bacteriophage DNA infection. J. Mol. biol. 53:159-162.

Martin, G. R. 1998. The roles of FGFs in the early development of vertebrate limbs. Genes Dev. 12:1571-86.

Miller, A. D., and C. Buttimore. 1986. Redesign of retrovirus packaging cell lines to avoid recombination leading to helper virus production. Mol. Cell biol. 6:2895-2902.

Miller, L. K. 1988. Baculoviruses as gene expression vectors. Annu. Rev. Microbiol. 42:177-199.

Milstein, C., and A. C. Cuello. 1983. Hybrid hybridomas and their use in immunohistochemistry. Nature. 305:537-40.

Mohammadi, M., G. McMahon, L. Sun, C. Tang, et al. 1997. Structures of the tyrosine kinase domain of fibroblast growth factor receptor in complex with inhibitors. Science. 276:955-60.

Moody, T. 1993. Growth factors, peptides, and receptors. Plenum Press, New York. 467 pp.

Morgan, T. H. 1901. Regeneration. Columbia University, New York. 316 pp.

Morrison, S. L., L. Wims, S. Wallick, L. Tan, et al. 1987. Genetically engineered antibody molecules and their application. Ann N Y Acad Sci. 507:187-98.

Munson, P. J., and D. Rodbard. 1980. Ligand: a versatile computerized approach for characterization of ligand-binding systems. Anal Biochem. 107:220-39.

U.S. Pat. No. 5,328,470. Treatment of diseases by site-specific instillation of cells or site-specific transformation of cells and kits therefor. 1994.

Neumann, E., M. Schaefer-Ridder, Y. Wang, and P. H. Hofschneider. 1982. Gene transfer into mouse lyoma cells by electroporation in high electric fields. EMBO J. 1:841-845.

Novitch, B. G., G. J. Mulligan, T. Jacks, and A. B. Lassar. 1996. Skeletal muscle cells lacking the retinoblastoma protein display defects in muscle gene expression and accumulate in S and G2 phases of the cell cycle. J Cell Biol. 135:441-456.

Ohuchi, H., T. Nakagawa, A. Yamamoto, A. Araga, et al. 1997. The mesenchymal factor, FGF10, initiates and maintains the outgrowth of the chick limb bud through interaction with FGF8, an apical ectodermal factor. Development. 124:2235-44.

O'Reilly, D. R., L. K. Miller, and V. A. Luckow. 1992. Baculovirus expression vectors. W.H. Freeman and Company, New York.

Ortega, S., M. Ittmann, S. H. Tsang, M. Ehrlich, et al. 1998. Neuronal defects and delayed wound healing in mice lacking fibroblast growth factor 2. Proc Natl Acad Sci U S A. 95:5672-7.

Ou-Lee, T. M., R. Turgeon, and R. Wu. 1986. Uptake and expression of a foreign gene linked to either a plant virus or Drosophila promoter in protoplasts of rice, wheat and sorghum. Proc. Natl. Acad. Sci. USA. 83:6815-6819.

Palmer, T. D., R. A. Hock, W. R. A. osborne, and A. D. Miller. 1987. Efficient retrovirus-mediated transfer and expression of a human adenosine deaminase gene in diploid skin fibroblasts from an adenosie-deficient human. Proc. Natl. Acad. Sci. USA. 84:1055-1059.

Pear, W., G. Nolan. M. Scott, and D. Baltimore. 1993. Production of high-titer helper-free retroviruses by transient transfection. Proc. Natl. Acad. Sci. USA. 90:8392-8396.

Peters, H., and R. Balling. 1999. Teeth. Where and how to make them. Trends Genet. 15:59-65.

Pittenger, M. F., A. M. Mackay, S. C. Beck, R. K. Jaiswal, et al. 1999. Multilineage potential of adult human mesenchymal stem cells. Science. 284:143-147.

Potter, H. 1988. Electroporation in biology: Methods, applications, and instrumentation. Analytical Biochemistry. 174:361-373.

Potter, H., L. Weir, and P. Leder. 1984. Enhancer-dependent expression of human kappa immunoglobulin genes introduced into mouse pre-B lymphocytes by electroporation. Proc. Natl. Acad. Sci. USA. 81:7161-7165.

Rassoulzadegan, M., B. Binetruy, and F. Cuzin. 1982. High frequency of gene transfer after fusion between bacteria and eukaryotic cells. Nature. 295:257.

Reifers, F., H. Bohli, E. C. Walsh, P. H. Crossley, et al. 1998. Fgf8 is mutated in zebrafish acerebellar (ace) mutants and is required for maintenance of midbrain-hindbrain boundary development and somitogenesis. Development. 125:2381-95.

Rhodes, C. A., D. A. Pierce, I. J. Mettler, D. Mascarenhas, et al. 1988. Genetically transformed maize plants from protoplasts. Science. 240:204-207.

Robert, B., D. Sassoon, B. Jacq, W. Gehring, et al. 1989. Hox-7, a mouse homeobox gene with a novel pattern of expression during embryogenesis. Embo J. 8:91-100.

Rose, J. K., L. Buonocore, and M. whitt. 1991. A new cationic liposome reagent mediating nearly quantitative transfection of animal cells. BioTechniques. 10:520-525.

Sambrook, J. 1989. Molecular cloning: a laboratory manual. Cold Spring Harbor Laboratory, Cold Spring Harbor.

Sandri-Goldin, R. M., A. L. Goldin, J. C. Glorioso, and M. Levine. 1981. High-frequency transfer of cloned herpes simplex virus type I sequences to mammalian cells by protoplast fusion. Mol. Cell. Biol. 1:7453-752.

Santamaria, J. A., and J. Becerra. 1991. Tail fin regeneration in teleosts: cell-extracellular matrix interaction in blastemal differentiation. J Anat. 176:9-21.

Saunders, J. A., B. F. Matthews, and P. D. Miller. 1989. Plant gene transfer using electrofusion and electroporation. In Electroporation and electrofusion in cell biology. E. Neumann, A. E. Sowers, and C. A. Jordan, editors. Plenum Press, New York. 343-354.

Schade, R., C. Staak, C. Hendriksen, M. Erhard, et al. 1996. The production of avian (egg yold) antibodies: IgY. The report and recommendations of ECVAM workshop. Alternatives to Laboratory Animals (ATLA). 24:925-934.

Schaffner, W. 1980. Direct transfer of cloned genes from bacteria to mammalian cells. Proc. Natl. Acad. Sci. USA. 77:2163.

Schneider, J. W., W. Gu, L. Zhu, V. Mahdavi, et al. 1994. Reversal of terminal differentiation mediated by p107 in Rb−/− muscle cells. Science. 264:1467-1471.

Schook, L. B. 1987. Monoclonal antibody production techniques and applications. Marcel Dekker, lnc., New York. 336 pp.

Selden, R. F., K. Burke-Howie, M. E. Rowe, H. M. Goodman, et al. 1986. Human growth hormone as a reporter gene in regulation studies employing transient gene expression. Molecular aid Cellular Biololgy. 6:3173-3179.

Shigekawa, K., and W. J. Dower. 1988. Electroporation of eukaryotes and prokaryotes: A general approach to the introduction of macomolecules into cells. BioTechniques. 6:742-751.

Shillito, R. 1999. Methods of genetic transformations: Electroporation and polyethylene glycol treatment. In Molecular improvement of cereal crop. I. Vasil, editor. Kluwer, Dordrecht, The Netherlands. 9-20.

Shilo, B. Z., and R. A. Weinberg. 1981. DNA sequences homologous to vertebrate oncogenes are conserved in Drosophila melanogaster. Proc Natl Acad Sci U S A. 78:6789-92.

Simon, H. G., C. Nelson, D. Goff, E. Laufer, et al. 1995. Differential expression of myogenic regulatory genes and Msx-1 during dedifferentiation and redifferentiation of regenerating amphibian limbs. Dev Dyn. 202:1-12.

Simonsen, C. C., and A. D. Levinson. 1983. Isolation and expression of an altered mouse dihydrofolate reductase cDNA. Proc. Natl. Acad. Sci. USA. 80:2495-2499.

Song, K., Y. Wang, and D. Sassoon. 1992. Expression of Hox-7.1 in myoblasts inhibits terminal differentiation and induces cell transformation. Nature. 360:477-481.

Southern, P. J., and P. Berg. 1982. Transformation of mammalian cells to antibiotic resistanced with a bacterial gene under control of the SV40 early region promoter. J. Mol. Appl Gen. 1:327-341.

Sreekrishna, K., R. H. Potenz, J. A. Cruze, W. R. McCombie, et al. 1988. High level expression of heterologous proteins in methylotrophic yeast Pichia pastoris. J Basic Microbiol. 28:265-78.

Stein, C. A., and J. S. Cohen. 1988. Oligodeoxynucleotides as inhibitors of gene expression: a review. Cancer Res. 48:2659-68.

Sun, L., N. Tran, C. Liang, F. Tang, et al. 1999. Design, synthesis, and evaluations of substituted 3-[(3- or 4- carboxyethylpyrrol-2-yl)methylidenyl]indolin-2-ones as inhibitors of VEGF, FGF, and PDGF receptor tyrosine kinases. J Med Chem. 42:5120-30.

Tanaka, E. M., A. A. Gann, P. B. Gates, and J. P. Brockes. 1997. Newt myotubes reenter the cell cycle by phosphorylation of the retinoblastoma protein. J Cell Biol. 136:155-65.

Thisse, B., C. Thisse, and J. A. Weston. 1995. Novel FGF receptor (Z-FGFR4) is dynamically expressed in mesoderm and neurectoderm during early zebrafish embryogenesis. Dev Dyn. 203:377-91.

Thompson, J. A., and e. al. 1995. Maize transformation utilizing silicon carbide whiskers: A review. Euphytica. 85:75-80.

Tiainen, M., D. Spitkovsky, P. Jansen-Durr, A. Sacchi, et al 1996. Expression of E1 A in terminally differentiated muscle cells reactivates the cell cycle and suppresses tissue-specific genes by separable mechanisms. Mol Cell Biol. 16:5302-5312.

Tilburn, J., C. Scazzocchio, G. G. Taylor, J. H. Zabicky-Zissman, et al. 1983. Transformation by integration in Aspergillus nidulans. Gene. 26:205-21.

Touraev, A., and e. al. 1997. Plant male germ line transformation. Plant J. 12:949-956.

Trick, H. N., and e. al. 1997. Recent advances in soybean transformation. Plant Tissue Cult. Biotechnol. 3:9-26.

Turner, D. L., E. Y. Snyder, and C. L. Cepko. 1990. Lineage-independent determination of cell type in the embryonic mouse retina. Neuron. 4:833-845.

van der Krol, A. R., J. N. Mol, and A. R. Stuitje. 1988. Modulation of eukaryotic gene expression by complementary RNA or DNA sequences. Biotechniques. 6:958-76.

Vogel, A., D. Roberts-Clarke, and L. Niswander. 1995. Effect of FGF on gene expression in chick limb bud cells in vivo and in vitro. Dev Biol. 171:507-20.

Vogel, A., C. Rodriguez, and J. C. Izpisua-Belmonte. 1996. Involvement of FGF-8 in initiation, outgrowth and patterning of the vertebrate limb. Development. 122:1737-50.

Walsh, K., and H. Perlman. 1997. Cell cycle exit upon myogenic differentiation. Curr Opin Genet Dev. 7:597-602.

Wang, Y., and D. Sassoon. 1995. Ectoderm-mesenchyme and mesenchyme-mesenchyme interactions regulate Msx-1 expression and cellular differentiation in the murine limb bud. Dev Biol. 168:374-82.

Wells, J. A., M. Vasser, and D. B. Powers. 1985. Cassette mutagenesis: an efficient method for generation of multiple mutations at defined sites. Gene. 34:315-23.

Whitt, M. A., L. Buonocore, J. K. Rose, V. Ciccarone, et al. 1990. TransfectACE reagent promotes transient transfection frequencies greater than 90%. Focus. 13:8-12.

Wigler, M., A. Pellicer, S. Silversttein, and R. Axel. 1978. Biochemical transfer of single-copy eucaryotic genes using total cellular DNA as donor. Cell. 14:725.

Williams, D. A., I. R. Lemischka, D. G. Nathan, and R. C. Mulligan. 1984. Introduction of a new genetic material into pluripotent haematopoietic stem cells of the mouse. Nature. 310:476-480.

Woloshin, P., K. Song, C. Degnin, A. M. Killary, et al. 1995. MSX1 inhibits myoD expression in fibroblast x 10T1/2 cell hybrids. Cell. 82:611-620.

Wong, T. K., and E. Neumann. 1982. Electric field mediated gene transfer. Biochemical and Biophysical Research Communications. 107:584-587.

Wyborski, D. L., L. C. DuCocur, and J. M. Short. 1996. Parameters affecting the use of the lac repressor system in eukaryotic cells and transgenic animals. Environ Mol Mutagen. 28:447-58.

Wyborski, D. L., and J. M. Short. 1991. Analysis of inducers of the E.coli lac repressor system in mammalian cells and whole animals. Nucleic Acids Res. 19:4647-53.

Yelton, M. M., J. E. Haamer, and W. E. Timberlake. 1984. Transformation of Aspergillus nidulans by using a trpC plasmid. Proc Natl Acad Sci U S A. 81:1470-4.

Zetter, B. R. 1998. Angiogenesis and tumor metastasis. Annu Rev Med. 49:407-24.

Zhou, G., and e. al. 1983. Introduction of exogenous DNA into cotton embryos. Methods Enzymol. 101:433-481.

Zhu, X., J. Sasse, D. McAllister, and J. Lough. 1996. Evidence that fibroblast growth factors 1 and 4 participate in regulation of cardiogenesis. Dev Dyn. 207:429-38.

Zoller, M. J., and M. Smith. 1987. Oligonucleotide-directed mutagenesis: a simple method using two oligonucleotide primers and a single-stranded DNA template. Methods Enzymol. 154:329-50.

Zottoli, S. J., A. P. Bentley, D. G. Feiner, J. R. Hering, et al 1994. Spinal cord regeneration in adult goldfish: implications for functional recovery in vertebrates. Prog Brain Res. 103:219-28.

1. A method of regenerating mammalian tissue, comprising
dedifferentiating differentiated mammalian cells by contacting them with a composition capable of inducing dedifferentiation, regeneration or both,
wherein following dedifferentiation the mammalian cells are capable of proliferating and regenerating into redifferentiated or newly differentiated mammalian cells.

2. The method of claim 1, further comprising subsequently proliferating the dedifferentiated mammalian cells.

3. The method of claim 2, further comprising regenerating mammalian cells, tissue, or organs from the dedifferentiated mammalian cells.

4. The method of claim 1, wherein dedifferentiating is conducted in vivo

5. The method of claim 1, wherein dedifferentiating is conducted ex vivo.

6. The method of claim 1, wherein dedifferentiating comprises contacting the mammalian cells with the composition capable of inducing dedifferentiation for a time sufficient to induce dedifferentiation.

7. The method of claim 1, wherein the dedifferentiating is conducted at the site of an injury.

8. The method of claim 7, wherein the injury is caused by disease or trauma.

9. The method of claim 1, wherein the contacting comprises injecting the composition into the site of injury.

10. The method of claim 1, wherein the contacting comprises injecting the composition systemically.

11. The method of claim 1, wherein the contacting comprises topically applying the composition to the site of injury.

12. The method of claim 1, wherein the contacting comprises implanting a delivery device.

13. The method of claim 1, wherein the mammalian cells are isolated from muscle, skin, bone, joints, eye, lung, heart, vasculature, kidney, pancreas, or nervous tissue.

14. The method of claim 1, wherein the mammalian cells are muscle cells.

15. The method of claim 1, wherein the composition comprises an active polypeptide which is a fibroblast growth factor, a fibroblast growth factor receptor, a bone morphogenic polyp eptide, a bone morphogenic polyp eptide receptor, a Wnt polypeptide, a metalloproteinase polypeptide, msx1, msx2, E2F, frizzled, a SMAD polypeptide or a fatty acid binding polypeptide.

16. The method of claim 15, wherein the active polypeptide is a fusion polypeptide.

17. The method of claim 16, wherein the fusion polypeptide comprises the active polypeptide and a polypeptide that facilitates introduction into said cells.

18. The method of claim 1, wherein the composition comprises a polynucleotide encoding an active polypeptide, wherein the active polypeptide is a fibroblast growth factor, a fibroblast growth factor receptor, a bone morphogenic polypeptide, a bone morphogenic polypeptide receptor, a Wnt polypeptide, a metalloproteinase polypeptide, msx1, msx2, E2F, frizzled, a SMAD polypeptide or a fatty acid binding polypeptide.

19. The method of claim 18, wherein the polynucleotide is operably linked to a promoter.

20. The method of claim 19, wherein the promoter is an inducible promoter.

21. The method of claim 18, wherein the polynucleotide is in a vector.

22. The method of claim 15 or 18, wherein the active polypeptide is msx-1.

23. The method of claim 15 or 18, wherein the active polypeptide is fibroblast growth factor.

24. The method of claim 15 or 18, comprising 2 or more active polypeptides.

25. The method of claim 15 or 18, comprising 3 or more active polypeptides.

26. A composition comprising a carrier and a polypeptide which is a fibroblast growth factor, a fibroblast growth factor receptor, a bone morphogenic polypeptide, a bone morphogenic polypeptide receptor, a Wnt polypeptide, a metalloproteinase polypeptide, msx1, msx2, E2F, frizzled, a SMAD polypeptide or a fatty acid binding polypeptide,
wherein the composition dedifferentiates a mammalian cell.

27. A method, comprising
dedifferentiating differentiated mammalian cells by contacting them with a composition comprising an extract from the regeneration site of an animal such that the composition or extract induces dedifferentiation, regeneration or both,
wherein following dedifferentiation the mammalian cells can proliferate and regenerate into redifferentiated mammalian cells.

28. The method of claim 27, further comprising subsequently proliferating the dedifferentiated mammalian cells.

29. The method of claim 28, further comprising regenerating mammalian cells, a tissue or an organ from the dedifferentiated cells.

30. The method of claim 27, wherein dedifferentiating is conducted in vivo

31. The method of claim 27, wherein dedifferentiating is conducted ex vivo.

32. A composition comprising a carrier and an extract from a regenerating site of an animal, wherein the extract dedifferentiates differentiated mammalian cells.

33. A method of identifying polypeptides that induce dedifferentiation of mammalian cells, comprising:
extracting cells from the regeneration site of an animal,
purifying components of the extract,
applying the purified components to mammalian cells,
observing the amount, if any, of dedifferentiation of the mammalian cells, and
comparing the obtained amount of dedifferentiation to the amount of dedifferentiation achieved by contacting mammalian cells with an extract from a newt regenerating site, wherein about the same or greater dedifferentiating activity indicates the polypeptide is capable of inducing dedifferentiation, regeneration or both.

34. A patch comprising,
a matrix, and
an extract from regenerating site of an animal, wherein the extract dedifferentiates differentiated mammalian cells.

35. The invention of claim 1, 26, 27, 32, 33 or 34, wherein the extract is an extract from urodeles, teleost fish, echinoderms, and crustaceans.

36. The method of claim 35, wherein the extract is an extract from a newt.

37. The method of claim 35, wherein the extract is humanized.

38. A method, comprising dedifferentiating differentiated myotube cells by contacting them with a composition comprising an extract from a regeneration site of newt limbs such that the composition induces dedifferentiation, regeneration or both.

39. The method of 38, wherein said myotube cells are murine.

40. The method of 39, wherein said myotube cells are C2C12 cells.

41. The method of 38, wherein said myotube cells are newt.

42. The method of 38, where in said cells are cultured in vitro.

43. The method of 38, wherein after said dedifferentiation, the myotube cells proliferate.

44. A method, comprising dedifferentiating differentiated myotube cells by contacting said cells with a composition comprising a msx1 polynucleotide.

45. The method of 44, wherein said msx1 polynucleotide is operably-linked to an inducible promoter.

46. The method of 44, wherein said myotube cells are murine.

47. The method of 44, wherein said myotube cells are cultured in vitro.

48. The method of 44, wherein after said dedifferentiation, the myotube cells proliferate.

49. The method of 44, wherein after said dedifferentiation, said cells are pluripotent.

50. A method, comprising inducing blastema formation at an injury site by contacting the injury site with a composition comprising fibroblast growth factor.

51. The method of 50, wherein said fibroblast growth factor is wound fibroblast growth factor.

52. A method comprising inhibiting blastema formation at a site of injury by contacting said site with an inhibitor of fibroblast growth factor receptors.

53. The method of 52, wherein said inhibitor is SU5402.

54. The method of 50 or 52, wherein said injury is in zebrafish.

55. The method of 50 or 52, wherein said injury is incurred by trauma or disease.
